(19) United States
(12) Patent Application Publication
(10) Pub. No.: US 2025/0228899 A1
(43) Pub. Date: Jul. 17, 2025

(54) GENERATING AND UTILIZING ADULT HEPATOCYTE ORGANOIDS

(71) Applicant: University of Washington, Seattle, WA (US)

(72) Inventors: Kelly Rose Stevens, Seattle, WA (US); Sarah Henley Saxton, Seattle, WA (US); Shannon Nicole Mitchell, Seattle, WA (US); Inkyung Kang, Seattle, WA (US)

(73) Assignee: University of Washington, Seattle, WA (US)

(21) Appl. No.: 18/703,023

(22) PCT Filed: Oct. 20, 2022
(86) PCT No.: PCT/US2022/078469

§ 371 Date: Apr. 19, 2024

Related U.S. Application Data
(60) Provisional application No. 63/270,431, filed on Oct. 21, 2021.

(51) Int. Cl. A61K 35/407; C12N 5/071

(52) U.S. Cl. CPC A61K 35/407; C12N 5/0671; C12N 2501/11; C12N 2501/415; C12N 2533/56; C12N 2533/90

(57) ABSTRACT

Techniques for generating and utilizing adult hepatocyte organoids are described. An example method includes generating an organoid by culturing hepatocytes obtained from an adult donor in a hepatocyte culture medium. The hepatocyte culture medium includes a basal medium and 5 μM of a TGF-β inhibitor.

### CROSS-REFERENCE TO RELATED APPLICATION(S)

This application is a U.S. National Phase Patent Application based on International Patent Application No. PCT/US2022/078469, which claims the priority of U.S. Provisional App. No. 63/270,431, titled “METHODS TO CULTURE AND GRAFT ADULT HEPATOCYTE ORGANOIDS” and filed on Oct. 21, 2021, each of which is incorporated by reference herein in its entirety.

### REFERENCE TO SEQUENCE LISTING

The Sequence Listing associated with this application is provided in XML format in lieu of a paper copy and is hereby incorporated by reference into the specification. The name of the XML file containing the Sequence Listing is 31I3894.xml. The XML file is 16,384 bytes, was created on Apr. 11, 2024, and is being submitted electronically via Patent Center.

### TECHNICAL FIELD

This application relates to techniques for generating and utilizing organoids grown from adult hepatocytes.

### BACKGROUND

As the largest solid organ in the body with hundreds of wide-ranging functions, the liver is essential for the maintenance of healthy human life (Arias, I. M. et al. (2020). The Liver: Biology and Pathobiology (John Wiley & Sons)). Some of the liver's roles include synthesis of plasma proteins, blood detoxification, drug metabolism, storage of glycogen, bile production and excretion, and cholesterol synthesis. Due to the high functional and metabolic demands on the liver, hepatic injury can have systemic and devastating effects on human health. Liver disease currently causes 3.5% of global mortality, with incidence of both chronic and acute disease continuing to increase annually (Asrani, S. K. et al. (2019). Journal of Hepatology 70, 151-171). Those living with liver disease experience a severely reduced quality of life, which results in a significant and inequitable healthcare burden across the US and the world (Asrani et al., 2019; Dakhoul, L. et al. (2019). Hepatology Communications 3, 52-62; Nephew, L. D. and Serper, M. (2021). Liver Transplantation 27, 900-912; Stepanova, M. et al. (2017). Clinical Gastroenterology and Hepatology 15, 759-766.e5).

The vast majority of the liver's critical metabolic functions are performed by hepatocytes, an epithelial cell comprising ˜80% of the liver parenchyma. The functional tasks carried out by hepatocytes arise progressively over human development, in tandem with human hepatocyte fate specification and maturation, with mature adult hepatocytes ultimately performing ˜500 functions (Arias et al., 2020). Due to their hundreds of critical roles in human health, a source of mature human hepatocytes for research and translational applications would enable countless studies, ranging from basic liver biology to drug development to therapeutics such as implantable cell-based therapies for treating liver disease (Dwyer, B. J. et al. (2021). Journal of Hepatology 74, 185-199).

To address this need, numerous human hepatocyte sources have been explored. Yet, several of these, such as induced pluripotent stem cell (iPSC)-derived hepatocytes and fetal hepatocytes, remain immature in phenotype and function. The immaturity of these cells makes them unsuitable for many applications requiring functionality or robust engraftment in vivo, as maturity highly correlates with engraftment success (Baxter, M. et al. (2015). Journal of Hepatology 62, 581-589; Billerbeck, E. et al. (2016). Journal of Hepatology 65, 334-343; Haridass, D. et al. (2009). The American Journal of Pathology 175, 1483-1492). Conversely, primary human hepatocytes isolated from adult livers are functionally mature (Billerbeck et al., 2016; Dwyer et al., 2021; Haridass et al., 2009; Iansante, V. et al. (2018). Biological Sciences 373), but cannot currently be propagated extensively in vitro. Moreover, these cells are typically structurally and functionally impaired upon recovery from cryopreservation and lose their morphology and functionality over days in culture unless supported by highly specialized culture methods or equipment (Khetani, S. R. and Bhatia, S. N. (2008). Nature Biotechnology 26, 120-126; Stéphenne, X. et al. (2010). World Journal of Gastroenterology: WJG 16, 1; Terry et al., 2007; Underhill, G. H. and Khetani, S. R. (2018). Cellular and Molecular Gastroenterology and Hepatology 5, 426-439.e1). A reliable method to culture mature human hepatocytes in vitro in a manner that sustains their functionality and potential for later engraftment in vivo could transform basic liver biology and regenerative medicine.

One promising approach could be to leverage organoids—3D “mini-organs” that have revolutionized culture of numerous primary epithelial cell types, ranging from the human intestine to kidney (Hofer, M. and Lutolf, M. P. (2021). Nature Reviews Materials 2021 6:5 6, 402-420; Homan, K. A. et al. (2019). Nature Methods 1; Huch, M. et al. (2015). Cell 160, 299-312; Marsee, A. et al. (2021). Cell Stem Cell 28, 816-832; Sato, T. et al. (2011). Gastroenterology 141, 1762-1772). In the liver field, human organoid culture methods have been developed that recapitulate several stages of embryonic and fetal hepatic development, including hepatoblasts (progenitor cells in the embryonic and fetal liver, gestational weeks 3-7) (Wesley, B. T. et al. (2022) BioRxiv 2022.03.08.482299), fetal hepatocytes (gestational weeks 7-20), and one pediatric donor (Hu, H. et al. (2018). Cell 175). In adult settings, liver organoids have been generated from cholangiocytes, which are the epithelial cells lining the liver's bile ducts that share epithelial signatures with hepatocytes but are otherwise functionally and phenotypically distinct (Arias et al., 2020; Huch et al., 2015; Sampaziotis, F. et al. (2017). Nature Medicine 23, 954). Thus far, reproducibly generating organoid cultures from adult human hepatocytes that replicate the diverse and numerous functions of mature human liver has remained elusive.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 illustrates an example environment for generating and utilizing organoids.

FIG. 2 illustrates an example system for generating organoids.

FIG. 3 illustrates an example process for generating an organoid using adult hepatocytes.

FIG. 4 illustrates an example process for implanting an adult hepatocyte organoid in a subject.

FIG. 5 illustrates an example process for evaluating a therapeutic agent using an adult hepatocyte organoid.

FIGS. 6A to 6F illustrate that adult human hepatocytes exhibited significant growth and maintenance of phenotype and morphology through organoid culture.

FIGS. 7A to 7I illustrate optimized adult human hepatocyte organoid culture conditions identified using in vitro screening, according to some example implementations.

FIGS. 8A to 8I illustrate adult human hepatocyte organoid growth tracking and phenotypic characterization.

FIGS. 9A and 9B show that human hepatocytes demonstrated variability in 2D plating and albumin secretion.

FIGS. 10A to 10H show that adult human hepatocyte organoids from seven separate human donors formed reliably and demonstrated robust hepatic functions.

FIGS. 11A to 11G illustrate results of single-cell RNA sequencing demonstrating transcriptomic maturity and functionality of adult human hepatocytes grown as organoids in accordance with an example.

FIGS. 12A to 12F illustrate that cell RNA sequencing demonstrates cellular composition in adult human hepatocyte organoids and published human liver samples.

FIGS. 13A to 13G show that engineered liver tissues generated with adult human hepatocyte organoids demonstrate increased function and engraftment than those generated with hepatocyte aggregates.

FIGS. 14A to 14C show the results of engineered liver tissues fabricated with adult human hepatocyte organoids engraft and function in vivo in a mouse model of chronic liver injury.

FIGS. 15A and 15B shows the results of adult human hepatocyte organoids and aggregates with and without non-parenchymal cells before implantation in FRGN mice.

FIG. 16 illustrates albumin secretion of hepatocytes grown in 11 different culture medias measured by human albumin ELISA.

FIG. 17 illustrates average cross-sectional area of organoids in individual wells grown in in 11 different culture medias.

FIG. 18 illustrates an example of albumin secretion measured by human Albumin ELISA, comparing adult human hepatocyte organoids grown in 6 different concentrations of A83-01 ranging from 0 nM to 40 nM.

FIG. 19 illustrates an example of average organoid size of adult human hepatocyte organoids grown in four different concentrations of A83-01, ranging from 0 nM to 100 nM.

FIG. 20 illustrates an example of albumin secretion measured by human albumin ELISA comparing adult human hepatocyte organoids grown in eight different medium combinations with eight different concentrations of A83-01, ranging from 0 nM to 50 nM.

FIG. 21 illustrates an example of albumin secretion measured by human albumin ELISA comparing adult human hepatocyte organoids grown in media with four different concentrations of TNF-α.

FIG. 22 illustrates an example of albumin secretion measured by human albumin ELISA comparing adult human hepatocyte organoids grown in media with different growth factor compositions, including Hepatocyte Growth Factor (HGF) and Fibroblast Growth Factor 10 (FGF10).

FIG. 23 illustrates an example of average cross-sectional area of organoids in individual wells from day 1 to day 14 after exposure to P. vivax malaria parasites.

FIG. 24 illustrates an example of albumin secretion measured by human albumin ELISA from day 2 to day 14 after exposure to P. vivax malaria parasites.

FIG. 25 illustrates an example of the recovery of human adult hepatocyte organoids after cryopreservation.

### DETAILED DESCRIPTION

Novel adult human hepatocyte organoid culture methods are described herein. These methods generate adult human hepatocyte organoids that closely maintain the phenotype, morphology, transcription, and functional characteristics of hepatocytes in the human liver. The adult human hepatocyte organoids maintain numerous characteristics of mature human liver function including protein production, nitrogen metabolism, drug metabolism, cholesterol synthesis, and glycogen storage.

Various implementations of the present disclosure further relate to media formulations configured to culture adult hepatocytes into organoids. Particular media formulations described herein are configured to sustain adult hepatocyte cultures in vitro. The engineered liver tissues containing adult human hepatocyte organoids described herein generate substantively larger and more functional hepatic grafts after implantation compared to those created using prior methods (see, e.g., Stevens, K. R. et al. (2017). Sci Transl Med 9, eaah5505). Various implementations of the present disclosure further relate to liver tissue grafts including adult human hepatocyte organoids.

Various implementations of the present disclosure provide numerous improvements over existing techniques. Previously reported culture medium and techniques, such as those described in PCT/EP2019/071574 (titled “BILIARY ORGANOIDS”), are unable to sustain mature human hepatocytes obtained from adult donors. In contrast, culture medium formulations and other techniques described herein are experimentally verified to grow adult human hepatocytes in vitro. Thus, implementations of the present disclosure enable the growth of organoids from differentiated hepatocytes.

According to some implementations described herein, the organoids described herein are grown and/or maintained in conditions that cause the organoids to act similarly to adult liver tissue. As a result, the organoids provide new mechanisms for producing organ tissue for liver transplants, for liver tissue grafts, for toxicity studies, for drug development, for infectious disease studies, and other research applications.

The organoids described herein are different to previously reported tissues in several respects. In particular implementations, the adult human hepatocyte organoids described herein have similar functionality and metabolism to in vivo liver tissue. For instance, this disclosure confirms the maturity of adult human hepatocyte organoids generated using techniques described herein by presenting experimental verification (obtained using gene expression analysis and direct measurement of functional activity) of the maintenance of drug-metabolizing enzymes.

In various implementations, grafts generated using these organoids provide different results than grafts prepared using cells prepared using other techniques. Thus, organoids described herein have powerful translational implications, as enhancing hepatic engraftment efficiency would concomitantly reduce the cell sourcing production burden for various research and therapeutic applications.

Implementations of the present disclosure will now be described with reference to the accompanying figures.

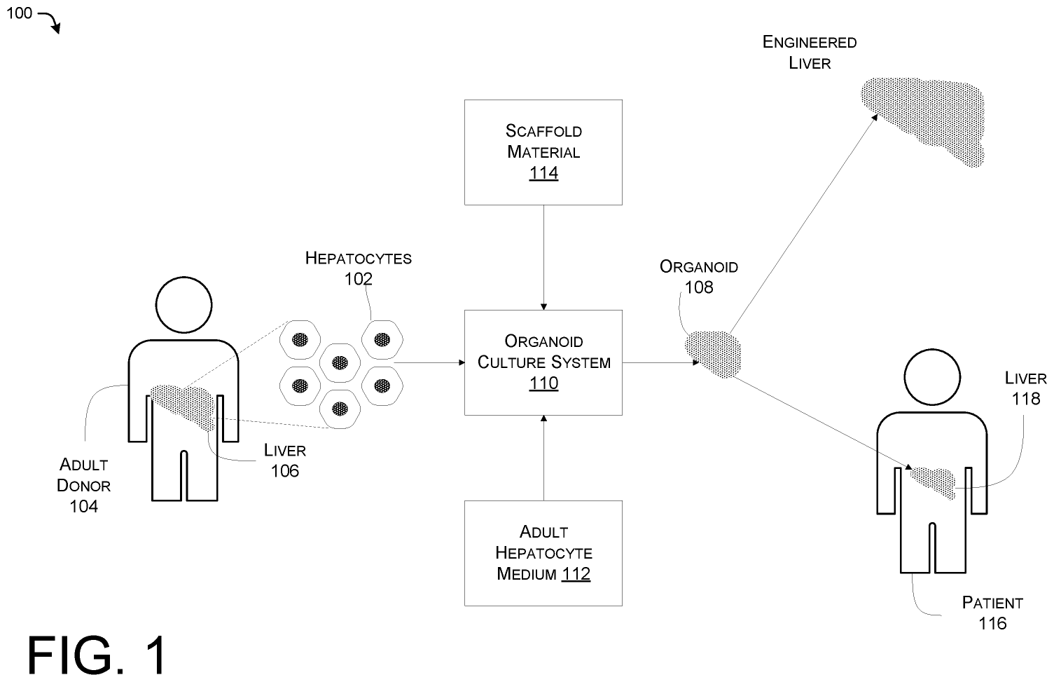

FIG. 1 illustrates an example environment 100 for generating and utilizing organoids. In various implementations, hepatocytes 102 are obtained from an adult donor 104. As used herein, the term “hepatocytes” refers to differentiated liver cells that perform various metabolic functions of the liver. The hepatocytes 102, for instance, are at least partially obtained from a liver 106 of the adult donor 104. In some implementations, the adult donor 104 is a human, but implementations are not so limited. For instance, the hepatocytes 102 are obtained by a biopsy of the liver 106 of the adult donor 104. In some implementations, a single hepatocyte 102 is obtained from the adult donor 104. For instance, the number of hepatocytes 102 is in a range of 1 to 1 billion. In various implementations, the hepatocytes 102 are isolated from the liver 106.

In some implementations, the hepatocytes 102 are not part of a liver tissue suitable for transplant. For instance, the hepatocytes 102 include and/or are a portion of a tissue that is of an insufficient quality and/or size for direct liver transplant. Accordingly, implementations of the present disclosure can utilize tissues that would otherwise have limited therapeutic utility.

According to various implementations of the present disclosure, the hepatocytes 102 are distinct from biliary progenitor cells, stem cells, and hepatoblasts. Biliary progenitor cells are a type of stem cell that are capable of differentiating into hepatocytes and cholangiocytes. In contrast, the hepatocytes 102 are already differentiated. Furthermore, the hepatocytes 102 are not obtained from fetal sources, like hepatoblasts. The hepatocytes 102 have differentiated into a mature phenotype by the time they are obtained from the adult donor(s) 104.

In some implementations, the hepatocytes 102 are temporarily cryopreserved. As used herein, the terms “cryopreserved, “cryoconserved,” and their equivalents, refer to the state of a biological tissue that has been preserved by cooling and maintaining the tissue at a temperature that is low enough to halt or significantly reduce biological and/or chemical reactions within the tissue. For example, a cryopreserved tissue is cooled and/or maintained at a temperature that is below −20° C. In various cases, the hepatocytes 102 are at a temperature that is in a range of −100° C. to −20° C. In some cases, the hepatocytes 102 are thawed after cryopreservation. For instance, the hepatocytes 102 are thawed rapidly and diluted out of dimethyl sulfoxide (DMSO).

According to various implementations of the present disclosure, an organoid 108 is generated using the hepatocytes 102. As used herein, the term “organoid,” and its equivalents, may refer to an in vitro a collection of differentiated cells that are self-organized into a 3D structure consistent with an organ. The organoid 108, for example, is functionally and structurally consistent with a liver. A mature liver includes epithelial cells (e.g., hepatocytes and cholangiocytes) that function together with endothelial, mesenchymal, and stromal cells to perform various metabolic, endocrine, and exocrine functions. Hepatocytes are arranged in densely packed chord-like structures in close proximity to supportive cells in the liver (e.g., cholangiocytes and vascular cells). Cholangiocytes are arranged into small ducts connected to larger ducts, which are in turn connected to the common hepatic duct. Bile generated by the cells exits the liver via the common bile duct. Blood is supplied to the cells via the vasculature via the hepatic artery and hepatic portal vein. Mature liver is highly vascularized. In various implementations, the organoid 108 includes hepatocytes arranged into self-assembled clumps that are consistent with at least portions of a mature liver. The organoid 108 may also include other liver cell populations such as cholangiocytes, which can be scattered throughout the organoid or arranged in small ducts or tubes, as well as other cells such as vascular cells or stromal cells.

Moreover, the organoid 108 may have several metabolic characteristics that are consistent with the mature liver. For example the organoid 108 may synthesize plasma proteins, detoxify blood when it flows through the organoid 108, metabolize drugs exposed to the organoid 108, store glycogen, produce and excrete bile, synthesize cholesterol, or any combination thereof.

In various implementations, the organoid 108 has relatively low expression of fetal genes, such as AFP, H19, and GPC3. These genes are also upregulated when hepatic cells become cancerous. Accordingly, the cells within the organoid 108 are adult cells without adopting a malignant transcriptional profile.

To generate the organoid 108, the hepatocytes 102 are transferred to an organoid culture system 110. In various cases, the hepatocytes 102 are gently transferred to the organoid culture system 110.

The organoid culture system 110, for example, includes a bioreactor, one or more well plates, flasks, or other mechanisms for culturing the hepatocytes 102. In some implementations, the organoid culture system 110 is actively manipulated and/or monitored by a user, such as a lab technician. In some cases, the organoid culture system 110 is at least partially automated.

In various implementations, the hepatocytes 102 are cultured in adult hepatocyte medium 112. As used herein, the terms “culture medium,” “medium,” and “media” are used interchangeably to refer to an aqueous solution that supports cell growth. The adult hepatocyte medium 112 may be a solution with multiple constituent solutes. According to some examples, the adult hepatocyte medium 112 includes nutrients that are metabolized by the hepatocytes 102 and/or enhance metabolism of the hepatocytes 102. For instance, the adult hepatocyte medium 112 includes carbohydrates, sugars, proteins, polypeptides, amino acids, vitamins, sodium pyruvate, or any combination thereof. In some cases, the adult hepatocyte medium 112 includes N-acetyl cystine and/or glucose. In particular implementations, the adult hepatocyte medium 112 includes a basal medium, such as at least one of Dulbecco's Modified Eagle Medium/Nutrient Mixture (DMEM) (from ThermoFisher Scientific of Waltham, MA), William's E media (from ThermoFisher Scientific of Waltham, MA), or F-12. The basal medium, in some cases, includes or one or more buffers (e.g., 2-[4-(2-Hydroxyethyl)piperazin-1-yl]ethane-1-sulfonic acid (HEPES) at a concentration of 1×). The basal medium may further include one or more additives. In various implementations, the adult hepatocyte medium 112 includes one or more antibiotics. For instance, the adult hepatocyte medium 112 includes penicillin, streptomycin, or a combination thereof. For instance, the basal medium includes 1× penicillin-streptomycin. In various implementations, the adult hepatocyte medium 112 includes one or more cell culture supplements, such as GlutaMAX® (from ThermoFisher Scientific of Waltham, MA), B27™ (from ThermoFisher Scientific of Waltham, MA), nicotinamide, N-acetyl cysteine, gastrin, or any combination thereof. In some cases, the basal medium includes 1× GlutaMAX®, 1-5% (by volume) B27, 5-10 millimoles (mM) nicotinamide, 1-1.5 mM N-acetyl cysteine, 5-15 nanomoles (nM) gastrin. In some cases, the basal media includes additional glucose. The basal medium, for instance, may be 30-50% of the adult hepatocyte medium 112.

In various cases, the adult hepatocyte medium 112 includes one or more growth factors, such as an epidermal growth factor (EGF), hepatocyte growth factor (HGF), transforming growth factor α (TGF-α), insulin growth factor (IGF), and/or at least one fibroblast growth factor (FGF) (e.g., FGF1, FGF2, FGF7, or FGF10). For instance, the adult hepatocyte medium 112 includes 25-75 nanograms per milliliter (ng/mL) EGF. In some cases, the adult hepatocyte medium 112 includes 1-100 nM of FGF. In various implementations, the adult hepatocyte medium 112 includes 1-100 nM tumor necrosis factor α (TNF-α). In various implementations, the adult hepatocyte medium includes other potentiators or inhibitors, some associated with liver regeneration, such as Interleukin-6 (IL-6), Vascular endothelial growth factor (VEGF), Insulin-like growth factors and insulin (e.g., IGF-I, IGF-II, insulin), PGE-2, IL1-β, Notch ligands, and, Angiopoietin-2 (Ang-2), and Activins. In various implementations, the adult hepatocyte medium 112 includes at least one TGF-β inhibitor. In some cases, the TGF-β inhibitor includes A83-01. For example, the TGF-β inhibitor is included at a concentration of 0.1 to 50.0 μM, 0.2 to 20.0 μM, 0.3 to 10.0 μM or 1.0 to 6.0 μM. In particular cases, the TGF-β inhibitor is included at a concentration of 5 μM. In various implementations, the adult hepatocyte medium includes other proteins, small molecules, or biological molecules that stimulate or inhibit the EGF, HGF, FGF, TNF-α, or TGF-β pathways.

In various cases, the adult hepatocyte medium 112 includes one or more non-canonical Wnt and/or β-Catenin pathway signaling potentiator and/or a canonical Wnt and/or β-Catenin pathway potentiator. For instance, the adult hepatocyte medium 112 includes Rspondin1 recombinant protein and/or Wnt3a recombinant protein. In some cases, 40-60% of the adult hepatocyte medium 112 is a canonical Wnt potentiator-conditioned medium and 5-15% of the adult hepatocyte medium 112 is a non-canonical Wnt potentiator-conditioned medium. In some cases, the media includes small molecules or other biological molecules that potential Wnt signaling pathways, including but not limited to CHIR99021.

The adult hepatocyte medium 112, in various implementations, includes one or more rho-associated protein kinase (ROCK) inhibitors. For instance, the adult hepatocyte medium 112 includes 5-10 μM of y-27632.

Additional solutes or biological molecules can be included in the adult hepatocyte medium 112. In some implementations, the adult hepatocyte medium 112 includes an interleukin (IL) (e.g., IL6), oncostatin-M (OSM), insulin, norepinephrine, glucagon, dexamethasone, bile acids or bile salts, or any combination thereof. In various implementations, the concentration of any of the components of the adult hepatocyte medium 112 can change over time as the hepatocytes 102 are growing into the organoid 108 within the organoid culture system 110.

In particular cases, the hepatocytes 102 are further cultured in the presence of a scaffold material 114. For instance, the scaffold material 114 includes natural and/or biological extracellular matrix (e.g., a solubilized basement membrane extracted from Engelbreth-Holm-Swarm (EHS) mouse sarcoma, such as MATRIGEL® (from Corning Inc. of Corning, NY) and/or GELTREX® (from Thermo Fisher Scientific Inc. of Waltham, MA)), collagen, fibrin, vitronectin, a gelatin-based material (e.g., gelatin methacryloyl (GelMA)), a polyethylene glycol (PEG)-based material (e.g., PEGDA), a synthetic or extracellular matrix (e.g., polyethylene glycol-based or gelatin methacrylate-based hydrogels), or any combination thereof. In some cases, the cell culture media or organoid culture systems contain factors that potentiate or inhibit extracellular matrix signaling or cellular adhesion signaling, such as antibodies, peptides, or small molecules that ligate integrins or cellular adhesion motifs. The scaffold material 114, for example, is configured to provide a similar mechanical environment to liver tissue within the organoid culture system 110. In various implementations, the hepatocytes 102 are cultured in 40-50% adult hepatocyte medium 112 and 50-60% scaffold material 114.

In some cases, the hepatocytes 102 are cultured in the presence of one or more additional cell types. The additional cell type(s) may include supportive cell types. For instance, the organoid 108 may be generated by culturing the hepatocytes 102 in the presence of cholangiocytes, fibroblasts, endothelial cells, macrophages, natural killer (NK) cells, stellate cells, Kupffer cells, neurons, or adipocytes. In some cases, the cholangiocytes, fibroblasts, endothelial cells, macrophages, NK cells, stellate cells, Kupffer cells, neurons, or adipocytes are also obtained from the adult donor 106. Accordingly, the organoid 108 may include the additional cell type(s).

The organoid 108, in various cases, is transferred out of the organoid culture system 110. The organoid 108 may be digested out of the scaffold material 114 and resuspended in a hydrogel (e.g., fibrin hydrogel). In some implementations, the organoid 108 is subsequently cryopreserved and thawed.

Once generated, the organoid 108 (or multiple organoids 108) can be used for any of a variety of different applications. In some cases, organoid 108 is used to treat a patient 116. For instance, the organoid 108 is a graft that is implanted (i.e., grafted) into the patient 116. As used herein, the term “graft,” and its equivalents, can refer to an organoid, numerous organoids, and/or a piece of living tissue that is suitable for surgical transplant. In some implementations, multiple organoids 108, which may be generated using cells obtained from one or more adult donors 106, are generated and implanted into the patient 116.

In various cases, the organoid 108 is orthotopically or ectopically transplanted into the patient 116. For instance, the organoid 108 is transplanted into the patient 116 by injecting the organoid(s) 108 into the spleen or into the liver, by attaching the organoid 108 to existing liver tissue in the patient 116, by connecting the organoid 108 to at least one of the hepatic artery, hepatic portal vein, the common bile duct, another type of hepatic vasculature of the patient 116, gonadal fat pad, or any other ectopic site (e.g., lymph node), or any combination thereof.

In some cases, the organoid 108 is used to generate an engineered liver tissue or engineered liver 120. For example, the organoid 108 is seeded or encapsulated within a secondary scaffold material into an engineered liver tissue or engineered liver 120 that can be used to augment or replace at least one function associated with native liver, such as in the patient 116. In some cases, the secondary scaffold material includes one or more materials suitable for the scaffold material 114 described above. For instance, the secondary scaffold material includes a natural and/or biological extracellular matrix or a synthetic extracellular matrix.

In some cases, the organoid 108 is further grown into an engineered liver 120 that can be used to augment or replace at least one function associated with native liver, such as in the patient 116. As used herein, the terms “engineered liver,” “engineered liver tissue,” and their equivalents, can refer to an entire liver or liver tissue that includes a portion of an entire liver. In various cases, the engineered liver 120 more closely conforms to the shape and function of a mature liver than the organoid 108. In some implementations, the engineered liver 120 is generated by growing the organoid 108 to incorporate non-hepatocyte liver cell types (e.g., hepatic stellate cells, Kupffer cells, liver sinusoidal endothelial cells, etc.) in addition to the hepatocytes 102, such that the engineered liver 120 can replicate at some functions of mature liver tissue. In some examples, exogenous fibroblasts or other cell populations are added to the organoid 108. According to various cases, the engineered liver 120 is generated by supplying oxygen to cells within the organoid 108 using blood flow. For instance, artificial blood vessels and/or natural blood vessels may supply blood to cells within the organoid 108 as the organoid 108 develops into the engineered liver 120. In various implementations, bioprinting or other biofabrication techniques are used to generate the engineered liver 120. For example, the shape of the scaffold material 114 in the organoid culture system 110 and/or the arrangement of the hepatocytes 102 on and/or in the scaffold material 114 can be optimized to promote the hepatocytes 102 to grow in a complex 3D structure that enables the organoid 108 to develop into an engineered liver 120. In some cases, the engineered liver 120 is directly transplanted into the patient 116.

According to various implementations, the organoid 108 and/or engineered liver 120 is used for drug development. For example, the organoid 108 and/or engineered liver 120 can be exposed to a candidate therapeutic. The candidate therapeutic can be evaluated based on the reaction of the organoid 108 and/or engineered liver 120 to the candidate therapeutic. For instance, the toxicity of the candidate therapeutic can be assessed based on an amount of the cells in the organoid 108 and/or engineered liver 120 that die in response to being exposed to the candidate therapeutic. In some implementations, the metabolism of the candidate therapeutic is evaluated based on an amount of at least one molecule produced by the organoid 108 and/or engineered liver 120 in response to being exposed to the candidate therapeutic. In some cases, the candidate therapeutic is presumed to treat a liver-related condition. Accordingly, in some implementations, the efficacy of the candidate therapeutic is determined based on the response of the organoid 108 and/or engineered liver 120 to the candidate therapeutic. In various implementations, multiple organoids 108 and/or engineered livers 120 can be generated to screen multiple candidate therapeutics in parallel. In some cases, a single organoid 108 and/or engineered liver 120 is exposed to multiple candidate therapeutics, such that interactions between the multiple candidate therapeutics can be observed.

The organoid 108 and/or engineered liver 120 can be used for infectious disease applications. In various examples, the organoid 108 and/or engineered liver 120 can be exposed to an infectious agent (e.g., a virus, a bacteria, a eukaryotic parasite, etc.) that is known to cause illness. Techniques for treating and/or managing the illness can be developed by observing and manipulating the organoid 108 and/or engineered liver 120 in vitro, without causing harm to human subjects.

In some cases, the organoid 108 and/or engineered liver 120 is generated to develop a liver-related disease as a model system. For example, cells in the organoid 108 and/or the engineered liver 120 manifest nonalcoholic fatty liver disease (NAFLD), hepatocellular carcinoma, alpha-1 antitrypsin (A1AT) deficiency, tyrosinemia, or hemophilia. In some implementations, the adult donor 104 of the hepatocytes 102 has the liver-related disease. Techniques for managing and/or curing the liver-related disease can be developed using the organoid 108 and/or the engineered liver 120.

Although various descriptions of applications of organoids, such as the organoid 108, are described using a singular organoid 108, implementations are not so limited. For instance, an individual can be implanted with multiple organoids including the organoid 108, which may be obtained using cells from one or more donors. In some cases, the single organoid 108 is generated using cells (e.g., hepatocytes) obtained from multiple donors including the adult donor 104. Furthermore, hepatocytes 102 obtained from the adult donor 104 can be used to generate multiple organoids, such as the organoid 108.

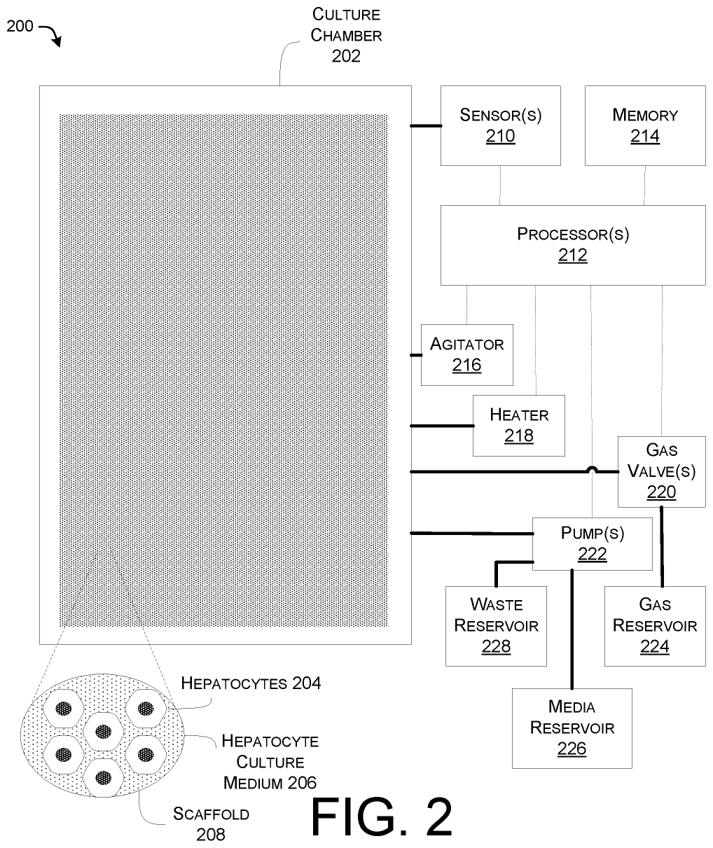

FIG. 2 illustrates an example system 200 for generating organoids. The system 200, for instance, is and/or includes the organoid culture system 110 described above with reference to FIG. 1. In particular cases, the system 200 includes one or more microfluidic devices, spinning flasks, tissue culture plates, and/or bioreactors.

The system 200 includes a culture chamber 202 that contains hepatocytes 204 and hepatocyte culture medium 206. In some cases, the example system 200 also contains a scaffold material 208 and/or other cell populations. In various cases, the hepatocytes 204 are obtained from one or more adult donors.

Various sensors 210 are configured to detect one or more parameters indicative of the environment within the culture chamber 202. For example, the sensor(s) 210 include temperature sensors, pH sensors, gas sensors (e.g., carbon dioxide sensors), image sensors (e.g., a camera), motion sensors (e.g., an accelerometer), pressure sensors, or any combination thereof. In some examples, the sensor(s) 210 include one or more analog-to-digital converters (ADCs) configured to convert one or more analog signals indicative of the parameter(s) (e.g., electrical signals generated by the sensor(s) 210) into digital data. In various implementations, the sensor(s) 210 output digital data indicative of the parameter(s) to one or more processor(s) 212. In some embodiments, the processor(s) 212 includes a Central Processing Unit (CPU), a Graphics Processing Unit (GPU), or both CPU and GPU, or other processing unit or component known in the art.

The processor(s) 212 are configured to execute instructions stored in memory 214. In various embodiments, the memory 214 is volatile (such as RAM), non-volatile (such as ROM, flash memory, etc.) or some combination of the two. The memory 214 may store, or otherwise include, various components. In some cases, the components can include objects, modules, and/or instructions to perform various functions disclosed herein. The components can include methods, threads, processes, applications, or any other sort of executable instructions. The components can include files and databases.

In particular implementations, the processor(s) 212 are configured to control one or more actuators that effect the environment within the culture chamber 202. These actuator(s) include, for instance, an agitator 216, a heater 218, one or more gas valves 220, and one or more pumps 222. In particular cases, the processor(s) 212 cause the actuator(s) to maintain conditions that are conducive to culturing the hepatocytes 204 and generating an organoid from the hepatocytes 204. The processor(s) 212 control the actuator(s) based on the parameter(s) detected by the sensor(s) 210. In various implementations, the processor(s) 212 cause the actuator(s) to maintain a predetermined range of temperature, pH, gas level, motion, or pressure within the culture chamber 202.

For example, the processor(s) 212 control the agitator 216 to establish a predetermined flow condition within the culture chamber 202. The agitator 216 may include a motor mechanically coupled to an impeller, wherein the motor is configured to move the impeller within the culture chamber 202. In some cases, the agitator 216 includes a motor mechanically coupled to a support that is configured to move (e.g., rotate) the culture chamber 202. In some cases, the agitator 216 includes a source of gas configured to generate bubbles within the culture chamber 202, or one or more pumps that generate flow within the culture chamber 202. In various implementations, the agitator 216 mixes the environment within the culture chamber 202, thereby dispersing materials, gasses, heat, and other factors within the culture chamber 202.

The processor(s) 214 control the heater 218 to maintain a temperature within the culture chamber 202. In various cases, the heater 218 includes one or more heating elements configured to produce heat and/or to reduce the temperature of the culture chamber 202. For instance, the heater 218 includes one or more resistive heating elements, one or more Peltier elements, or the like. The heating element(s) may be physically coupled to the culture chamber 202, disposed at a distance from the culture chamber 202, disposed within the culture chamber 202, disposed outside of the culture chamber 202, or any combination thereof.

The processor(s) 214 control the gas valve(s) 220 to maintain a pressure and/or gas condition within the culture chamber 202. The gas valve(s) 220 are fluidly connected to the sample chamber 202 and a gas reservoir 224. In various implementations, the gas valve(s) 220 further include a vent to an external environment. The gas valve(s) 220, for example, include at least one of a gate valve, a butterfly valve, or a ball valve. In some cases, the gas reservoir 224 contains oxygen, nitrogen, carbon dioxide, or any combination thereof.

The processor(s) 214 control the pump(s) 222 to selectively renew the hepatocyte culture medium 206 and remove waste product form the culture chamber 202 as the organoid is generated. The pump(s) 222 are fluidly connected to the culture chamber 202, a medium reservoir 226, and a waste reservoir 228. The processor(s) 214 selectively activate the pump(s) 222 to move hepatocyte culture medium from the medium reservoir 226 into the culture chamber 202 and/or to move waste from the culture chamber 202 to the waste reservoir 228. The pump(s) 222, in various cases, include at least one peristaltic pump, at least one pressure-driven flow control pump, or any combination thereof. In various cases, the processor(s) 214 cause the pump(s) 222 to replace the hepatocyte culture medium 206 within the culture chamber 202 every 12 hours, every day, every two days, every three days, or every four days.

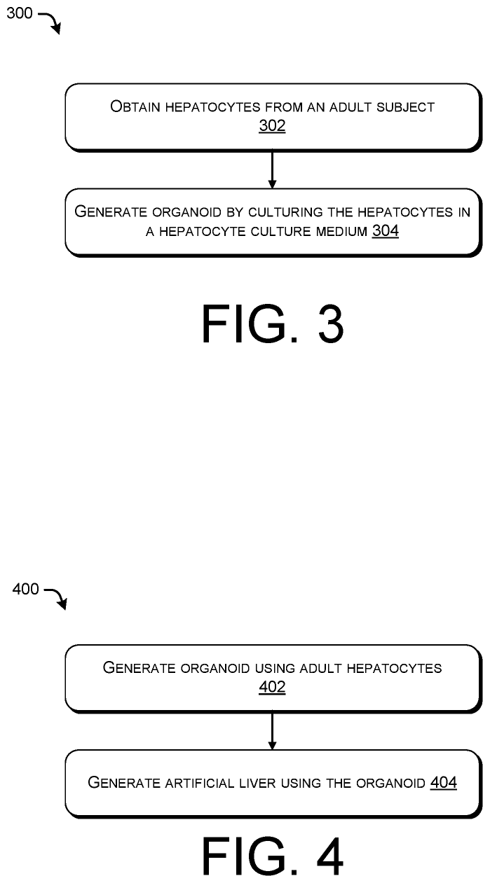

FIG. 3 illustrates an example process 300 for generating an organoid using adult hepatocytes. The example process 300 is performed by an entity, such as a device (e.g., the organoid culture system 110 and/or system 200) and/or user.

At 302, the entity obtains hepatocytes from an adult subject. In some implementations, the hepatocytes are obtained from a biopsy of the liver or digested from a whole liver of a living or deceased adult individual. In some implementations, the hepatocytes are at least temporarily cryopreserved and later thawed.

At 304, the entity generates an organoid by culturing the hepatocytes in a hepatocyte culture medium. The hepatocyte culture medium is an aqueous solution that supports hepatocyte growth. In some cases, the hepatocytes are cultured in an automated cell culture system, such as a bioreactor, in a well plate, or some other cell culture container. Various components within the hepatocyte culture medium may vary over time.

In various implementations, the hepatocyte culture medium includes a basal medium. The basal medium may include various nutrients that support hepatocyte culture, such as carbohydrates (e.g., glucose), amino acids, peptides, proteins, fats, or any combination thereof. In various cases, the basal medium includes DMEM and/or F-12. In some examples, the basal medium includes at least one of HEPES, penicillin, streptomycin, nicotinamide, N-acetyl cysteine, gastrin, or another type of cell culture supplement. In particular cases, the basal medium includes 1× GlutaMAX, 1-5% B27, 5-10 mM nicotinamide, 1-1.5 mM N-acetyl cysteine, and 5-15 nM gastrin. In some cases, the basal medium is 30-50% of the hepatocyte culture medium.

Further, the hepatocyte culture medium may include a TGF-β inhibitor at a relatively low concentration. For instance, the TGF-β inhibitor is included at a concentration of 0.1-20 μM, such as a concentration of 5 μM. The TGF-β inhibitor, for instance, is A83-01.

In some implementations, the hepatocyte culture medium includes other components, such as EGF, a non-canonical Wnt signaling potentiator, a canonical Wnt potentiator, a ROCK inhibitor, a bile acid, a bile salt, OSM, TGF-α, IGF, VEGF, IGF-I, IGF-II, PGE-2, IL1-β, a Notch ligand, Ang-2, Activins, a β-catenin pathway signaling potentiator, CHIR99021, HGF, FGF, TNF-α, an interleukin (e.g., IL6), OSM, insulin, norepinephrine, glucagon, dexamethasone, N-acetyl cystine, or glucose. For instance, the hepatocyte culture medium includes 25-1,000 ng/mL EGF, 5-15% by weight of the non-canonical Wnt signaling potentiator, 40-60% by weight of the canonical Wnt signaling potentiator, 5-10 uM of the ROCK inhibitor, 1-100 nM of FGF, 10-100 nM TNF-α, or any combination thereof. In various implementations, the non-canonical Wnt signaling potentiator includes Rspondin1 (e.g., recombinant protein) and/or the canonical Wnt signaling potentiator includes Wnt3a (e.g., recombinant protein). According to some examples, the ROCK inhibitor includes y-27632. In some implementations, the hepatocyte culture medium includes at least one molecule that stimulates one or more pathways, such as an EGF pathway, an HGF pathway, an FGF pathway, or a TNF-α pathway. In some cases, the hepatocyte culture medium includes at least one molecule that stimulates a pathway, such as a TGF-β pathway.

In various instances, the hepatocytes are further cultured in the presence of a scaffold material. For example, the scaffold material includes at least one of a solubilized basement membrane (e.g., MATRIGEL®), collagen, fibrin, vitronectin, GelMA, PEGDA, gelatin methacrylate, PEG, or a synthetic extracellular matrix.

Once generated, the organoid may exhibit one or more functions similar to mature liver tissue. For example, the organoid expresses one or more metabolic genes. In some cases, the organoid expresses one or more lipid-metabolizing genes, such as APOA2, APOC1, or FABP1. In some implementations, the organoid expresses one or more amino acid-metabolizing genes, such as GAMT, MGST1, or HPD. In some examples, the organoid expresses one or more drug-metabolizing genes, such as CYP3A4, CYP2E1, or CES1. In various cases, the organoid includes cells (e.g., at least a threshold percentage of cells) that do not express one or more fetal genes, such as at least one of AFP, CYP3A7, H19, GPC3, DLK1, or SPINK1. In some implementations, the organoid expresses one or more plasma protein genes, such as at least one of ALB, TTR, or RBP4. The organoid, for instance, secretes albumin and/or A1AT.

In various implementations, the hepatocytes in the organoid express one or more genetic diseases. For instance, the adult donor and/or the organoid express NAFLD, hepatocellular carcinoma, A1AT deficiency, tyrosinemia, or hemophilia.

FIG. 4 illustrates an example process 400 for implanting an adult hepatocyte organoid in a subject. The example process 400 is performed by an entity, such as a device (e.g., the organoid culture system 110 and/or system 200) and/or user.

At 402, the entity generates an organoid using adult hepatocytes. In various implementations, the adult hepatocytes are differentiated liver cells obtained from an adult donor. Optionally, the adult hepatocytes are cryopreserved and thawed. The adult hepatocytes, for instance, are cultured in the presence of a hepatocyte culture medium and/or a scaffold material. In various cases, the organoid exhibits one or more functions similar to mature liver tissue.

At 404, the entity generates an artificial liver using the organoid. In some implementations, the organoid is grafted onto a portion of an organism, such that the organism supplies blood to the organoid as the organoid develops into the artificial liver. In various cases, the organoid is fabricated, injected, and/or bioprinted into a 3D structure that supports delivery of oxygenated blood to the cells within the organoid. In various examples, additional cell types are introduced and/or grown with the organoid, such that the artificial liver develops with the hepatocytes from the organoid as well as other cell types.

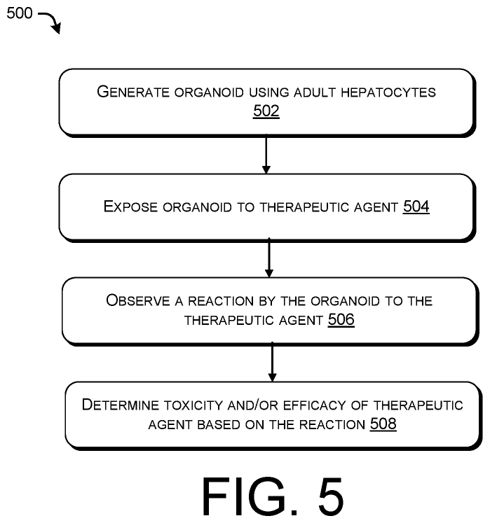

FIG. 5 illustrates an example process 500 for evaluating a therapeutic agent using an adult hepatocyte organoid. The example process 500 is performed by an entity, such as a device (e.g., the organoid culture system 110 and/or system 200) and/or user.

At 502, the entity generates an organoid using adult hepatocytes. In various implementations, the adult hepatocytes are differentiated liver cells obtained from an adult donor. Optionally, the adult hepatocytes are cryopreserved and thawed. The adult hepatocytes, for instance, are cultured in the presence of a hepatocyte culture medium and/or a scaffold material. In various cases, the organoid exhibits one or more functions similar to mature liver tissue. In some instances, multiple organoids are generated using adult hepatocytes obtained from one or more adult donors.

At 504, the entity exposes the organoid to a therapeutic agent. In various cases, the therapeutic agent is a small molecule and/or biomolecule with a potential therapeutic use. In some examples, a solution including the therapeutic agent is introduced to a medium in which the organoid is cultured (e.g., the hepatocyte culture medium or another medium in which the organoid has been transferred). In some cases, the therapeutic agent is introduced to a blood vessel supplying blood to the organoid. In various implementations, the therapeutic agent is exposed to multiple organoids generated at 502.

At 506, the entity observes a reaction by the organoid to the therapeutic agent. In various cases, the organoid is observed visually. For instance, a camera obtains one or more images of cells within the organoid periodically after the therapeutic agent is introduced. In some cases, metabolites of the cells within the organoid are tested and/or monitored over time. In some cases, metabolites of the cells or functions of the organoid (or organoids) are tested and/or monitored over time using genetic or synthetic biological constructs, such as reporter systems.

At 508, the entity determines a toxicity and/or efficacy of the therapeutic agent based on the reaction. In various implementations, the therapeutic agent may be determined to be toxic if the entity observes that the therapeutic agent induces significant cell death, or one or more metabolites associated with a toxicity response, from the organoid. In some cases, the therapeutic agent may be designed to target a problem expressed by the organoid, such as a genetic disease or physical injury. The entity may determine that the therapeutic agent is effective if the problem with the organoid is at least partially resolved in response to the introduction of the therapeutic agent.

### First Experimental Example

The human adult liver performs hundreds of life-sustaining functions. Currently, adult human liver organoid cultures can be generated from biliary epithelial cells, but not from adult hepatocytes that perform most of the mature liver's functions. Described herein is the establishment of an organoid system that supports culture of adult human hepatocytes from at least eight different adult donors. These organoids accurately model the phenotype and morphology of hepatocytes in the mature liver, express an adult liver transcriptome distinct from fetal hepatocytes, and exhibit mature functions such as inducible cytochrome activity. Adult human hepatocyte organoids efficiently engraft in vivo in engineered tissues, exhibiting up to 25-fold greater functionality after implant compared to previous hepatocyte models. Furthermore, organoids can negate the need for inclusion of exogenous non-parenchymal cells in engineered tissues to support engraftment, an important translational milestone. These results demonstrate the potential of adult human hepatocyte organoids for diverse basic and translational applications.

### Introduction

This Example describes the generation of mature human hepatocyte organoids from eight adult donors, ages 19-49 years old and of varying sex and racial/ethnic demographics. Human hepatocyte organoids exhibited maintenance of hepatic phenotype and morphology as well as protein secretion, storage, and metabolism functions. Finally, priming human hepatocytes in organoid culture substantively enhanced hepatocyte engraftment in ectopic engineered tissues in mice with liver injury.

### Results

### Culture of Primary Adult Human Hepatocytes as Organoids

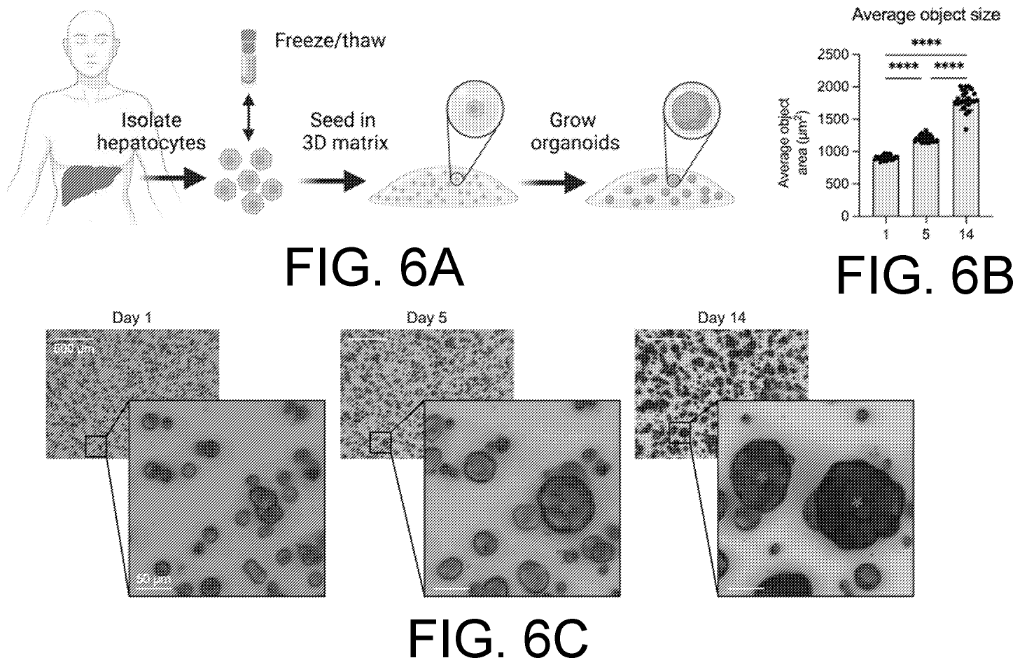

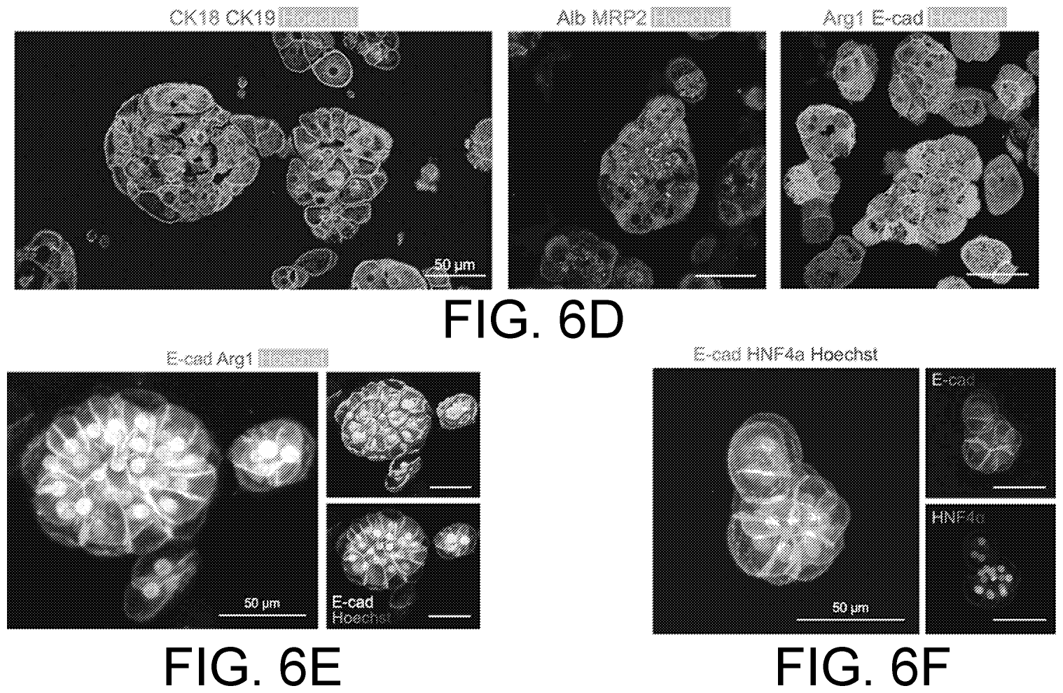

FIGS. 6A to 6F illustrate that adult human hepatocytes exhibited significant growth and maintenance of phenotype and morphology through organoid culture. FIG. 6A is a schematic illustrating the isolation, seeding, and growth of adult human hepatocytes as organoids. FIG. 6B illustrates the average cross-sectional area of all cellular and multicellular objects in individual wells on days 1, 5, and 14. Data in FIG. 6B is represented as mean+/−SEM of 23 wells, and analyzed using one-way ANOVA, Tukey's multiple comparisons test, ****p<0.0001. FIG. 6C shows representative minimum intensity projections of brightfield z-stacks used for quantification in FIG. 6B. Images were taken in the center of Matrigel droplets on days 1, 5, and 14. Asterisks track individual cells/organoids over time. In FIG. 6C, scale bars=500 μm for low magnification and 50 μm for inset images. FIG. 6D shows 2D confocal images of day 14 organoids stained for CK18 (cyan) and CK19 (magenta) (left), Alb (magenta) and MRP2 (cyan) (middle), or Arg1 (cyan) and E-cad (magenta) (right). Nuclei are stained for Hoechst (yellow) in the images shown in FIG. 6D. In FIG. 6D, the scale bars=50 μm. FIG. 6E illustrates 3D reconstructed confocal z-stack of day 14 organoids stained for E-cad (cyan), Arg1 (magenta), and Hoechst (yellow). The visualization methods used to generate FIG. 6E include maximum intensity projection (left), Imaris surface rendering of E-cad and Hoechst (top right), as well as depth color coding of Hoechst (yellow=top, pink=middle, blue=bottom) with E-cad in white (bottom right). In FIG. 6E, the scale bars=50 μm. FIG. 6F illustrates 3D reconstructed confocal z-stack of day 14 organoids stained for E-cad (cyan), HNF4α (red), and Hoechst (blue). The visualization methods used to generate FIG. 6F include maximum intensity projection (left), depth color coding of E-cad (top right), and HNF4α (bottom right) (yellow=top, pink=middle, blue=bottom). In FIG. 6F, the scale bars=50 μm.

This Experimental Example shows that culturing mature human hepatocytes in a physiologically relevant 3D matrix such as Matrigel, paired with stimulation from appropriate biological factors such as liver regenerative factors, induced mature adult primary hepatocytes to form organoids (Barhouse, P. S. et al (2022). Frontiers in Chemical Engineering 0, 16) (FIG. 6A).

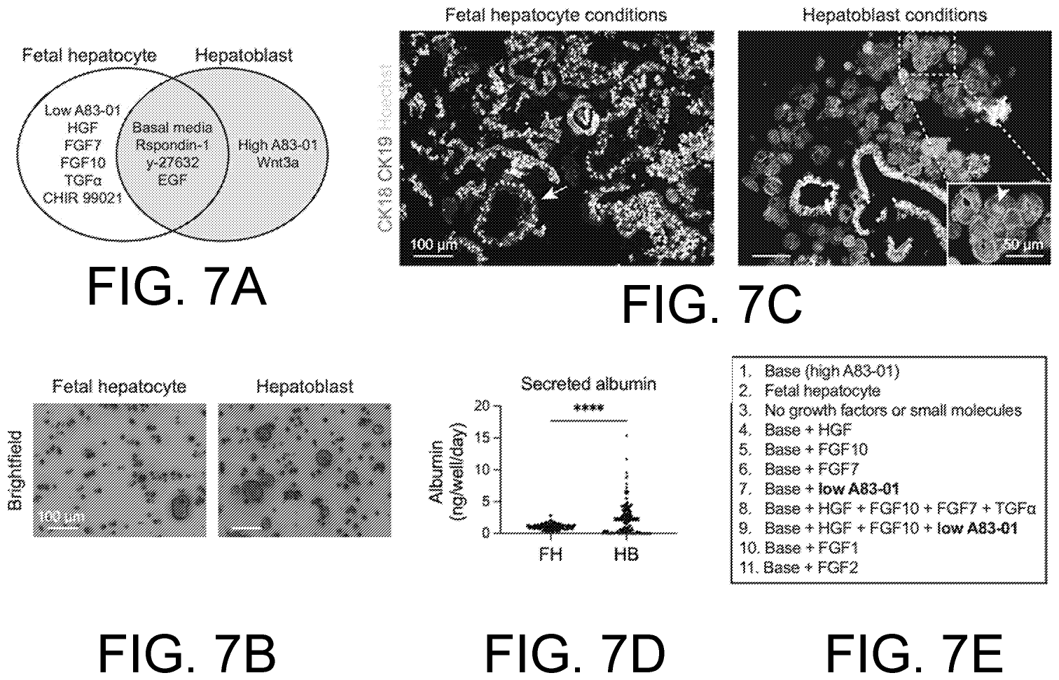

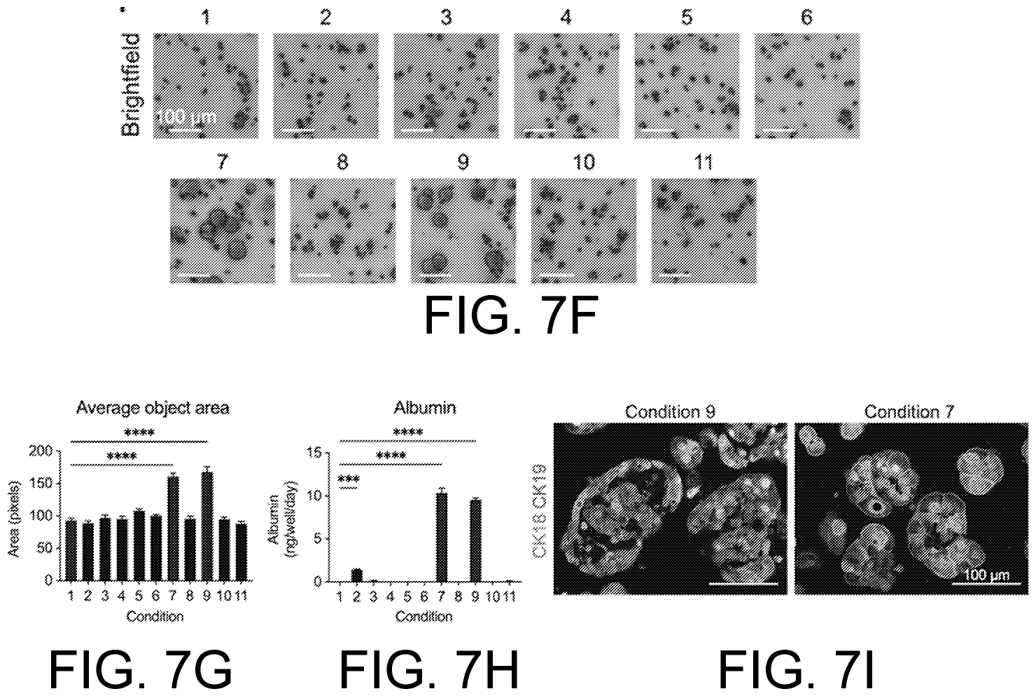

FIGS. 7A to 7I illustrate optimal adult human hepatocyte organoid culture conditions identified using in vitro screening. FIG. 7A is a venn diagram illustrating medium differences between fetal hepatocyte (left) and hepatoblast (right) organoid media. The center of the diagram shows shared medium components. FIG. 7B illustrates representative minimum intensity projections of brightfield z-stacks of adult human hepatocytes grown in fetal hepatocyte organoid conditions (left) or hepatoblast organoid conditions (right). In FIG. 7B, the scale bar=100 μm. FIG. 7C shows immunofluorescence images of adult human hepatocytes grown in fetal hepatocyte organoid conditions (left) or hepatoblast organoid conditions (right) stained for CK18 (cyan), CK19 (magenta), and Hoechst (yellow). The arrow on the left identifies an intact cystic organoid, whereas the arrowhead in the right inset indicates binucleated cell. In FIG. 7C, the scale bar=100 μm, 50 μm for the inset. FIG. 7D illustrates albumin secretion measured by human albumin enzyme-linked immunosorbent assay (ELISA) comparing adult human hepatocytes grown in fetal hepatoblast organoid conditions or hepatoblast organoid conditions. Data in FIG. 7D is represented as mean+/−SEM of 154 replicates at day 12, and was generated using Welsh's t test, ****p<0.0001. FIG. 7E shows a summary of 11 different culture conditions tested on adult human hepatocytes. The base medium includes high A83-01 (50 μM), fetal hepatocyte condition as published (Hu et al., 2018). Conditions with low A83-01 (5 μM) are shown in bold. FIG. 7F illustrates representative minimum intensity projections of brightfield z-stacks of adult human hepatocytes grown in 11 different culture conditions listed in FIG. 7E. In FIG. 7F, the scale bar=100 μm. FIG. 7G shows the average cross-sectional area of cellular and multicellular objects in a single well at day 7. In FIG. 7G, data is represented as mean+/−SEM of 16 wells, using one-way ANOVA, Dunnett's multiple comparisons test, ****p<0.0001. FIG. 7H illustrates albumin secretion measured by human albumin ELISA comparing adult human hepatocytes grown in 11 different medium conditions listed in FIG. E. Data is represented as mean+/−SEM of 4 wells at day 9, and was analyzed using one-way ANOVA, Dunnett's multiple comparisons test, ***p<0.001, ****p<0.0001. FIG. 7I shows immunofluorescence images comparing adult human hepatocyte organoids grown in condition 9 (left) and condition 7 (right) stained for CK18 (cyan), CK19 (magenta), and Hoechst (yellow). In FIG. 7I, the scale bar=100 μm.

A cocktail of growth stimuli was optimized by considering developmental hepatic organoid systems, as many important developmental signaling pathways overlap with those stimulating adult liver regeneration (Kelley-Loughnane, N. et al. (2002). Hepatology 35, 525-534, 2002; Michalopoulos, 2007; Miyajima, A. et al. (2014). Cell Stem Cell 14, 561-574; Tanimizu, N. and Miyajima, A. (2007). International Review of Cytology 259, 1-48). First, conditions previously identified to support human hepatoblast or human fetal hepatocyte organoids were tested for their organoid formation potential on previously cryopreserved primary human hepatocytes from a 34-year-old donor. Wnt and epidermal growth factor (EGF) pathways were stimulated, and rho-associated kinase (ROCK) and transforming growth factor-β (TGF-β) pathways were inhibited for both hepatoblast and fetal hepatocyte conditions (Hu et al., 2018; Wesley et al., 2022), with additional stimulation of hepatocyte growth factor (HGF), fibroblast growth factor (FGF), and transforming growth factor-α (TGF-αTGF-α) for fetal hepatocyte culture conditions (Hu et al., 2018) (FIG. 7A). Over two weeks, single primary hepatocytes seeded in Matrigel formed into small organoids under both culture conditions (FIG. 7B), though immunostaining revealed that nearly all cells grown under fetal hepatocyte conditions expressed cytokeratin-19 (CK19), a cytoskeletal protein strongly expressed by cholangiocytes and some hepatic progenitor cells, but generally absent in healthy mature hepatocytes (FIG. 7C, left). Furthermore, many of the CK19+ organoids grown under fetal hepatocyte culture conditions had organized into a single or double layer of small cells forming a hollow cystic structure, a morphology typical of liver ductal cell or cholangiocyte organoids (Huch et al., 2015; Sampaziotis et al., 2017) (FIG. 7C, left, arrow). Organoids grown under hepatoblast culture conditions also displayed ductal morphology, though dense organoids with strong expression of epithelial marker cytokeratin 18 (CK18) and low expression of CK19 were also present (FIG. 7C, right). Immunostaining demonstrated that cells in dense organoids displayed hepatocyte morphological signatures such as a low nuclear-to-cytoplasmic ratio, strong CK18 expression at cell-cell junctions, and some binucleated cells (FIG. 7C, right, arrowhead). Medium supernatants from organoids cultured under each condition were also analyzed for the presence of human albumin, a protein produced in abundance by human hepatocytes, and thus a first-pass surrogate for querying the presence of functional hepatocytes. Human albumin secretion was consistently lower across wells grown in fetal hepatocyte conditions, as compared to those grown in hepatoblast conditions, which had significantly higher albumin production, on average. However, albumin levels varied widely from well-to-well, with some containing no measurable albumin (FIG. 7D). While the presence of occasional organoids with aspects of hepatocyte phenotype in hepatoblast culture conditions was encouraging, the phenotypic and functional consistency across cultures remained low. Thus, culture conditions were next optimized to achieve human hepatocyte organoid cultures with high purity, functionality, and well-to-well consistency across cultures.

To do this, hepatoblast medium was further supplemented with a portfolio of additional factors implicated in liver development and regeneration, including growth factors HGF, FGF1, FGF2, FGF7, FGF10, TGF-α, and the small molecule A83-01 (FIG. 7E). After one week, brightfield imaging identified two conditions that robustly supported gross organoid growth and morphology (FIG. 7F; conditions 7 and 9). This observation was confirmed with morphometric analysis of brightfield images showing a significant increase in average object area, a metric of organoid size (FIG. 7G). Organoids cultured under these two conditions also had significantly higher human albumin secretion compared to all other conditions screened (FIG. 7H). Notably, compared to all others tested, both of these conditions had a lower concentration of A83-01, a small molecule that blocks TGFβ signaling through inhibition of TGFβ receptor 1.

Of the two conditions with low A83-01, one (condition 9) was additionally supplemented with HGF and FGF10 and generated organoids that demonstrated intermediate or mixed phenotypes, with the majority of organoids exhibiting dense morphology but heterogenous CK19 expression (FIG. 7I, left). Conversely, organoids from the second condition (condition 7, with no HGF or FGF10) demonstrated a more consistent morphology in which organoids were predominantly dense and contained CK18+/CK19− cells (FIG. 7I, right). Due to this phenotypic conservation paired with improved organoid formation and high function, condition 7 was used to culture adult human hepatocytes as organoids for all subsequent phenotypic and functional analyses. Thus, condition 7 is subsequently described within this Experimental Example as “mature human hepatocyte culture conditions.”

Next, the growth and phenotype of human hepatocytes cultured under mature human hepatocyte culture conditions were characterized. To do this, human hepatocytes were grown from a 19-year-old donor as organoids and then the cross-sectional area of cellular or multicellular objects in brightfield images were measured over time.

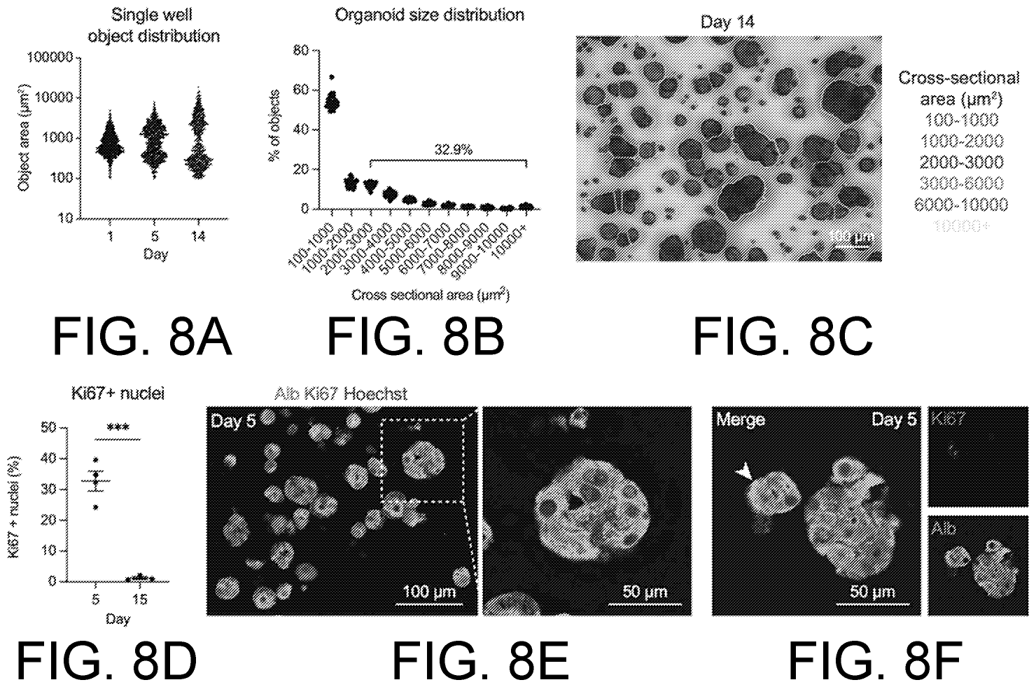

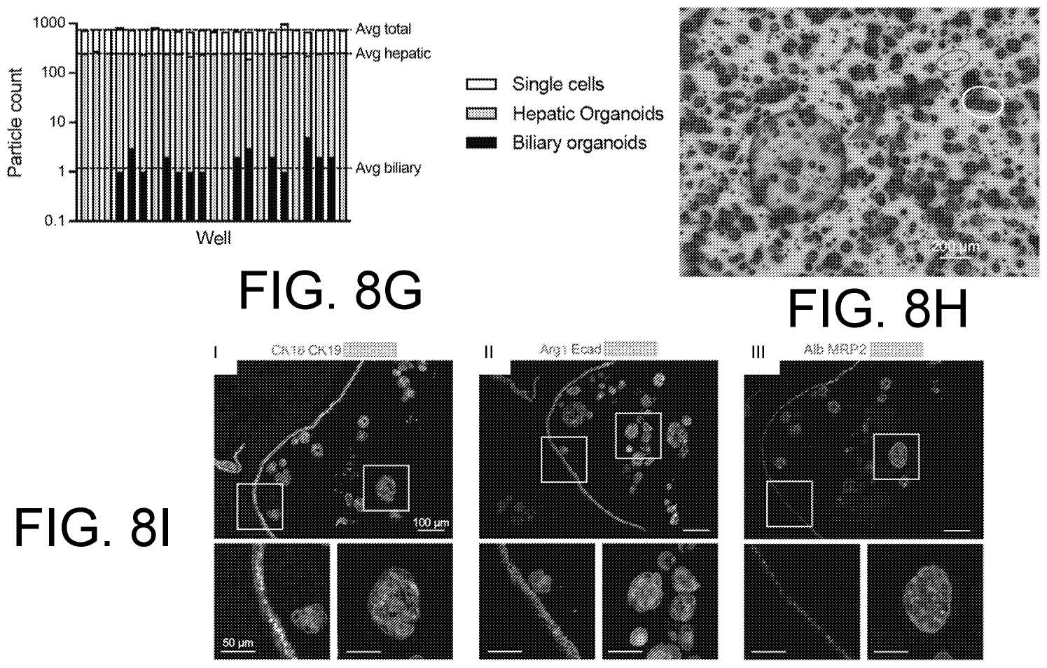

FIGS. 8A to 8I illustrate adult human hepatocyte organoid growth tracking and phenotypic characterization. FIG. 8A shows the cross-sectional area of all cellular/multicellular objects in a single well at day 1, 5, and 14. Each dot represents a single object. FIG. 8B illustrates the distribution of object sizes at day 14 across 24 wells. Each dot represents the percentage of organoids in that size range in a single well. On average 32.9% of objects were larger than 2000 μm² and thus were considered organoids. FIG. 8C shows minimum intensity projection of brightfield z-stack demonstrating color-coded cross-sectional area ranges used for quantification in FIG. 6B and FIGS. 8A and 8B. In FIG. 8C, the scale bar=100 μm. FIG. 8D shows the quantification of Ki67+ nuclei in Alb+ cells at days 5 and 15. Data is represented as mean+/−SEM of 3 or 4 replicates, using the unpaired t test, ***p<0.001. FIG. 8E shows an immunofluorescence image of day 5 organoid sample stained for Alb (cyan), Ki67 (red), and Hoechst (blue). In FIG. 8E, the scale bar=100 μm for the low magnification image, 50 μm for the inset image. FIG. 8F shows a fluorescence image demonstrating mitotic figure (arrowhead) in day 5 organoid stained for Alb (cyan), Ki67 (red), and Hoechst (blue). Image shown merged (left) and with Ki67 and Alb channels separated (right). In FIG. 8F, the scale bar=50 μm. FIG. 8G shows a distribution of cellular morphologies across 24 wells. Biliary organoids (cystic morphology, pink bars) make up an average of 0.16% of all cellular objects. Hepatic organoids (dense morphology, area >2000 μm², blue bars) make up an average of 32.6% of all cellular objects. Single/double cells (dense morphology, area <2000 μm², grey bars) make up remaining 67.2% of all cellular objects. FIG. 8H shows a minimum intensity projection of a brightfield z-stack demonstrating cellular morphologies used for quantification in FIG. 8G. An arrow indicates a biliary organoid with cystic morphology. A white circle indicates a cluster of hepatic organoids with dense morphology. A gray circle indicates single/double cells. In FIG. 8H, the scale bar=200 μm. FIG. 8I illustrates fluorescence images of serial day 14 organoid sections stained for (I) CK18 (cyan) and CK19 (magenta), (II) Arg1 (cyan) and Ecad (magenta), and (III) Alb (magenta) and MRP2 (cyan). Nuclei stained for Hoechst (yellow) in all images. Left insets demonstrate a cystic biliary organoid, right insets demonstrate dense hepatic organoid. In FIG. 8I, the low magnification scale bar=100 μm, and the inset scale bars=50 μm.

On the first day after cell seeding, the average area of all objects measured was 907 μm² (estimated ˜34 μm diameter, assuming sphericity) with a normal distribution, reflecting a relatively homogenous population of single hepatocytes or small clumps of a few cells (FIGS. 6B, 6C, and 8A). By day 5, however, the cell population had begun to separate into a bimodal distribution, with a lower mode representing inert single cells and an upper mode representing growing organoids. The distribution further stratified by day 14, with ˜⅓ of objects forming organoids (FIGS. 8A-C) that ranged from ˜50 μm to over 150 μm in diameter.

To further investigate the phenotypic identity of the cells comprising the organoids, immunostaining for various structural and functional hepatic proteins was performed. Immunostaining for CK18 demonstrated that cells self-organized into densely packed spheroids with large cells and a low nuclear to cytoplasmic ratio, similar to primary hepatocytes (FIG. 6D, left). Cells in adult human hepatocyte organoids were largely negative for the biliary/progenitor cell marker CK19, though occasional cells were found that were CK19+, but generally maintained hepatic as opposed to biliary morphology (FIG. 6D, left). The majority of cells expressed albumin (Alb), a marker of functional hepatocytes, and arginase-1 (Arg1), an enzyme involved in nitrogen metabolism (FIG. 6D, center and right). Adult human hepatocyte organoids also expressed the cell-cell junction protein E-cadherin (E-cad), which was primarily located at the cell membrane and between cells (FIG. 6D, right). Multidrug resistance-associated protein 2 (MRP2), an efflux transporter located in the bile canaliculi on the apical domain of polarized hepatocytes, was expressed on the interior of dense organoids (FIG. 6D, center). Samples were then immunostained for both albumin and Ki67, a protein associated with the cell cycle and thus used as a marker for proliferating cells. Ki67+ nuclei made up 34% and 1% of all albumin+ nuclei on days 5 and 15, respectively (FIGS. 8D and E). Rare Ki67+ mitotic figures were also identified, suggesting that hepatocytes in organoid culture were actively undergoing cell division (FIG. 8F). Alongside adult human hepatocyte organoids, rare and distinct cystic ductal organoids formed that were CK19, CK18, and E-cad positive, and MRP2, Alb, and Arg1 negative (FIGS. 8G-8I), but accounted for only ˜0.16% of cellular objects (FIG. 8G), possibly arising from contaminating cholangiocytes. Overall, imaging and immunostaining demonstrated that adult human hepatocytes formed organoids that grew over time in culture, and that these cells maintained robust adult primary hepatocyte morphology and phenotype.

To better assess both the 3D morphology and phenotype of cells within organoids, organoid cultures were immunostained, cleared, 3D imaged, and computationally reconstructed. These studies further demonstrated conservation of hepatocyte morphology and protein localization, with cytoplasmic Arg1 staining (FIG. 6E), nuclear expression of hepatocyte transcription factor HNF4α (FIG. 6F), and strong expression of E-cad at cell-cell junctions throughout the organoid depth and at various sizes (FIGS. 6E and 6F).

Adult Human Hepatocyte Organoids Form from a Diverse Set of Post-Adolescent Donor Hepatocytes

A confounding issue for development of adult human hepatocyte therapies and model systems has been major variability in cell viability and the functional quality of primary hepatocytes isolated from different human donors (Lin, J. H. and Lu, A. Y. H. (2001). Annu Rev Pharmacol Toxicol 41, 535-567; Mitry, R. R. et al. (2003). Cell Transplantation 12, 69-74; Shimada, T. et al. (1994). Journal of Pharmacology and Experimental Therapeutics 270). While some functional differences are expected due to natural variation in human biology (Lin and Lu, 2001; Rogue, A. et al. (2012). Drug Metab Dispos 40, 151-158; Yuan, X. et al. (2008). American Journal of Human Genetics 83, 520-528), research is often confounded by donor hepatocytes that are non-adherent or non-viable in 2D culture, or cells that cannot engraft after implant (Mitry et al., 2003; Pinkse, G. G. M. et al. (2004). Liver International 24, 218-226; Terry, C. et al. (2007). Cell Transplantation 16, 639-647). New strategies to culture adult hepatocytes from diverse samples could recover previously unusable cells and enable longer-term hepatic studies on samples with broader genetic and ancestral variation (Moore, E., et al. (2021). Nature Reviews Materials 2021 7:1 7, 2-4; Ryan, H. et al. (2021). Regenerative Engineering and Translational Medicine 1, 1). Thus, this Experimental Example illustrates that an organoid 3D culture could reliably generate adult human hepatocyte organoids from an additional set of seven diverse adult donors.

TABLE 1
Patient data for human hepatocyte lot screening

| | | | Race/ | 2D | CYP3A |
|---|---|---|---|---|---|
| Lot | Age | Sex | Ethnicity | viability* | induction* |
| 1 | 49 | F | White | 5+ | x |
| 2 | 31 | M | White | 5 days | 21.1 |
| 3 | 31 | F | Black | 5+ | 4.9 |
| 4 | 37 | F | White | 3 days | x |
| 5 | 24 | M | Hispanic | 5 days | 28.1 |
| 6 | 26 | F | White | 5 days | 34.6 |
| 7 | 19 | F | White | 5+ | 6.6 |

*= reported by Invitrogen
x = data not available

First, human donor lots with reported variation in 2D plating longevity, in which hepatocytes were viable for only 3 days, 5 days, or beyond 5 days of culture as reported by the vendor (Table 1) were identified. A subset of donors were selected from the identified human donor lots, ranging in age from 19-49 years old with varying sex (71% female, 29% male) and racial/ethnic demographics (71% White, 14% Black, 14% Latinx/Hispanic) (Table 1).

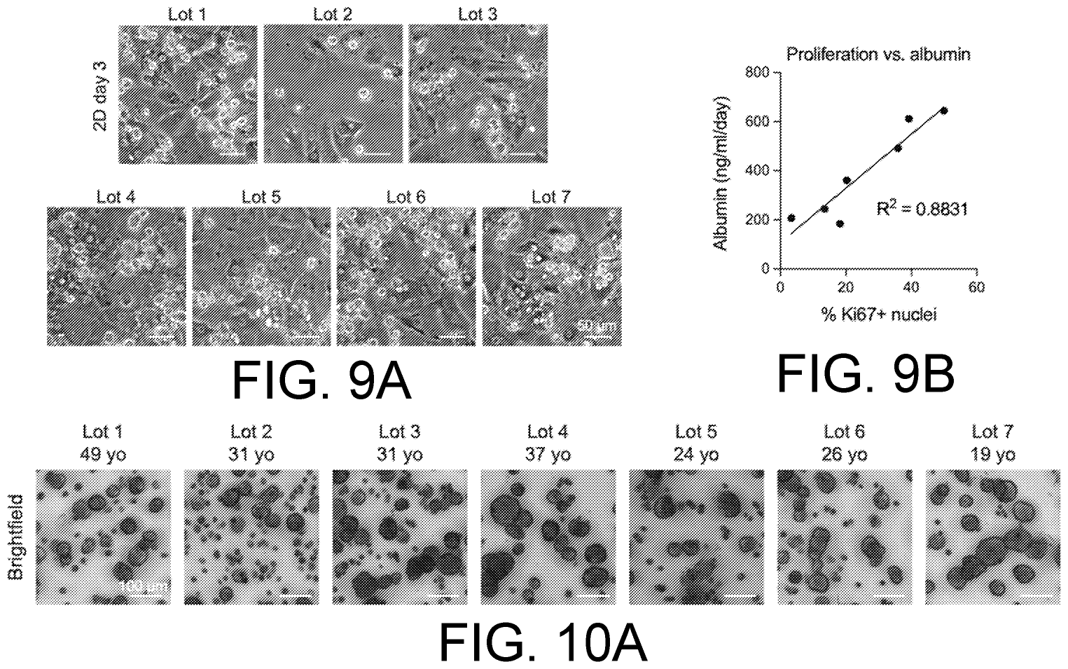

FIGS. 9A and 9B show that human hepatocytes demonstrated variability in 2D plating and albumin secretion. FIG. 9A shows representative brightfield images of cells derived from seven separate donors, Lots 1-7, in 2D culture on collagen coated plates at day 3. In FIG. 9A, the scale bar=50 μm. FIG. 9B shows a scatter plot of albumin secretion at day 15 vs percent of Ki67+ nuclei at day 5 across the 7 donor lots, and shows correlation coefficient R²=0.8831.

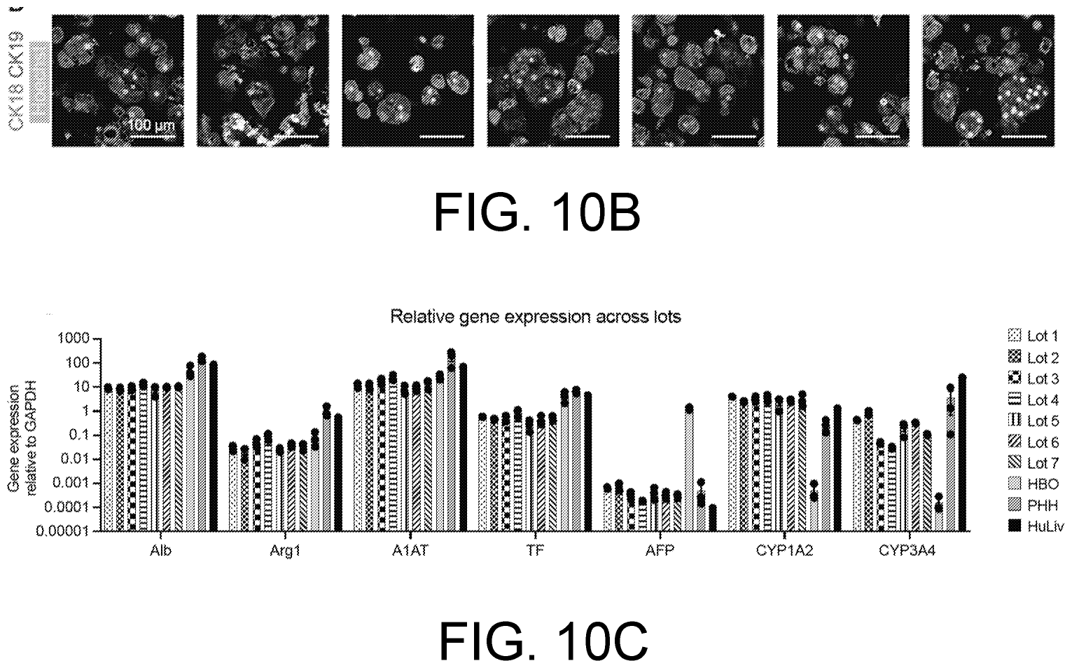

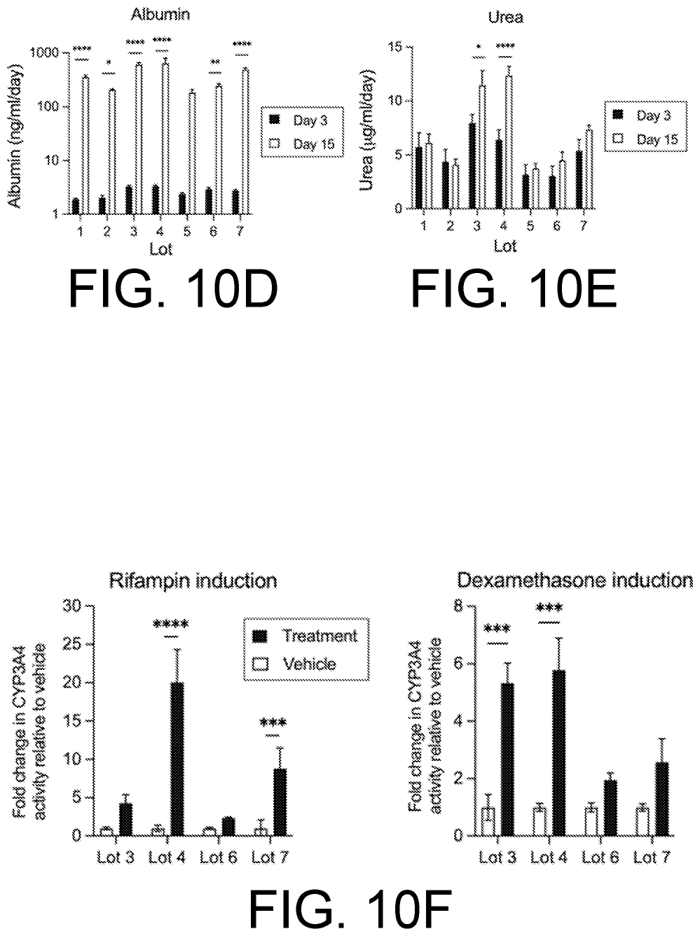

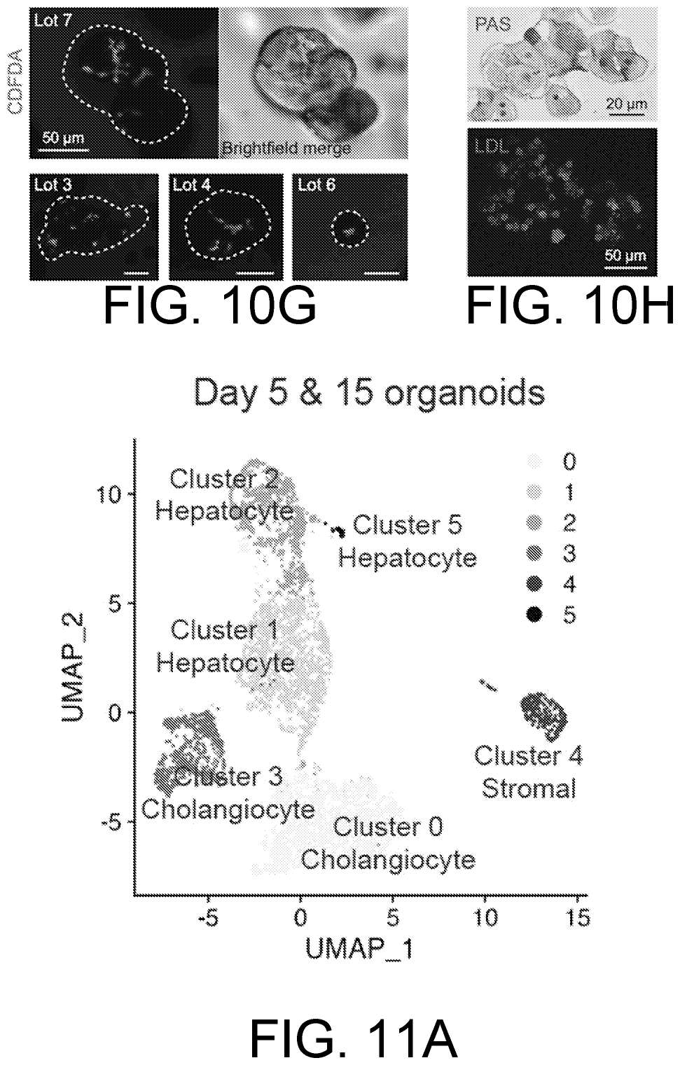

FIGS. 10A to 10H show that adult human hepatocyte organoids from seven separate human donors formed reliably and demonstrated robust hepatic functions. FIG. 10A shows representative minimum intensity projections of brightfield z-stacks across all seven lots screened with donor age listed above image. Images were taken in the center of Matrigel droplets on day 15. In FIG. 10A, the scale bars=100 μm. FIG. 10B shows immunofluorescence images across all seven lots screened stained for CK18 (cyan), CK19 (magenta), and Hoechst (yellow). In FIG. 10B, scale bars=100 μm. FIG. 10C illustrates gene expression analysis by qRT-PCR on genes of interest across all lots compared to RNA from hepatoblast organoids (HBO), freshly thawed primary human hepatocytes (PHH), and whole liver RNA (HuLiv). Gene expression is noted relative to GAPDH for each sample. Data in FIG. 10C is represented as mean+/−SEM of 2 or 3 replicates, each measured in technical triplicate. FIG. 10D shows albumin secretion across all lots measured by human albumin ELISA. Data in FIG. 10B is represented as mean+/−SEM of 8 replicates at day 3 (black) and day 15 (white), and was analyzed using two-way ANOVA, Sidak's multiple comparisons test, *p<0.05, **p<0.01, ****p<0.0001. FIG. 10E shows urea secretion across all lots measured by BUN assay. Data in FIG. 10E is represented as mean+/−SEM of 8 replicates on day 3 (black) and day 15 (white), and was analyzed using two-way ANOVA, Sidak's multiple comparisons test, *p<0.05, ****p<0.0001. FIG. 10F illustrates an analysis of CYP3A4 activity after rifampin (left) or dexamethasone (right) induction in four organoid lots by P450-Glo assay. Data in FIG. 10F is normalized to vehicle control, represented as mean fold change+/−SEM with 3 replicates each of vehicle (white) and treatment (black), and is generated using two-way ANOVA, Sidak's multiple comparisons test, ***p<0.001, ****p<0.0001. FIG. 10G illustrates confocal maximum intensity projections of CDFDA (green) accumulation in organoids, overlaid with minimum intensity projection of brightfield z-stack for lot 7 (top right). CDFDA maximum intensity projection for lots 3, 4, and 6 is displayed on the bottom of FIG. 10G. Organoids are outlined with white dashed line. In FIG. 10G, the scale bars=50 μm. FIG. 10H illustrates glycogen storage in organoids (top), visualized with PAS stain (pink) and Hematoxylin counterstain (blue) (top, scale bar=20 m); and low density lipoprotein (LDL) accumulation in organoids (bottom, scale bar=50 μm), visualized with Dil-LDL staining (red) and Hoechst (blue).

Plating efficiency in 2D was confirmed to be highly variable (FIG. 9A). Despite this variability, after plating hepatocytes from all seven donor lots for 3D culture, adult human hepatocytes formed organoids with compact hepatic morphology across all lots, as observed by brightfield morphology (FIG. 10A). Immunostaining demonstrated that organoids from all lots expressed the epithelial marker CK18 and that individual cells had adult hepatocyte morphology (FIG. 10B). Similar to other studies, cystic ductal organoids expressing CK19 were rare (<0.2%) and found in cultures for all human donor lots (FIG. 10B). Thus, adult human hepatocytes from seven additional different donors, which in 2D culture demonstrated highly variable 2D plating efficiency, successfully formed organoids when grown in mature human hepatocyte culture conditions.

Adult Human Hepatocyte Organoids from a Diverse Donor Set Maintain Range of Hepatic Functions

Although hepatocytes include a workforce that performs hundreds of functions within the human liver (Arias et al., 2020), hepatocyte functions decline rapidly in most 2D culture formats in vitro in the absence of highly engineered microenvironments, restricting their utility as a liver model (Khetani and Bhatia, 2008; Underhill and Khetani, 2018). Encouraged by the robust adult human hepatocyte organoid formation across all donor lots tested, hepatocyte functions were next assessed across the various donors. First, gene expression was assessed across different hepatocyte functional axes and adult human hepatocyte organoid gene expression was compared to hepatic cells from various stages of liver development, including human hepatoblast organoids (HBO) representing early development (Wesley et al., 2022), freshly thawed primary adult human hepatocytes (PHH), and whole adult human liver RNA (HuLiv). Expression of a panel of genes encoding secreted proteins that are upregulated in development and stay highly expressed in hepatocytes throughout adulthood (ALB, ARG1, SERPINA1, and TF (Lee, J. S. et al. (2012). BMC Genomics 13, 1-15; Wesley et al., 2022), which provide instructions for making the proteins albumin, arginase-1, alpha-1 antitrypsin, and transferrin, respectively) were assessed. Adult human hepatocyte organoids from all lots had high and remarkably similar expression for all adult protein-encoding genes (FIG. 10C). Next, genes known to be differentially expressed between fetal and adult liver (Zabulica, M. et al. (2019). Stem Cells and Development 28, 907-919) were assessed. Alpha-fetoprotein (AFP), is expressed at high levels in fetal liver, declines post-natally and is generally absent in adult hepatocytes (Lee et al., 2012; Wesley et al., 2022). As expected, human hepatoblast controls exhibited high expression of AFP (FIG. 10C). Conversely, adult human hepatocyte organoids had little to no AFP expression, similar to adult mature hepatocyte and human liver controls (FIG. 10C). Next, gene expression of two enzymes responsible for the metabolism of ˜40% of drugs in humans (cytochrome P450 genes CYP1A2 and CYP3A4 (Zanger, U. M. and Schwab, M. (2013). Pharmacology & Therapeutics 138, 103-141)) were interrogated. The majority of CYP enzymes, aside from fetal isoforms such as CYP3A7, are expressed at low levels in fetal developmental stages and increase with hepatocyte maturation (Choudhary, D. et al. (2005). Archives of Biochemistry and Biophysics 436, 50-61; Hart, S. N. et al. (2009). Three Patterns of Cytochrome P450 Gene Expression during Liver Maturation in Mice; Song, G. et al. (2017). Drug Metab Dispos 45, 468-475; Sonnier, M. and Cresteil, T. (1998). European Journal of Biochemistry 251, 893-898; Stevens, J. C. (2006). Drug Discovery Today 11, 440-445). Consistent with embryonic and fetal liver stages, the human hepatoblasts had low to no CYP1A2 or CYP3A4 expression. In contrast and importantly, adult human hepatocyte organoids prepared according to this Example exhibited high gene expression in all lots, and at similar levels as adult controls (FIG. 10C). This maintenance of mature gene expression and dearth of fetal genes demonstrates successful preservation of adult liver transcription across all lots and differentiates adult human hepatocyte organoid culture from models with an immature phenotype.

Next, a panel of functional assays was explored to assess the extent to which hepatocytes maintain important liver functions after organoid culture. Human albumin protein was measured over time to explore the secretory function of the adult human hepatocyte organoids. All lots increased albumin production as organoids grew, with an average of 150-fold increase over 2 weeks (FIG. 10D). The lots with the highest albumin secretion at day 14 were also found to have the highest percentage of hepatocytes expressing Ki67 at day 5 (correlation coefficient R2=0.8831), suggesting that the substantive increase in albumin over time was at least partly due to cell growth and organoid expansion (FIG. 9B). Synthesis of urea, a byproduct of ammonia detoxification, was also present in cultures derived from all lots, with differential patterns of urea secretion across lots mirroring trends of albumin secretion for each donor (FIG. 10E).

To assess hepatic xenobiotic metabolism, the activity of CYP3A4 (the enzyme responsible for 30-60% of drug metabolism in the human liver (Martinez-Jimenez, C. et al. (2007). Current Drug Metabolism 8, 185-194)) was explored. CYP3A4 activity can be strongly induced or inhibited by certain drugs, altering pharmacokinetics and making accurate prediction of CYP3A4 activity critical for human liver models (Lin, 2006). Adult human hepatocyte organoids from four donors were exposed to known CYP3A4 inducers rifampin and dexamethasone, and CYP3A4 activity was measured. All lots exhibited induction by both drugs, with inter-donor variability in induction levels, as would be expected for mature adult human hepatocytes in which function has been maintained (FIG. 10F). Importantly, of these, CYP3A4 induction by rifampin for lot 4 was not reported by the vendor due to its limited 2D viability, but a 20-fold induction in organoids (Table 1, FIG. 10F) was observed. Thus, the human adult human hepatocyte organoids prepared according to this Example captured the reported variability in CYP3A4 activity between donors (Lin and Lu, 2001; Shimada et al., 1994; Thummel, K. E. et al. (1994). Journal of Pharmacology and Experimental Therapeutics 271, 549-556; Wilkinson, G. R. (2005) N Engl J Med 352, 2211-2221; Yamazaki, H. et al. (2006). Xenobiotica 36, 1201-1209). This suggests that the organoids could also be used to assay CYP3A4 activity in lots that were not amenable to 2D culture.

After modifications by CYP enzymes and other biotransformations, many xenobiotics are excreted from hepatocytes by efflux transporters in the multidrug resistance protein (MRP) family (Almazroo, O. A. et al., Clinics in Liver Disease 21, 1-20; Xu, C., Li, C. Y. T. and Kong, A. N. T. (2005). Archives of Pharmacol Research 2005 28:3 28, 249-268). MRP2, a transporter that usually exports substrates from within hepatocytes out to bile canaliculi (Almazroo et al., 2017), was assessed through incubation of adult human hepatocyte organoids with fluorescent MRP2 substrate, carboxy dichlorofluorescein diacetate (CDFDA). Fluorescent signal was localized to networks or puncta on the interior of the organoids, indicating active MRP2-driven efflux and validating the morphological polarity observed with MRP2 immunostaining (FIGS. 10G and 7D). Finally, glycogen accumulation and low-density lipoprotein (LDL) uptake were observed with periodic acid Schiff (PAS) staining and Dil-LDL incubation, respectively, as assessments of hepatocyte functions (FIG. 10H). In sum, adult human hepatocyte organoids prepared from diverse donors in accordance with this Example demonstrated maintenance of a wide variety of hepatic functions, illustrating their potential as a model of functional human hepatocytes and as a tool for basic research applications and drug screening.

### Single-Cell RNA-Sequencing Reveals Transcriptional Maturity and Functional Heterogeneity in Adult Human Organoid Hepatocytes

To further explore the distribution of hepatic functions in adult human hepatocyte organoids on a cell-to-cell basis (Halpern, K. B. et al. (2017). Nature 2017 542:7641 542, 352-356), single-cell RNA sequencing (scRNAseq) was performed on the organoid population using the 10× Genomics Chromium platform.

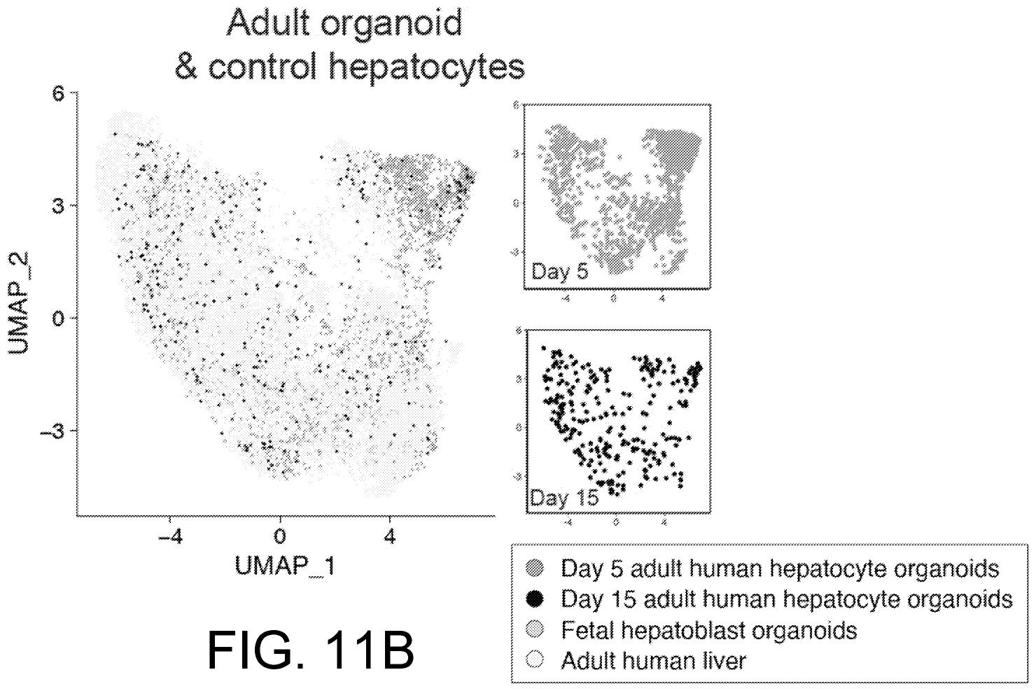

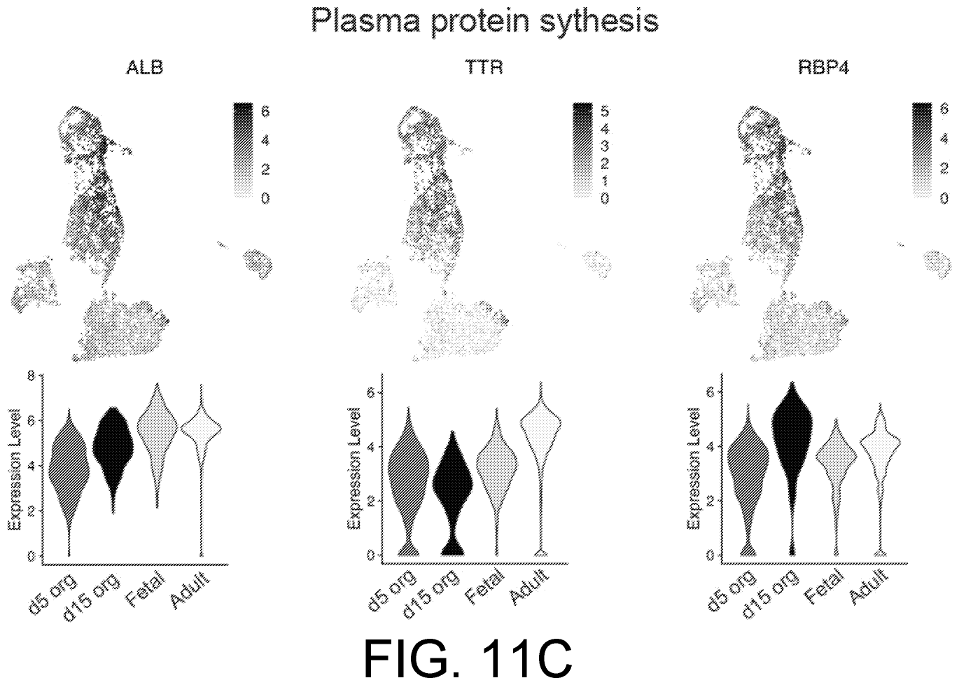

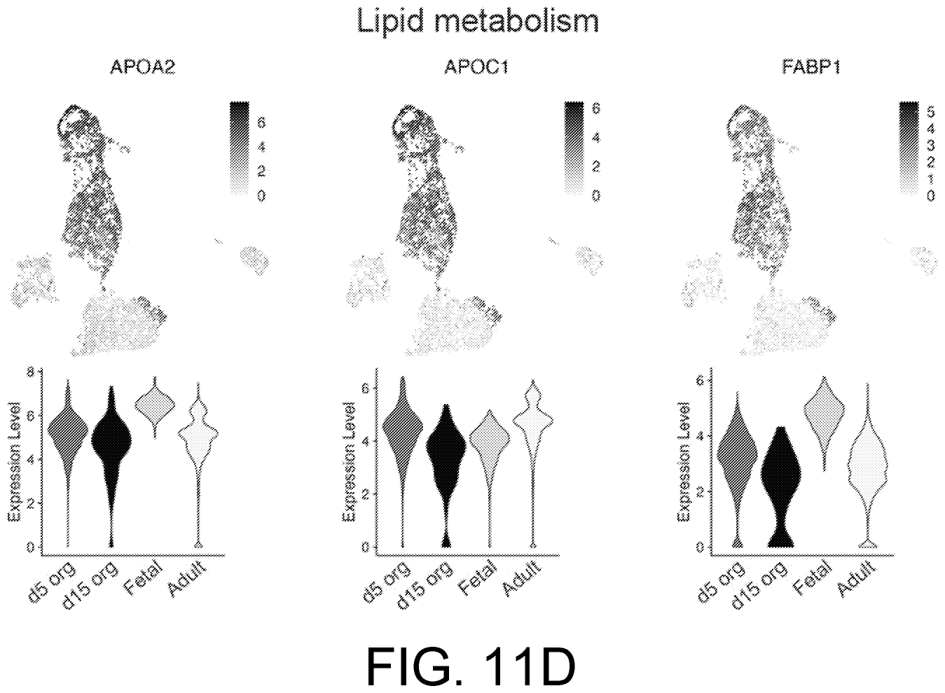

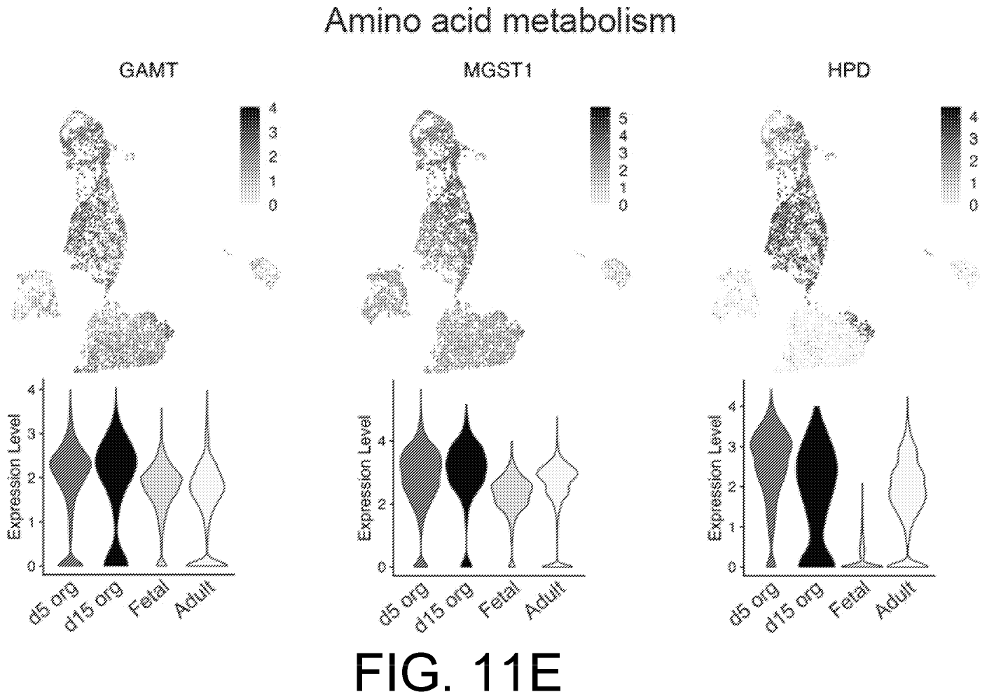

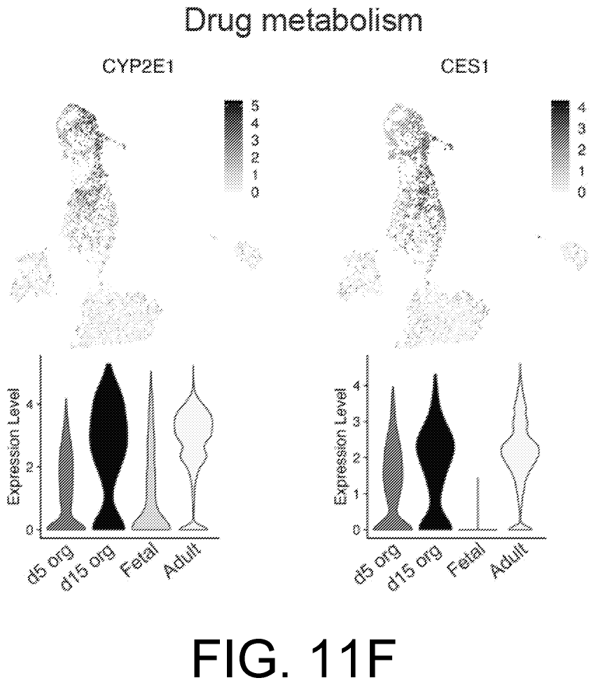

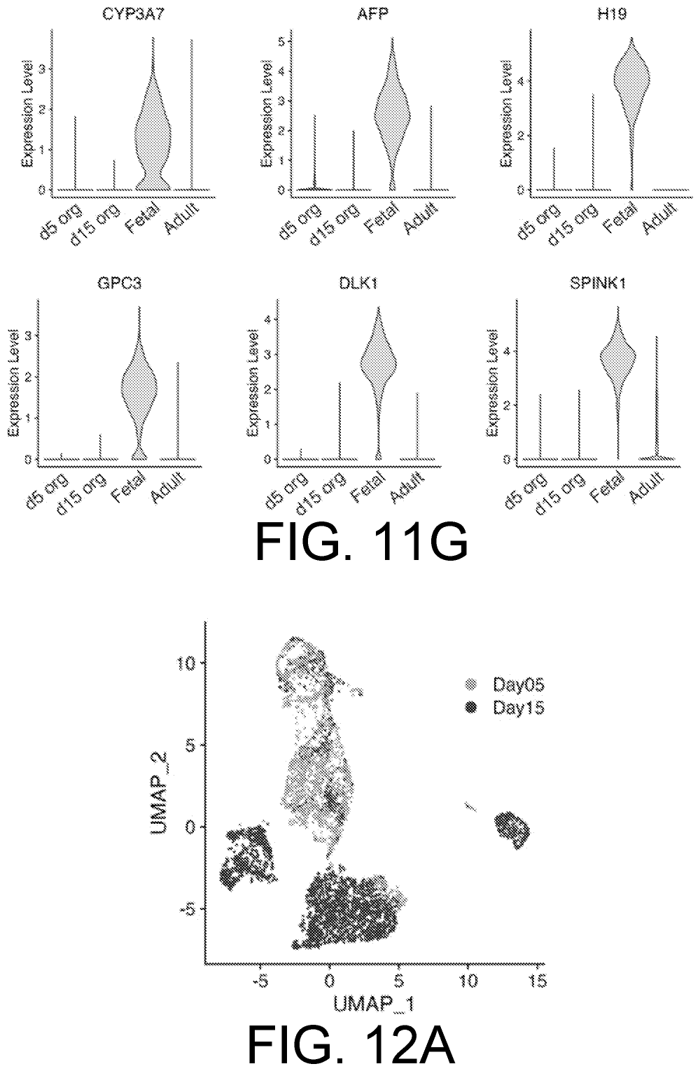

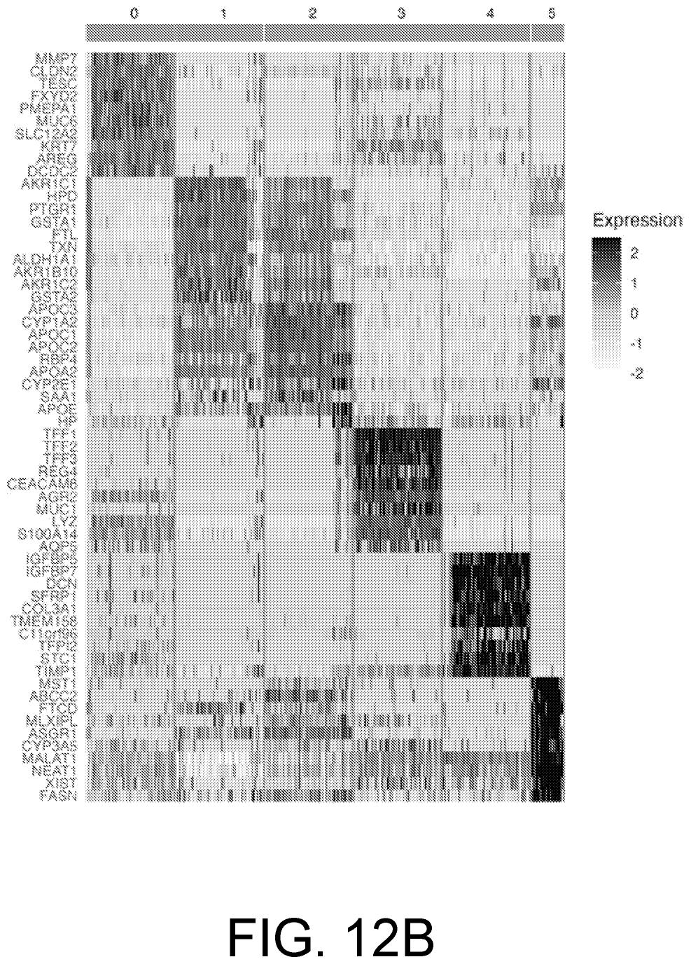

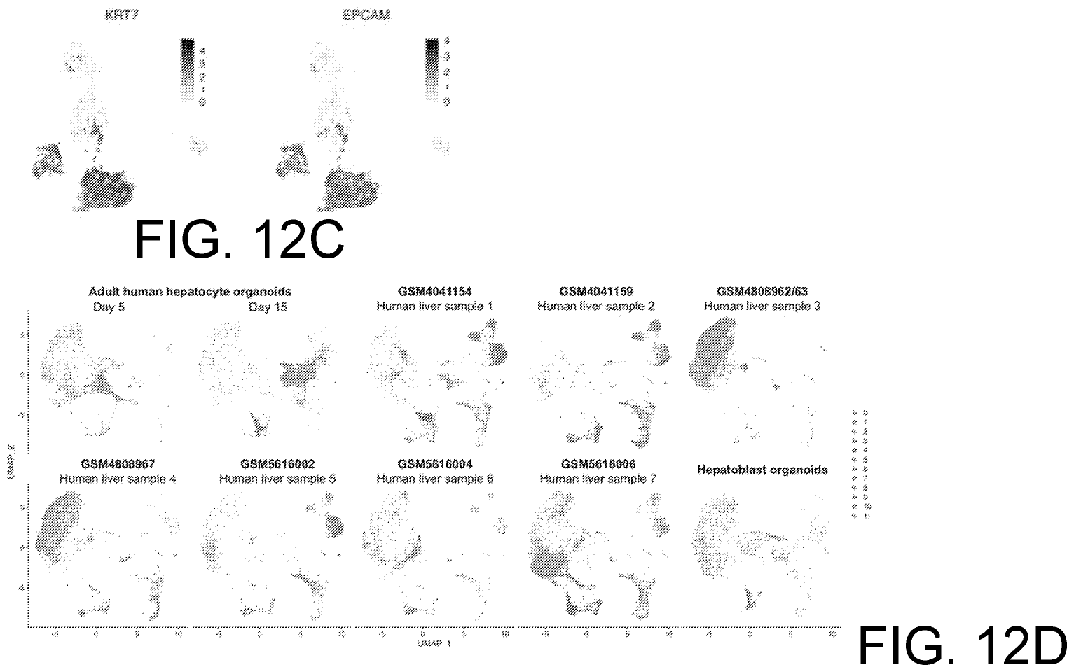

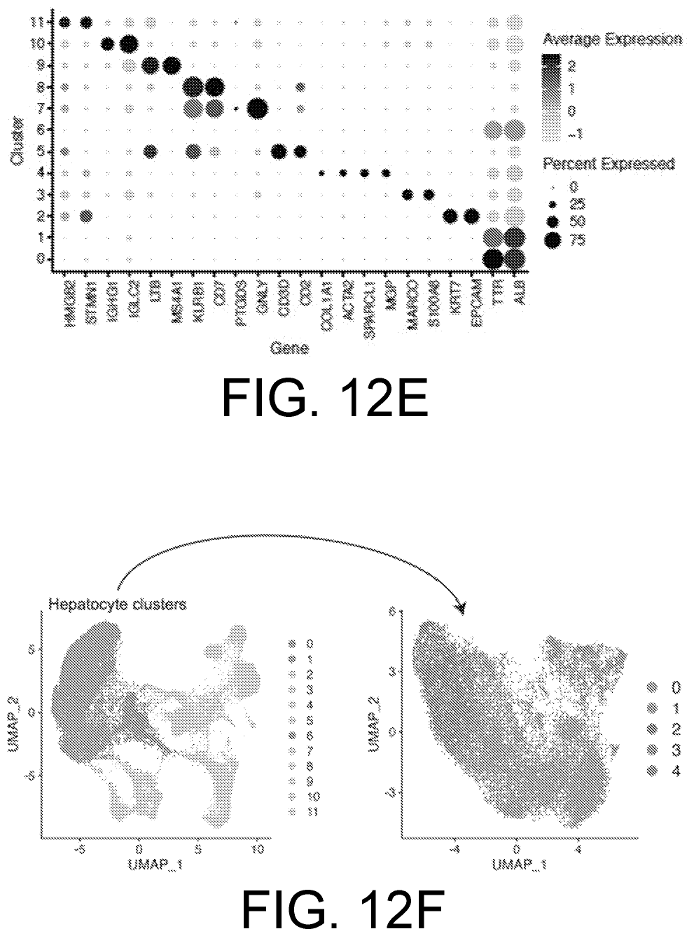

FIGS. 11A to 11G illustrate results of single-cell RNA sequencing demonstrating transcriptomic maturity and functionality of adult human hepatocytes grown as organoids in accordance with this Example. FIG. 11A shows UMAP clustering of single cells from adult human hepatocyte organoids at day 5 and day 15 in culture. Green, pink, and yellow clusters represent hepatocytes. FIG. 11B shows a UMAP of adult human hepatocyte organoids on day 5 (light orange) and day 15 (dark orange) clustered with fetal hepatoblast organoids (light grey) and primary adult hepatocytes (dark grey) from seven human liver samples. Hepatocytes clusters subset from initial UMAP (FIG. 12F) and re-clustered for subsequent analysis. Insets of adult human hepatocyte organoids for clearer visualization. FIGS. 11C to 11F show UMAP plots of adult human hepatocyte organoids from FIG. 11A, emphasizing expression of individual genes of interest, and violin plots from FIG. 11B showing distribution of expression compared to controls. Genes are divided by hepatic functional axis: FIG. 11C shows plasma protein synthesis, FIG. 11D shows lipid metabolism, FIG. 11E shows amino acid metabolism, and FIG. 11F shows drug metabolism. Violin plot groups color-coded as in FIG. 10B with d05 org=Day 5 adult human hepatocyte organoids, d15 org=Day 15 adult human hepatocyte organoids, Fetal=Fetal hepatoblast organoids, and Adult=Adult human liver. FIG. 11G illustrates violin plots from FIG. 11B showing the distribution of fetal hepatic gene expression in adult human hepatocyte organoids compared to controls. Groups color coded as in FIG. 11B with d05 org=Day 5 adult human hepatocyte organoids, d15 org=Day 15 adult human hepatocyte organoids, Fetal=Fetal hepatoblast organoids, and Adult=Adult human liver.

FIGS. 12A to 12F illustrate that cell RNA sequencing demonstrates cellular heterogeneity in adult human hepatocyte organoids and published human liver samples. FIG. 12A shows UMAP clustering of single cells from adult human hepatocyte organoids at day 5 (pink) and day 15 (blue) in culture. FIG. 12B is a heat map of the expression of top 10 most differentially expressed genes between clusters 0-5 in UMAP from FIGS. 11A and 12A. FIG. 12C shows UMAP plots adult human hepatocyte organoids emphasizing expression of KRT7 (left) and EPCAM (right). FIG. 12D illustrates UMAP of adult human hepatocyte organoids at day 5 and day 15 clustered with fetal hepatoblast organoids and primary adult hepatocytes from 7 human liver samples. Samples were aggregated for UMAP analysis then separated for visualization. FIG. 12E shows a dot plot demonstrating average expression and percent of cells expressing genes of interest in each cluster from UMAP in FIG. 12D. Genes are selected as representative markers for different cell types of the liver, informing cluster identities in bottom right. FIG. 12F is a schematic illustrating the re-clustering of all hepatocyte clusters from UMAP in FIG. 12D into the projection used in FIG. 11B.

After analysis by principal component analysis (PCA) and clustering with uniform manifold approximation and projection (UMAP), cells were segregated into six distinct populations (FIGS. 11A and 12A). Each cluster was characterized by examining differential gene expression and identifying known cell type-specific markers. Clusters 1, 2, and 5 represented hepatocytes with high expression of hepatic markers such as ALB, ALDH1A1, APOC1, and ASGR1. Clusters 0 and 3 were composed of cholangiocytes, with low expression of the above hepatic markers and high expression of biliary markers KRT7, EPCAM, and TFF1-3. Cluster 4 was composed of a stromal population expressing COL3A1, IGFBP7, and DCN (FIGS. 12B and 12C).

Having classified the cells in the adult human hepatocyte organoids based on known hepatocyte markers, the transcriptional profiles of the organoid hepatocytes to hepatocytes from adult liver were compared using a non-biased approach. Seven published scRNAseq libraries of adult human liver (Andrews, T. S. et al., Hepatology Communications 0, 2021; Payen, V. L. et al. (2021). JHEP Reports 3; Ramachandran, P. et al. (2019). Nature 2019 575:7783 575, 512-518) were compiled and integrated. The adult human liver data was clustered with the adult human hepatocyte organoid samples as well as with a fetal hepatoblast organoid library as a comparison for maturity (Wesley et al., 2022) (FIG. 12D). Cells clustered into 11 distinct populations, which we identified as hepatocytes, cholangiocytes, stromal/endothelial cells, and immune cells based on cell-type-specific gene expression (MacParland, S. A. et al. (2018). Nature Communications 2018 9:1 9, 1-21) (FIG. 12E). Cells from the adult human organoid population clustered with adult liver hepatocytes, cholangiocytes, and stromal cells, validating that each population retained transcriptional similarity to its primary cell of origin (FIG. 12D). Hepatocytes from the adult human organoids were distributed across the hepatocyte clusters from all adult datasets, despite significant variability in the individual adult human liver libraries (FIG. 12D).

To further explore the distribution of functional gene expression specifically in the hepatocyte populations, the three hepatocyte clusters (0, 1, and 6) were subset from the complete dataset (FIG. 12F). In the resulting UMAP projection, adult human organoid hepatocytes were again found to distribute evenly with hepatocytes from adult liver datasets (FIG. 11B). Genes involved in important hepatic processes were then compared between adult human organoid hepatocytes, fetal hepatoblast organoids, and hepatocytes from the integrated adult liver datasets by visualizing the distribution of expression of genes representing various axes of hepatocyte metabolic function.

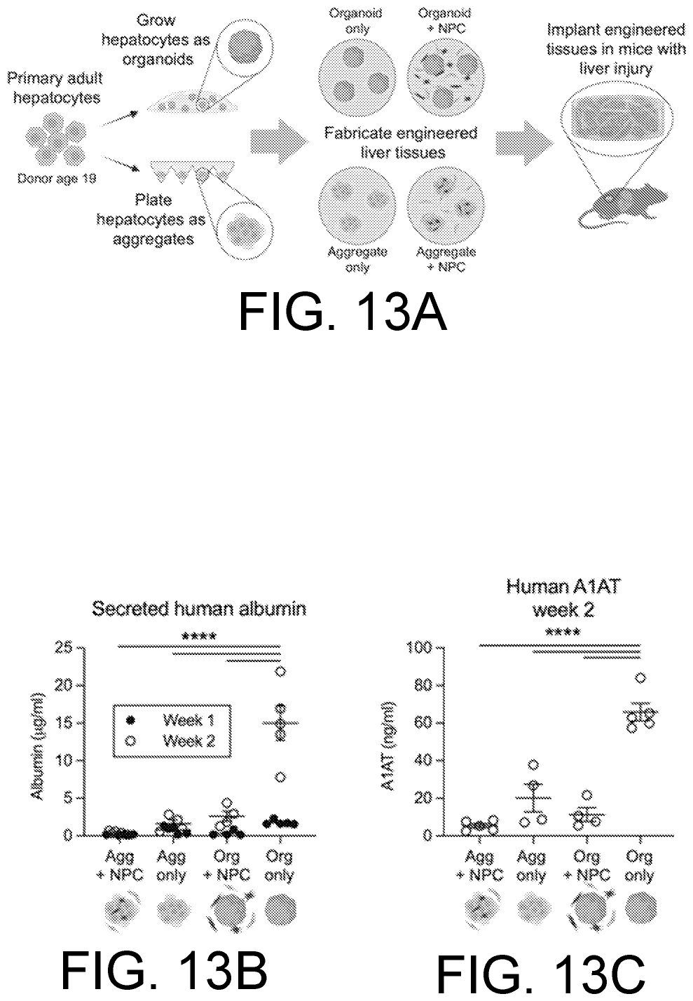

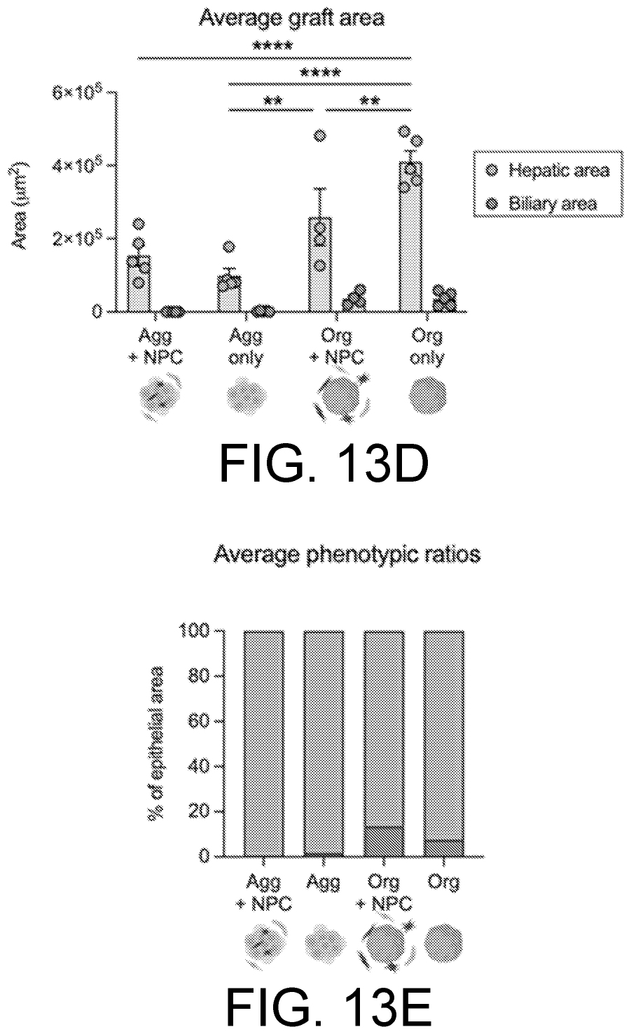

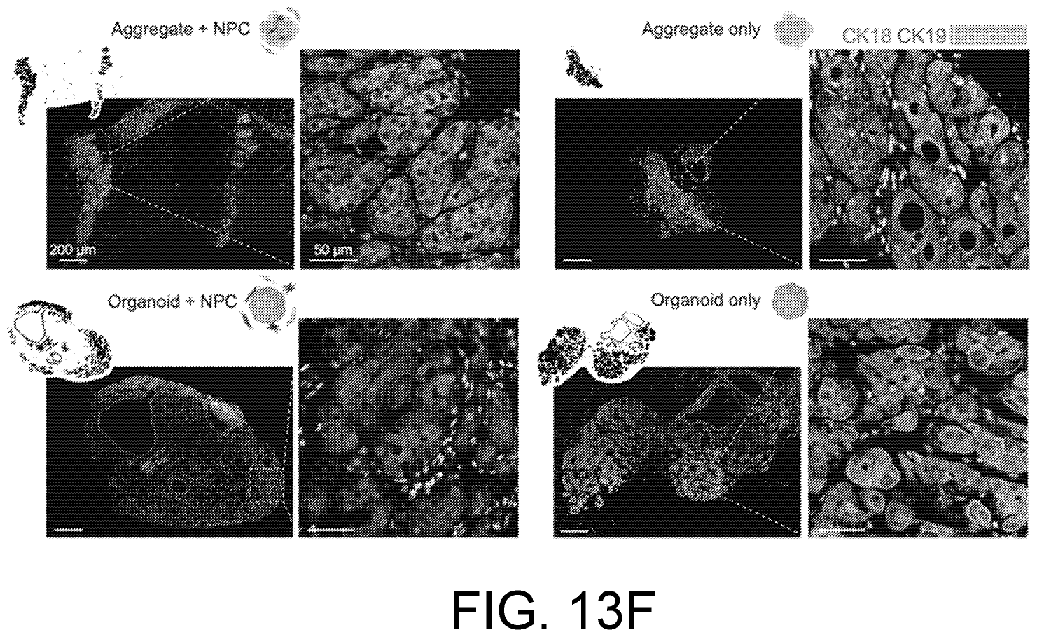

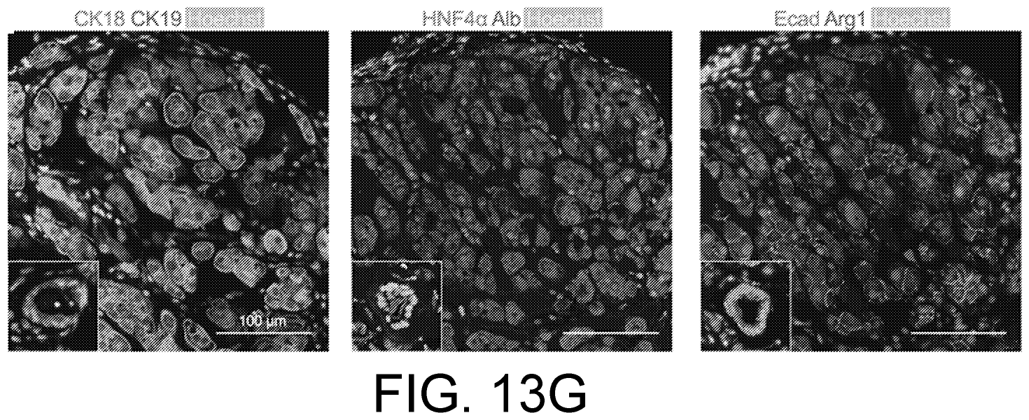

FIGS. 13A to 13G show that engineered liver tissues generated with adult human hepatocyte organoids demonstrate increased function and engraftment than those generated with hepatocyte aggregates. FIG. 13A is a schematic illustrating the in vitro culture of adult human hepatocytes as either organoids or aggregates and the subsequent fabrication and implantation of engineered liver tissues with either aggregates or organoids, with or without non-parenchymal cells. FIG. 13B shows secreted human albumin measured in mouse blood by human albumin ELISA. Data in FIG. 13B is represented as mean+/−SEM of each mouse at week 1 (black) and week 2 (white), and is analyzed using two-way ANOVA, Sidak's multiple comparisons test, ****p<0.0001. FIG. 13C indicates secreted human A1AT measured in mouse blood by human A1AT ELISA. Data in FIG. 13C is represented as mean+/−SEM of each mouse at week 2, and was analyzed using one-way ANOVA, Tukey's multiple comparisons test, ****p<0.0001. FIG. 13D illustrates the average graft area calculated from immunofluorescence images. Hepatic area was measured as CK18+/CK19− area (cyan). Biliary area was measured as CK18+/CK19+ area (magenta). Each data point represents the average graft area of all grafts found in a single mouse. Data in FIG. 13D is represented as mean+/−SEM of each mouse at week 2, and was analyzed using two-way ANOVA, Sidak's multiple comparisons test, **p<0.01, ****p<0.0001. FIG. 13E shows the average hepatic area and biliary area as a ratio of total epithelial area (CK18+) in each group. Hepatic area was measured as CK18+/CK19− area (cyan). Biliary area was measured as CK18+/CK19+ area (magenta). FIG. 13F includes representative fluorescence images used for quantification in FIGS. 13D and 13E. FIG. 13F illustrates CK18 (cyan), CK19 (magenta), and Hoechst (yellow). In FIG. 13F, scale bars=200 μm for low magnification, 50 μm for inset images. Example masks used for quantification in upper left corner of each image. FIG. 13G shows confocal images of serial sections of organoid-only graft stained for CK18 (cyan) and CK19 (magenta) (left), Alb (magenta) and HNF4c (cyan) (middle), or Arg1 (magenta) and E-cad (cyan) (right). Nuclei stained for Hoechst (yellow) in all images. In FIG. 13G, scale bars=100 μm. The inset shows serial sections of neighboring biliary area at the same scale.

Secreted plasma protein genes ALB, TTR, and RBP4 represent key hepatocyte functions and serve as early indicators of endodermal commitment toward hepatic cell fate in development (Si-Tayeb, K. et al. Developmental Cell 18, 175-189). All three genes had strong expression across the majority of cells in the hepatocyte clusters and exhibited similar gene expression to adult liver and fetal controls (FIG. 13C). Similarly, genes involved in lipid and fatty acid metabolism that are expressed in the fetal liver and throughout adult life, APOA2, APOC1, and FABP1, were strongly expressed across the entire hepatocyte population and at similar levels to adult liver and fetal controls (FIG. 13D) Segal, J. M. et al. (2019). Nature Communications 2019 10:1 10, 1-14; Wesley et al., 2022). Genes involved in glutathione and amino acid metabolism, GAMT, MGST1, and HPD, all had high expression in adult human organoid hepatocytes with similar expression to hepatocytes from adult liver. Interestingly, HPD, an enzyme involved in tyrosine metabolism, had distinctly lower expression in fetal hepatoblast organoids compared to all other conditions including adult human hepatocyte organoids, which again highlighted the mature phenotype of human hepatocyte organoids (FIG. 13E). Thus, functional genes with known expression changes over the course of hepatocyte development and maturation were more carefully queried.

As the liver develops and matures pre- and postnatally, many functional genes upregulate or downregulate to adjust to the hepatic requirements for the corresponding stage of development. Regulation of cytochrome enzymes and other genes involved in drug metabolism is especially dynamic as the liver matures. For example, gene expression of enzymes CYP2E1 and CES1 does not rise in the liver until after birth, while CYP3A7 is expressed highly in fetal liver and downregulates postnatally (Wesley et al., 2022). It was observed that expression of mature drug-metabolizing genes CYP2E1 and CES1 was high in the adult human organoid hepatocytes after several weeks of culture, similar to hepatocytes in human liver samples, while fetal controls had little to no expression (FIG. 11F). In contrast, expression of fetal drug-metabolizing enzyme CYP3A7, as well as other fetal hepatic genes AFP, H19, GPC3, DLK1, and SPINK1, was low or absent in the adult human organoid hepatocytes and control hepatocytes from adult liver (FIG. 11G). All fetal hepatic genes had high expression in fetal controls (FIG. 11G).

Taken together, these results validate that the adult human hepatocyte organoids prepared in accordance with this Example maintain differentiated and mature transcription across multiple axes of liver function. In particular, the expression of developmentally-dynamic drug-metabolizing genes emphasizes the value of adult human hepatocyte organoids for pharmaceutical applications such as drug screening.

### Adult Human Hepatocyte Organoids in Engineered Liver Tissues Rapidly Adopt Liver Morphology and Functional Maturity

When the liver is damaged beyond repair via end-stage liver disease, organ transplantation is the only curative treatment. However, an acute global shortage of donor organs leaves many patients waiting for transplants while their liver function and quality of life continue to decline (Brown, J et al., (2006). Qualitative Health Research 16, 119-136). An exciting alternative approach to overcome the scarcity of donor livers is cell-based therapy, wherein hepatic cells are engrafted orthotopically or ectopically into a patient with liver disease to assume partial responsibility for the lacking liver functions (Dwyer et al., 2021; Komori, J. et al. (2012). Nature Biotechnology 2012 30:10 30, 976-983; Stevens et al., 2017; Takebe, T. et al. (2013). Nature 499, 481-484; Wilson, E. M. et al. (2014). Stem Cell Research 13, 404-412). As liver cell-based therapies move closer to clinical translation, it will be essential to establish a reliable source of human hepatocytes. Towards this end, the value of adult human hepatocyte organoids in pre-clinical transplantation studies was established.

The utility of adult human hepatocyte organoids as a cell source within engineered liver tissues was examined, which could serve as an ectopic therapeutic option for patients suffering from a variety of forms of liver disease (Stevens et al., 2017; Takebe et al., 2013). To test the engraftment potential of hepatocytes grown as organoids within engineered tissues, adult human hepatocyte organoids were grown from a 34-year-old donor and encapsulated within fibrin hydrogel to create engineered liver tissues. These tissues were then implanted in the gonadal fat pad of FRGN (Fah−/−, Rag2−/−, Il2rg−/−, NOD) mice, a strain that experiences chronic liver injury (tyrosinemia) unless administered the treatment drug nitisinone (Wilson et al., 2014). Human albumin was then measured in mouse blood from weekly blood draws as a measure of the function of human hepatocytes within the implanted engineered tissues.

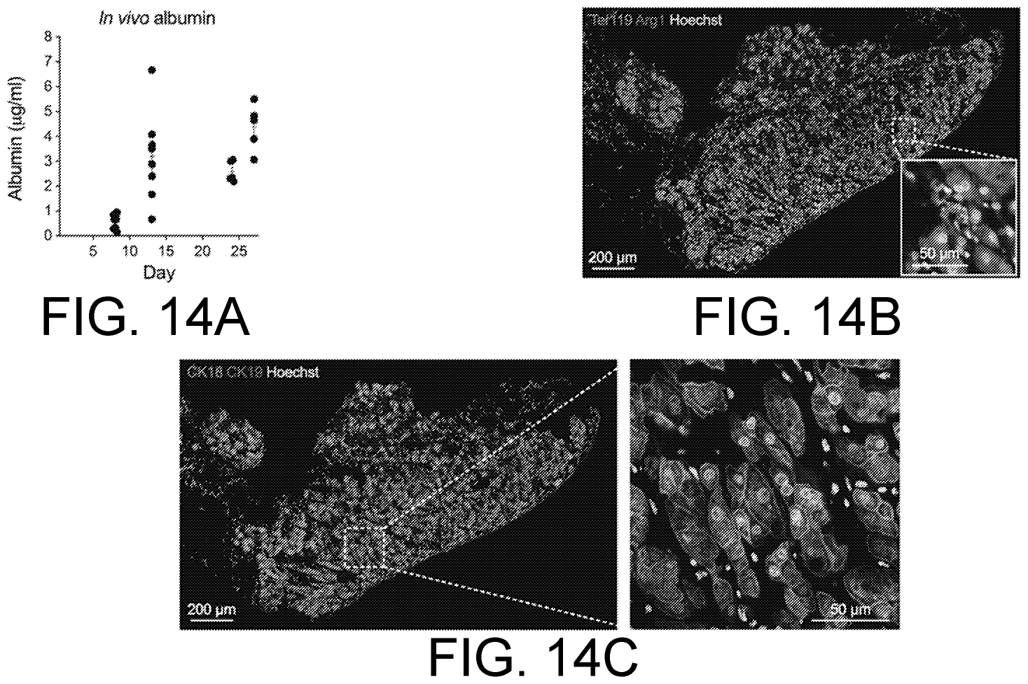

FIGS. 14A to 14C show the results of engineered liver tissues fabricated with adult human hepatocyte organoids engraft and function in vivo in a mouse model of chronic liver injury. FIG. 14A shows secreted human albumin measured in mouse blood by human albumin ELISA. Data in FIG. 14A is represented as mean+/−SEM of each mouse at different timepoints across 28 days. FIG. 14B shows an immunofluorescence image of explanted graft stained for Arg1 (magenta), Ter119 (cyan), and Hoechst (yellow). In FIG. 14B, the low magnification scale bar=200 μm, and the inset scale bar=50 μm. FIG. 14C shows an immunofluorescence image of explanted graft stained for CK18 (cyan), CK19 (magenta), and Hoechst (yellow). In FIG. 14C, the low magnification scale bar=200 μm, and the inset scale bar=50 μm.

Human albumin increased over 4 weeks in vivo, suggesting both a functional increase over time as well as a connection to host vasculature (FIG. 14A). Excised grafts displayed large areas in which most cells stained positively for antigens expressed by hepatocytes, CK18 and Arg1 (FIGS. 14B and 14C). Occasional CK19+ cells with hepatocyte morphology were also present in grafts, perhaps suggesting that some engrafted cells adopt a progenitor phenotype (Michalopoulos, G. K. and Khan, Z. (2015). Gastroenterology 149, 876-882). The mouse red blood cell marker Ter119 stained small, localized areas throughout the graft between Arg1+ hepatocytes, indicating that implanted cells were being supplied with host blood (FIG. 14B).

The substantive size of grafts in this initial study (FIGS. 14A to 14C) was surprising, given that no other cell populations beyond the adult human hepatocyte organoids themselves were included within the engineered tissues. Previous studies had shown that optimal engraftment of engineered tissues built from other hepatocyte sources (e.g., primary human hepatocyte aggregates) (Stevens, K. R. et al. (2013). Nature Communications 4, 1847; Stevens et al., 2017) and iPS-derived hepatocytes (Takebe et al., 2013) required the addition of endothelial and stromal cells (e.g., fibroblasts, mesenchymal stromal cells) prior to tissue transplantation. The ability to remove these cell populations from engineered tissues would greatly simplify the clinical development and approval pipeline by creating a simpler product and would also reduce safety concerns of tumorigenicity and stromal cell overgrowth (Chen, A. X. et al., Advanced Functional Materials 30, 1910442; Stevens et al., 2013, 2017).

Next, engraftment efficiency of engineered tissues derived using two different primary adult human hepatocyte culture methods—adult human hepatocyte organoids developed here, or “aggregates” developed previously (Stevens et al., 2017)—both in the presence and absence of non-parenchymal cells (NPC) were directly compared.

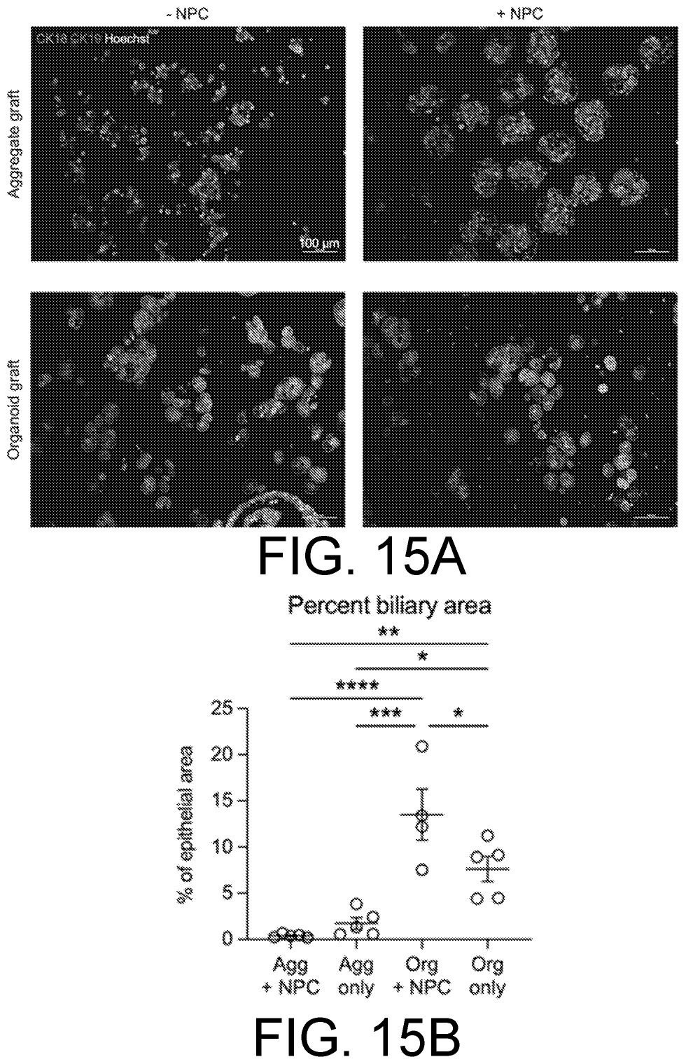

FIGS. 15A and 15B shows the results of adult human hepatocyte organoids and aggregates with and without non-parenchymal cells before implantation in FRGN mice. FIG. 15A shows representative fluorescence images of each group (aggregates or organoids+/−NPC) at implant stained for CK18 (cyan), CK19 (magenta), and Hoechst (yellow). In FIG. 15A, scale bars=100 μm. FIG. 15B shows percent biliary area calculated from immunofluorescence images as CK18/CK19+ area/CK18+ area. Each data point represents the average percent biliary area of all grafts found in a single mouse. Data in FIG. 15B is represented as mean+/−SEM of each mouse at week 2. One-way ANOVA, Tukey's multiple comparisons test, *p<0.05, **p<0.01, ***p<0.001, ****p<0.0001.

Engineered tissues that contained either adult human hepatocyte organoids or aggregates with or without fibroblasts and endothelial cells, collectively referred to as non-parenchymal cells (NPCs) (FIG. 13A) were built. Both organoids and aggregates were generated from the same 19-year-old human hepatocyte donor. Notably, this was a different donor than was used in the previous in vivo experiment (FIGS. 14A to 14C), thus together these studies enabled assessment of engraftment of two different adult donors. Again, blood was drawn from implanted mice to assess human liver protein (albumin) production as a metric for hepatic functionality of implants. After one week of engraftment, the group of animals receiving engineered tissues with only adult human hepatocyte organoids had on average >2-fold more human albumin than the organoid+NPC group or either aggregate group (FIG. 13B). By week two, the differences between groups were even more pronounced with the organoid-only group producing ˜6× more albumin than the organoid+NPC group, as well as significantly more than both aggregate+/−NPC control groups, at up to ˜25× more albumin (FIG. 13B). Similarly, human alpha-1 antitrypsin (A1AT) was also identified in the mouse blood of the organoid-only group at ˜6× more than the organoid+NPC, and greater than both aggregate+/−NPC groups (up to ˜12× difference; FIG. 13C).

To further explore the engraftment efficiency of human cells within tissue grafts from each group, implanted tissues were excised and immunostained. Human CK18, a marker of both hepatocytes and cholangiocytes, was identified in grafted areas in all mice (FIGS. 13D to 13F). Using CK19 as a marker to differentiate between hepatocyte and biliary areas, graft size and phenotype were quantified in all groups. Importantly, and in corroboration with secreted protein data (FIGS. 13B and 13C), hepatic graft area (CK18+/CK19−) was found to be significantly larger in organoid-only grafts compared to all other groups (FIG. 13D). Significant biliary area (CK18+/CK19+) was also found in both organoid groups, comprising 13.5% of organoid+NPC grafts and 7.6% percent of organoid-only grafts (FIGS. 13D, 13E, and 15B).

While the presence of secreted human proteins albumin and A1AT in mouse blood suggested that the CK18+/CK19− cells, which constituted >86% of the human cell area in organoid grafts, were indeed human hepatocytes in engrafted tissues, we sought to further establish their hepatocyte identity via immunostaining (FIGS. 13F and 13G). It was found that these cells also expressed hepatocyte functional proteins Alb and Arg-1, transcription factor HNF4α, and epithelial cell-cell junction protein E-cad, and were generally negative for CK19, all consistent with a human hepatocyte phenotype (FIG. 13G). Furthermore, cells that stained positively for CK18, Alb, Arg1, HNF4α, and E-cad also exhibited hepatocyte morphology with a low nuclear-to-cytoplasmic ratio, occasional binucleation, and tightly packed cells that collectively self-assembled to form elongated structures reminiscent of hepatocyte cords in the mature liver (FIG. 13G).

Conversely, CK19+ cells were negative for hepatocyte markers but positive for E-cad (FIG. 13G, insets) and had small, densely packed cuboidal cell morphologies, all consistent with cholangiocytes. In explanted tissue grafts, these cells were generally found in self-assembled tubular structures resembling bile ducts, with diameters ranging from ˜15 μm-800 μm. Notably, the percent of biliary area in organoid-only grafts was significantly lower than in the organoid+NPC group (FIGS. 13E and 15B), suggesting that non-parenchymal cells may play a role in regulating the ratio of human hepatocyte versus cholangiocyte populations in engrafted tissues.

Taken together, these findings demonstrate that “priming” adult human hepatocytes in vitro as organoids provides a highly functional cell source in engineered liver tissues that robustly engrafts and spatially reorganizes to create structures that morphologically, phenotypically, and functionally resemble both human hepatocyte and cholangiocytes populations and that exogenous non-parenchymal cell populations are not needed to facilitate tissue engraftment or assembly.

### Discussion

This Example provides a novel adult human hepatocyte organoid culture method that closely maintains the phenotype, morphology, transcription, and functional characteristics of hepatocytes in human liver. These adult human hepatocyte organoids maintain numerous axes of mature human liver function including protein production, nitrogen metabolism, drug metabolism, and cholesterol and glycogen storage. Notably, through both gene expression analysis and direct measurement of functional activity, maintenance of drug-metabolizing enzymes was identified, which confirms the maturity of adult human hepatocyte organoids and represent an axis of adult liver function. Furthermore, engineered liver tissues containing adult human hepatocyte organoids generate substantively larger and more functional hepatic grafts after implantation compared to those created using prior methods (Stevens et al., 2017).

These findings, demonstrating that organoid culture primes hepatocytes for different engraftment outcomes, have powerful translational implications, as enhancing hepatic engraftment efficiency would concomitantly reduce the cell sourcing production burden. One potential mechanistic reason that organoid culture may prime hepatocytes for enhanced engraftment is that organoid culture recovers hepatocytes that have been damaged by cryopreservation, in which ice crystal formation and anoikis (Smets et al., 2002; Stéphenne, Najimi and Sokal, 2010; Yoshida et al., 2020) and downregulation of important adhesion molecules such as E-cadherin (Stéphenne et al., 2010; Terry et al., 2007) lead to poor cryo-recovery and plating efficiency after thaw. Adult human hepatocyte organoids exhibit strong E-cadherin expression and reliable organoid formation across multiple human donors, suggesting that organoid culture may help to recover hepatocytes from cryo-induced injury and downregulation of adhesion molecules. Additionally, organoid culture may serve as a purification step to enrich healthy, expandable hepatocytes and dilute those cells that could not recover from cryopreservation, improving the ratio of healthy:damaged hepatocytes in engineered liver tissues. Those enriched, healthy hepatocytes also demonstrate an improvement in transcriptional maturity throughout the culture timeline, which has been suggested to improve cell transplantability and engraftment in engineered liver tissues (Billerbeck et al., 2016; Haridass et al., 2009). Thus, organoid culture may prime hepatocytes for implant by purifying healthy cells and/or providing an environment in which hepatocytes can recover from cryo-damage and can re-establish maturity, homeostatic cell-cell adhesions, morphology, and function.

In addition to demonstrating transcriptional maturity, the single-cell RNA sequencing data described with reference to this Example highlights that drug metabolism genes shift expression over time in organoids. Downregulation of CYP enzymes during liver regeneration in rodents is a well-documented phenomenon as cells undergo significant growth (Habib, S. L. et al. (1994). Toxicology and Applied Pharmacology 124, 139-148; Marie, I. J. et al. (1988). Biochemical Pharmacology 37, 3515-3521; Trautwein, C. et al. (1997). J Hepatol 26, 48-54). Thus, adult human hepatocyte organoids may be similarly shifting metabolism towards cell growth and away from xenobiotic metabolism in the early days of culture, then returning to normal adult liver transcription and function as hepatocytes reach homeostasis. Additionally, low expression of fetal genes in adult human hepatocyte organoids confirms their maturity but also validates that while growing they do not adopt a malignant transcriptional profile, as fetal genes such as AFP, H19, and GPC3 are often upregulated when adult hepatic cells become cancerous (Si-Tayeb et al., 2010; Spear, B. T. et al. (2006). Cellular and Molecular Life Sciences CMLS 2006 63:24 63, 2922-2938).

Another impactful and surprising finding in this work was the demonstration that adult human hepatocyte organoids exhibit significant functional improvement when implanted without non-parenchymal cell support. Historically, implanted hepatic cells—either primary human hepatocytes or iPSC-derived hepatic endoderm—have demonstrated decreased function in the absence of non-parenchymal cells. The results described herein demonstrate afunctional, phenotypic, and translational advantage of adult human hepatocyte organoids, as exclusion of superfluous cell types may save time and cost in engineered tissue production, as well as speed regulatory approval of implantable tissues.

Interestingly, it was further discovered that the addition of fibroblasts and endothelial cells increased the ratio of biliary to hepatic cells within engrafted engineered tissues composed of adult human hepatocyte organoids. This finding is particularly notable when considered together with previous findings demonstrating that paracrine signals between mouse ductal cell organoids and nearby mouse portal fibroblasts induce biliary proliferation (Cordero-Espinoza, L. et al. (2021). Cell Stem Cell 28, 1907-1921.e8). Together, these findings suggest that exogenous fibroblasts in engineered liver tissues may stimulate proliferation of residual cholangiocytes within implanted adult human hepatocyte organoids, thus expanding the proportion of cholangiocytes in the resultant graft (Cordero-Espinoza et al., 2021). This has major clinical ramifications, as such cellular interactions could be leveraged to finely tune the identities, proportions, and morphogenesis of several liver cell populations within engineered tissues, similar to multicellular assembly processes in human liver development and regeneration (Gordillo, M. et al. (2015). Development 142, 2094-2108; Michalopoulos, G. K. (2007) Journal of Cellular Physiology 213, 286-300; Si-Tayeb et al., 2010).

As liver cell therapy moves closer to patients, further improvements such as generating adult human hepatocyte organoids at therapeutically relevant, human-organ scales, will accelerate translation. The adult human hepatocyte organoids described here could broadly enable basic and clinical studies in areas such as therapeutic regenerative medicine, pharmaceutical screening, and generating patient-specific organoids for precision medicine.

### Experimental Model and Subject Details

### Mice

All surgical procedures were conducted according to the protocol approved by the University of Washington Institutional Animal Care and Use Committee. Mice for all implant surgeries were 12- to 20-week-old Fah−/−, Rag2−/−, Il2rg−/− on the NOD strain background (FRGN) from Yecuris.

### Cell Lines

HA-R-spondin1-Fc 293T cells (Cultrex, R&D 3710-001-01) and L-Wnt3a cells (ATCC CRL-2647) were used to generate conditioned medium of R-spondin1 and Wnt3a (described below). Human umbilical vein endothelial cells (HUVEC, Lonza C2519A) were grown in EGM-2 medium (Lonza) and neonatal normal human dermal fibroblasts (NHDF, Lonza CC-2509) were grown in DMEM+10% FBS+1% penicillin-streptomycin. HUVECs and NHDFs were used in engineered liver tissues between passages 4-8.

### Primary Cells

Cryopreserved primary human hepatocytes were purchased through Thermo Fisher (Cat #HMCPIS (lots Hu8366, Hu8373, Hu8300, Hu8287, and Hu1880) HMCPMS (lot Hu8360), and HMCPTS (lots Hu8339 and Hu8375)) and cultured as organoids, as described below.

### Methods

### Conditioned Medium

Rspo1 conditioned medium was generated according to manufacturer's instructions. Briefly, Rspo1 cells were grown in dishes until confluent, given 50 ml of conditioning medium (DMEM/F12 and 1% GlutaMAX) and incubated at 37° C. for 7-10 days. Wnt3a conditioned medium was generated by growing Wnt3a cells to confluence, replacing their medium with conditioning medium (DMEM+GlutaMAX, 10% FBS, 1% penicillin-streptomycin, 1% non-essential amino acids, 1% sodium pyruvate, and 1% HEPES) and incubating at 37° C. for 4 days. Conditioned medium was tested using the Super TopFlash assay with HEK 293 STF cells (ATCC CRL-3249), according to manufacturer's instructions.

### Organoid Culture of Hepatocytes

Cryopreserved hepatocytes were thawed into 37° C. medium and quickly spun down at 70×g. Cells were mixed with 45% organoid medium and 55% Matrigel and seeded in 20 μl droplets in each well of 48-well plates. After Matrigel had solidified, 200 μl of organoid medium was added to each well. Organoid medium: 40% Basal medium (DMEM/F12, 1× GlutaMAX, 1×HEPES, 1× penicillin-streptomycin, 2% B27, 10 mM nicotinamide, 1.25 mM N-acetyl cysteine, 10 nM gastrin), 50% Wnt3a conditioned medium, 10% Rspo1 conditioned medium, 50 ng/ml EGF, 5 μM A83-01, and 10 μM y-27632. Medium was refreshed every 2-3 days with care not to disturb the Matrigel droplet. Organoid size and growth were quantified from minimum intensity projections of brightfield z-stacks taken on a Nikon Eclipse Ti inverted high-resolution widefield microscope.

### Brightfield Microscopy and Morphometric Analysis

Brightfield z-stack images were taken in the center of each organoid well at multiple timepoints on a Nikon Eclipse Ti inverted high-resolution widefield microscope. Z-stacks were flattened into a single image using a minimum intensity projection (MinIP) with FIJI or Nikon NIS-Elements software. Organoid size and growth were quantified from MinIP images in ImageJ software and graphed with GraphPad Prism software.

### Organoid Histology, Immunostaining, and Microscopy

Organoids were harvested from Matrigel and fixed in 4% PFA at room temperature for 30 minutes, then embedded in HistoGel (VWR). For 2D histology, HistoGel pellets were dehydrated in ethanol, embedded in paraffin, and sectioned using a microtome (5-6 mm sections). Immunofluorescence staining of organoids was performed by deparaffinizing sections, performing antigen retrieval, blocking with normal donkey serum for 1 hour at room temperature, then incubating with primary antibody overnight at 4° C. The following day, sections were washed with PBS-T and then incubated with secondary antibody+Hoechst 33342 (Thermo H3570) for 1 hour at room temperature, washed, and mounted with Fluoromount-G (Invitrogen). For 3D imaging, organoids in HistoGel were blocked in bovine serum albumin and normal donkey serum overnight at room temperature then incubated with primary antibodies for 24 hours at 37° C. After washing for 6 hours at room temperature, organoids were incubated with secondary antibodies overnight at 37° C. Finally, organoids were washed for 6 hours at room temperature and then cleared by bathing in Ce3D solution (Li, W. et al. (2017). Proc Natl Acad Sci USA 114, E7321-E7330) overnight at room temperature with Hoechst 33342. All antibody information is included in Table 2. Images were obtained using a Nikon Eclipse Ti inverted high-resolution widefield microscope, a Nikon A1R scanning confocal microscope, or a Leica SP8 confocal microscope. Images were processed using Adobe Photoshop or ImageJ software. For morphometric analyses, images were thresholded and pixels were measured using ImageJ software.

TABLE 2
Antibody information

| Antibody | Vendor/Product # | Species | Dilution |
|---|---|---|---|
| Human cytokeratin-18 | Dako M701029-2 | Mouse | 1:25 |
| Cytokeratin-19 | Abcam ab52625 | Rabbit | 1:100 |
| Human albumin | Bethyl A80-129A | Goat | 1:100 |
| Arginase-1 | Sigma HPA003595 | Rabbit | 1:200 |
| MRP2 | Thermo TA812520 | Mouse | 1:500 |
| HNF4a | Abcam ab201460 | Rabbit | 1:100 |
| Ter119 | BD Pharmingen 550565 | Rat | 1:100 |
| Ecad | R&D AF748 | Goat | 1:100 |
| Ki67 | Abcam ab16667 | Rabbit | 1:500 |
| Goat anti-mouse 555 | Invitrogen A21127 | Goat | 1:500 |
| Donkey anti-mouse 555 | Invitrogen A31570 | Donkey | 1:1000 |
| Donkey anti-rabbit 594 | Invitrogen A21207 | Donkey | 1:1000 |
| Donkey anti-rabbit 647 | Invitrogen A31573 | Donkey | 1:1000 |
| Donkey anti-goat 488 | Invitrogen A11055 | Donkey | 1:1000 |
| Donkey anti-goat 647 | Invitrogen A21447 | Donkey | 1:1000 |
| Donkey anti-rat 488 | Invitrogen A21208 | Donkey | 1:1000 |

### Functional Analyses

Human albumin and alpha-1-antitrypsin within organoid medium or mouse blood were measured with ELISA (Bethyl E80-129 and E88-122). Urea was measured in organoid medium using a BUN assay according to manufacturer's instructions (Fisher SB0580250). CYP3A4 induction was assessed by adding CYP inducers (25 μM rifampin or 25 μM dexamethasone) or vehicle controls (DMSO or EtOH) to organoid cultures for 3 days after which CYP3A4 activity was measured on organoid cultures using the P450-Glo CYP3A4 Assay (Promega V9001). Variability in cell numbers was normalized using the CellTiter-Glo assay (Promega G9681). Bile canalicular transport was assessed by incubating organoids for 30 minutes with 2 μg/ml 5-[and-6]-car-boxy-2′,7′-dichlorofluorescein diacetate (CDFDA, Sigma 21884), a fluorescent MRP2 substrate. After incubation organoids were imaged on a Nikon Eclipse Ti inverted microscope with a Yokogawa W1 spinning disk head. Low-density lipoprotein (LDL) uptake was assessed by incubating organoids with 20 μg/ml Dil-LDL, a fluorescently labelled LDL (Thermo L3482), for 3 hours at 37° C. with Hoechst 33342 diluted 1:1000. After incubation organoids were imaged on a Leica SP8 confocal microscope. Finally, glycogen accumulation was assessed with periodic acid-Schiff staining (PAS, Sigma, 395B-1KT) according to manufacturer's instructions. Functional analyses were performed between days 14-19 in culture.

RNA Isolation and qRT-PCR

Organoids were lysed in Qiazol (QIAGEN) or Trizol (Thermo) and RNA was extracted using the phenol-chloroform extraction method. RNA was reverse transcribed into cDNA using random hexamer primers and a Superscript III first-strand synthesis kit (Thermo 18080093) according to manufacturer's instructions. qPCR was performed using SYBR-green (BioRad 1725122). The 2^{dCT} method (Livak, K. J. and Schmittgen, T. D. (2001). Methods 25, 402-408) was used to report fold change in gene expression compared to internal control gene GAPDH. Primer sequences are included in Table 3.

TABLE 3
Primer sequences

| Gene | Forward primer | Reverse primer |
|---|---|---|
| SERPINA1 | GATCAACGATTACGTGG | CCTAAACGCTTCATCAT |
| | AGAAGG | AGGCA |
| | (SEQ ID NO: 1) | (SEQ ID NO: 2) |
| AFP | AGTGAGGACAAACTATT | ACACCAGGGTTTACTGG |
| | GGCCT | AGTC |
| | (SEQ ID NO: 3) | (SEQ ID NO: 4) |
| ALB | GAGACCAGAGGTTGATG | AGTTCCGGGGCATAAAA |
| | TGATG | GTAAG |
| | (SEQ ID NO: 5) | (SEQ ID NO: 6) |
| ARG1 | GCCAAGTCCAGAACCAT | AGCAGACCAGCCTTTCT |
| | AGG | CAA |
| | (SEQ ID NO: 7) | (SEQ ID NO: 8) |
| KRT19 | TCCGAACCAAGTTTGAG | CCCTCAGCGTACTGATT |
| | ACG | TCCT |
| | (SEQ ID NO: 9) | (SEQ ID NO: 10) |
| CYP3A4 | CAGCCTGGTGCTCCTCT | ACCATCATAAAAGCCCC |
| | ATC | ACA |
| | (SEQ ID NO: 11) | (SEQ ID NO: 12) |
| CYP1A2 | ACCTTGTGACCAAGCCT | AAGGAGGAGTGTCGGAA |
| | GAG | GGT |
| | (SEQ ID NO: 13) | (SEQ ID NO: 14) |
| TF | TGATTGCATCAGGGCCA | GCCAGGTAAGCATCATA |
| | TTG | CACCA |
| | (SEQ ID NO: 15) | (SEQ ID NO: 16) |

### Organoid Digestions and Barcoding

Organoids were grown in Matrigel under mature human hepatocyte culture conditions for 5 or 15 days, as described above. At each timepoint, organoids were pooled and suspended in TrypLE (Thermo) for 20-30 minutes until they had digested to single cells. The single-cell suspension was mixed with 1% BSA to prevent clumping and sticking to tubes/tips. Immediately before partitioning, cells were filtered through a FlowMi Cell Strainer (Belart) to remove cell clumps and debris. Cells were counted then loaded into a 10× Chromium flow cell using the Single Cell 3′ v3 kit. 10× genomics barcoding and library construction were performed according to manufacturer's recommendations and generated libraries from 2327 cells at day 5 and 3411 cells at day 15 in culture.

### Single-Cell RNA Sequencing and Analysis

Libraries were sequenced using an Illumina NextSeq 2000. Reads were aligned to the human genome GRCh38-2020-A and counted with the 10× Cell Ranger pipeline version 6.1.1 (Zheng, G. X. Y. et al. (2017). Nature Communications 2017 8:1 8, 1-12). The day 5 organoid library generated 74,577 mean reads per cell with 1,320 median genes per cell. The day 15 organoid library generated 41,264 mean reads per cell with 2,296 median genes per cell. Cell Ranger outputs were loaded into R using the Seurat v4.0 package (Stuart, T. et al. (2019). Cell 177, 1888-1902.e21) and all bioinformatic analysis was performed following the recommended vignettes for data integration and clustering as described by the Satija lab. First, cell filtering was performed on individual libraries to remove cells with >500 genes and >25% mitochondrial RNA content to exclude fragments and dying cells from analysis; 1849 and 2796 cells remained from days 5 and 15, respectively. Next, global-scaling normalization and highly variable feature selection were performed. Cells were assigned a cell cycle score which was regressed during data scaling to lessen the influence of these genes from clustering and prevent cell cycle genes from masking variations in liver gene signatures between cells. Two integrated datasets were generated: an integrated day 5 and day 15 organoid sample, and an integrated sample of day 5 and day 15 organoids, a publicly available hepatoblast sample (ArrayExpress under accession E-MTAB-7189), and multiple human liver datasets (GEO under accession numbers GSM4808962 and GSM4808963, GSM4808967, GSM5615002, GSM5616004, GSM5616006, GSM4041154, and GSM4041159). Integrated datasets were scaled, principal component analysis was performed, followed by UMAP clustering, and gene expression was visualized to assign cluster identities to known liver cell types. All plots were generated using R Studio and the Seurat package.

### Cell Aggregation

To generate hepatocyte aggregates, primary human hepatocytes were thawed, counted, and plated into pyramidal microwells as described previously (Stevens et al., 2013, 2017) at a concentration of 100 hepatocytes per microwell. If cells were aggregated with fibroblasts, NHDFs were trypsinized, counted, and added to pyramidal microwells at a concentration of 160 NHDFs per microwell immediately before adding hepatocytes. Cells were incubated at 37° C. overnight to form aggregates.

### Fabrication of Engineered Liver Seeds

Hepatocyte organoids were digested out of Matrigel (Corning) with Cell Recovery Solution (Corning), washed once in PBS, and resuspended in diluted fibrinogen. If HUVECs or NHDFs were included in engineered liver tissues, they were suspended in diluted thrombin. Diluted thrombin with or without non-parenchymal cells was mixed with an equal volume of hepatocyte organoids in diluted fibrinogen and 60 ul was pipetted into 6 mm PDMS gaskets. After 30 minutes of incubation, fibrin/cell gels were released from gaskets and implanted in mice as engineered liver tissues. Final cell concentrations were 1.25 million HUVECs/ml, 3.6 million NHDFs/ml, or 2.25 million hepatocytes/ml. Fibrin was used at a final concentration of 10 mg/ml.

### Implantation and Induction of Liver Injury

12- to 20-week-old FRGN mice (Fah−/−, Rag1−/−, Il2rg−/−, on a NOD background) were anesthetized with isofluorane. Three organoid tissues were sutured onto the gonadal fat pads of each mouse. Immediately after surgery, nitisinone (NTBC) was withdrawn from animals' drinking water for 14 days to induce liver injury. For experiments that extended beyond 14 days, NTBC was then reintroduced to the drinking water to allow for mouse recovery, then removed again after 4 days for the remainder of the experiment.

### Tissue Harvesting and Histology

Organoid tissues were identified using the surgical suture as a landmark then were excised and fixed in 4% paraformaldehyde for 48 hours at 4° C. Excess fat was removed from around the implant. Trimmed grafts were then dehydrated, paraffin-embedded, and sectioned on a microtome. Sections were then immunostained with antibodies listed in Table 2 according to the methods in “Organoid histology, immunostaining and microscopy.”

### Quantification and Statistical Analysis

### Morphometry

Collected immunofluorescence images were analyzed using ImageJ software. Images were thresholded and masks were generated for each channel. Epithelial area was measured from CK18+ masks. Hepatic area was measured from CK18+ masks-CK19+ masks. Biliary area was measured from CK18+/CK19+ masks.

### Statistical Analysis

Data in graphs are expressed as the mean±SEM, as denoted in figure legends. Statistical significance was determined with PRISM software using t-test, one-way ANOVA, or two-way ANOVA followed by Dunnett's, Sidak's, or Tukey's multiple comparison test, as denoted in figure legends.

### Second Experimental Example

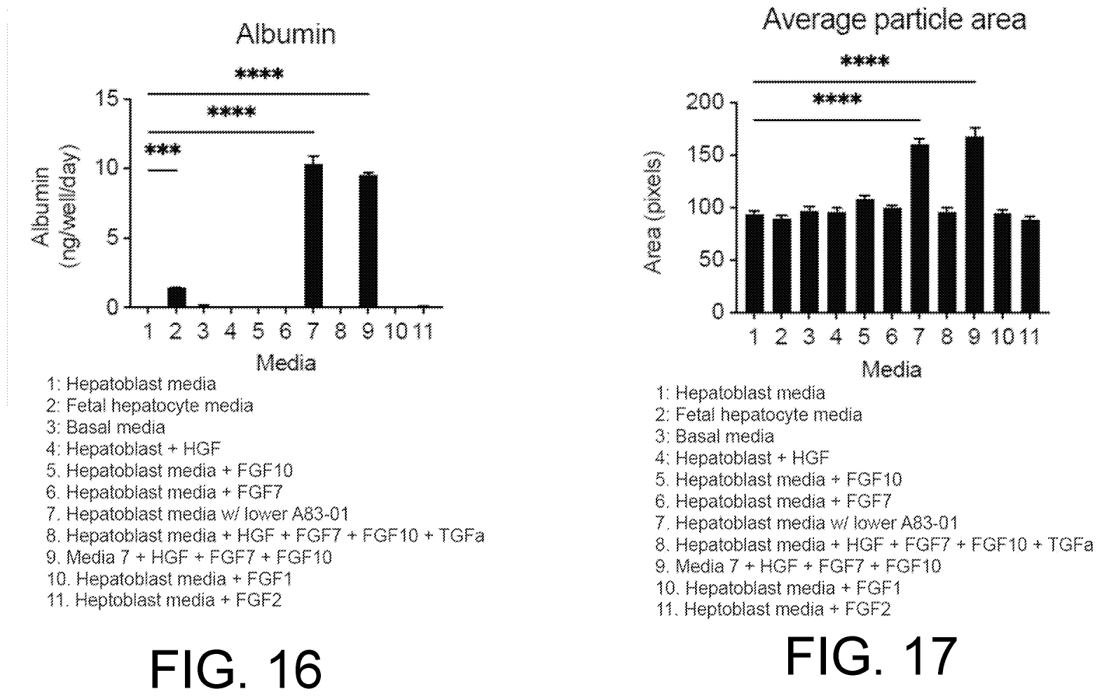

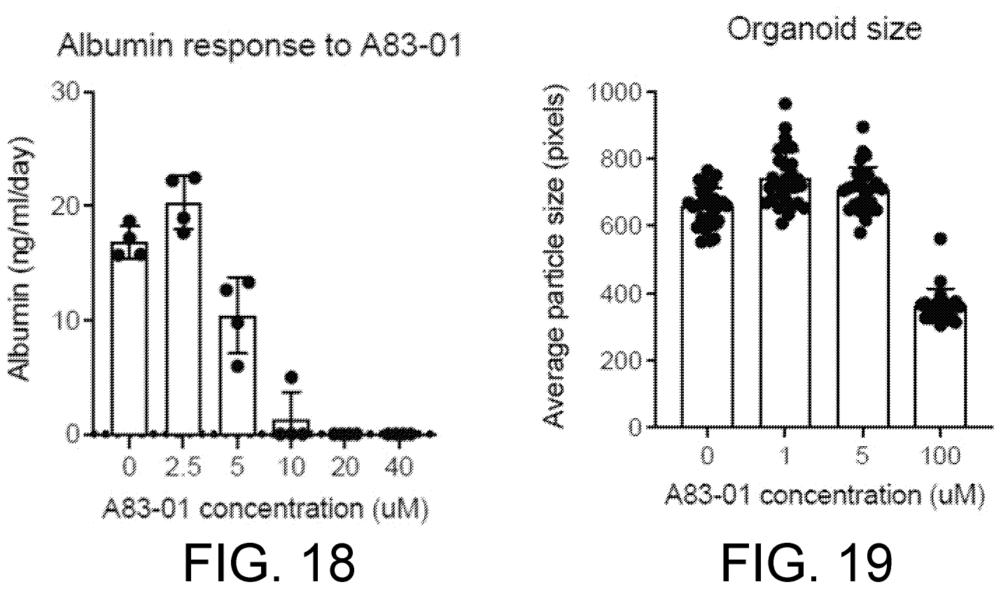

In this Example, adult human hepatocytes were grown in different concentrations of A83-01, ranging from 0 nM to 40 nM. FIG. 16 illustrates albumin secretion of hepatocytes grown with 6 different concentrations of A83-01 measured by human albumin ELISA. FIG. 17 illustrates average cross-sectional area of organoids in individual wells grown in 4 different concentrations of A83-01 ranging from 0 to 100 nM. Data is represented as the mean, plus or minus standard deviation of more than 20 wells. FIG. 18 illustrates albumin secretion measured by human Albumin ELISA, comparing adult human hepatocyte organoids grown in 5 different concentrations of A83-01 ranging from 0 nM to 40 nM. The results of these studies illustrate that medium containing less than 50 nM of A83-01 is effective for adult hepatocyte growth.

### Third Experimental Example

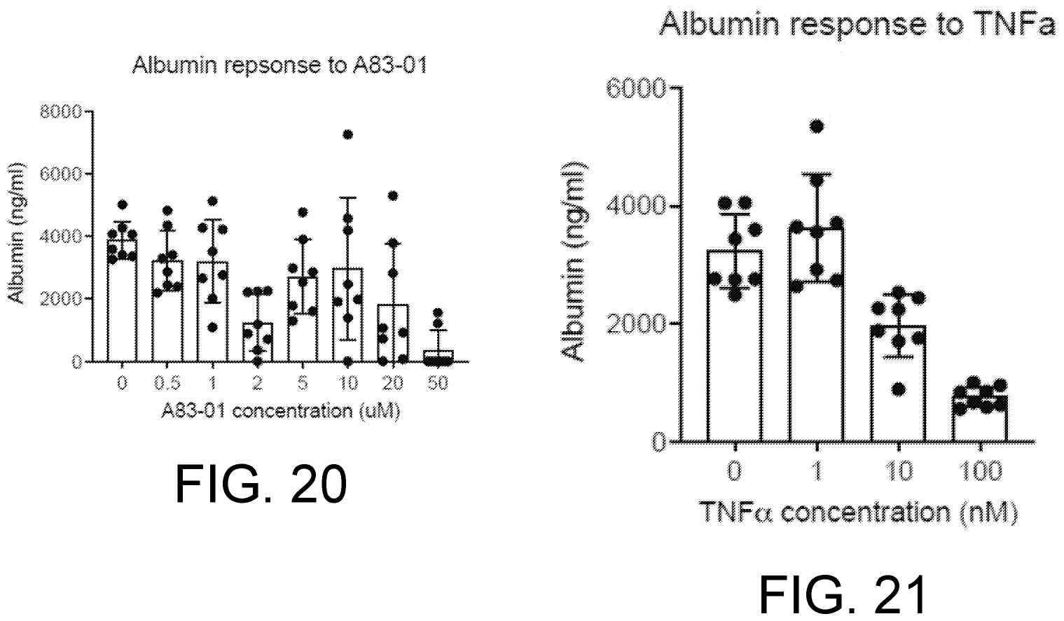

This Example demonstrates benefits of adult hepatocyte medium including TNF-α, HGF, and FGF10. FIG. 19 illustrates albumin secretion measured by human albumin ELISA comparing adult human hepatocyte organoids grown in four different concentrations of TNF-α, ranging from 0 nM to 100 nM. Data is represented as the mean plus or minus the standard deviation of eight wells. FIG. 20 illustrates albumin secretion measured by human albumin ELISA comparing adult human hepatocyte organoids grown in four different medium combinations with or without HGF and FGF10. Data is represented as the mean plus or minus the standard deviation of eight wells.

### Fourth Experimental Example

FIG. 21 illustrates albumin secretion measured by human albumin ELISA comparing adult human hepatocyte organoids grown in media with four different concentrations of TNF-α.

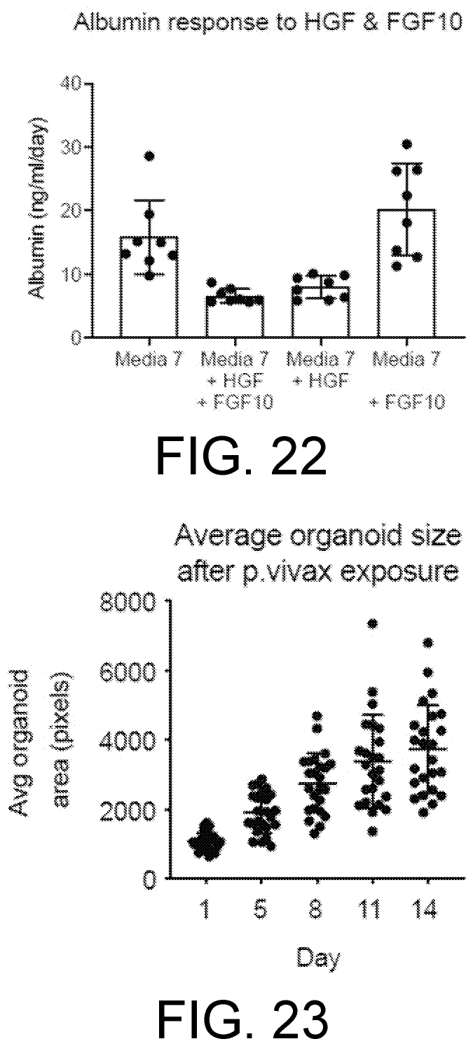

FIG. 22 illustrates albumin secretion measured by human albumin ELISA comparing adult human hepatocyte organoids grown in media with different growth factor compositions, including Hepatocyte Growth Factor (HGF) and Fibroblast Growth Factor 10 (FGF10).

### Fifth Experimental Example

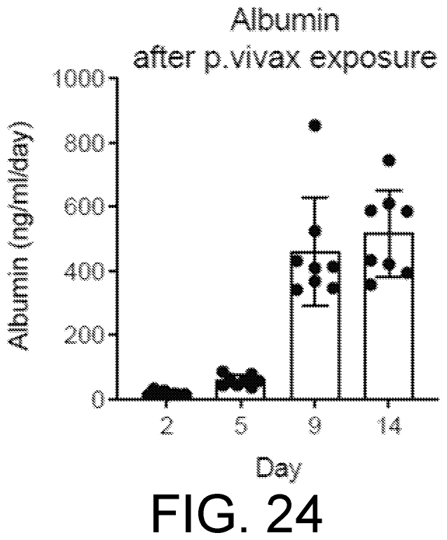

The present Example demonstrates that adult hepatocytes exposed to P. vivax malaria parasites can be used to generate organoids. FIG. 23 illustrates average cross-sectional area of organoids in individual wells from day 1 to day 14 after exposure to P. vivax malaria parasites. Data is represented as mean plus or minus standard deviation of 24 wells. FIG. 24 illustrates albumin secretion measured by human albumin ELISA from day 2 to day 14 after exposure to P. vivax malaria parasites. Data is represented as mean pls or minus standard deviation of 8 wells. Thus, organoids described herein are feasible for infectious disease research applications.

### Sixth Experimental Example

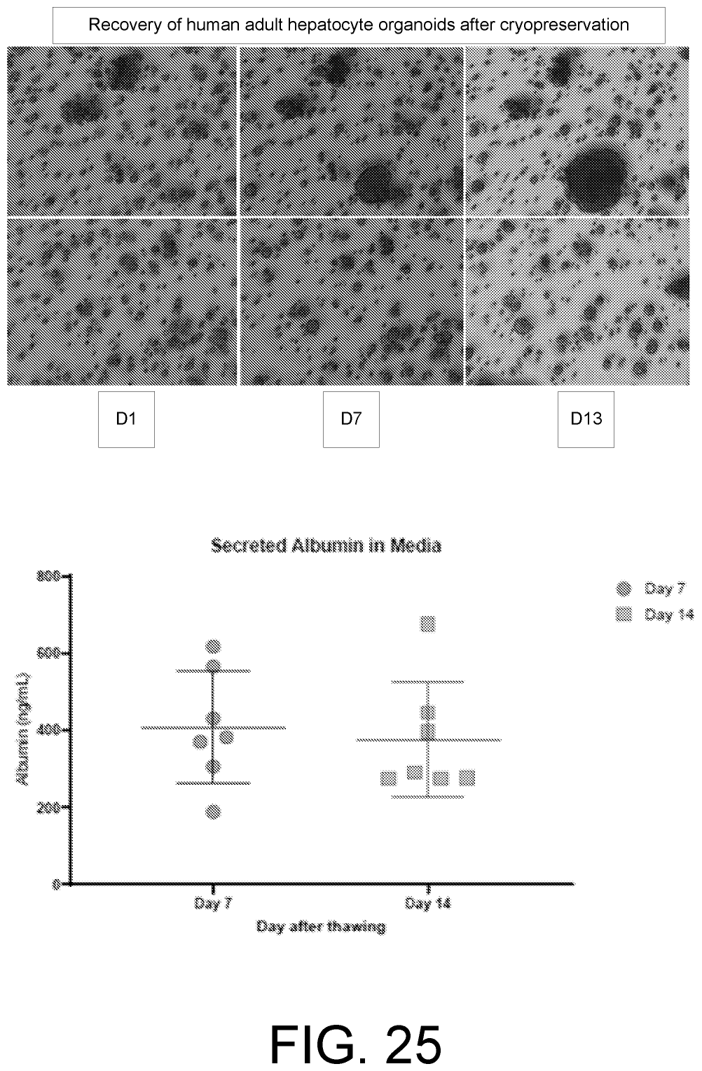

The present Example demonstrates results of an experiment in which human adult hepatocyte organoids were cryopreserved. FIG. 25 illustrates an example of the recovery of human adult hepatocyte organoids after cryopreservation.

### Example Clauses

1. A method including: culturing hepatocytes obtained from an adult donor in an environment including a hepatocyte culture medium, wherein the culturing results in generating an organoid in vitro.
2. The method of clause 1, further including thawing the hepatocytes from a cryopreserved state before the culturing.
3. The method of clause 1 or 2, further including: cryopreserving the hepatocytes from the adult donor; and/or cryopreserving the organoid.
4. The method of any one of clauses 1 to 3, further including obtaining a biopsy of a liver of the adult donor.
5. The method of any one of clauses 1 to 4, wherein the environment further includes a scaffold material.
6. The method of clause 5, wherein the scaffold material includes at least one of a solubilized basement membrane, collagen, fibrin, vitronectin, gelatin methacryloyl (GelMA), polyethylene glycol (PEG), PEG diacrylate (PEGDA), gelatin methacrylate, or a synthetic extracellular matrix.
7. The method of any one of clauses 1 to 6, wherein the hepatocyte culture medium includes 0.1-20 μM of a transforming growth factor-beta (TGF-β) inhibitor.
8. The method of clause 7, wherein the TGF-β inhibitor includes A83-01.
9. The method of any one of clauses 1 to 8, wherein the hepatocyte culture medium includes an epidermal growth factor (EGF), a non-canonical Wnt signaling potentiator, a canonical Wnt potentiator, and a Rho-associated kinase (ROCK) inhibitor.
10. The method of clause 9, wherein the non-canonical Wnt signaling potentiator includes Rspondin1 recombinant protein.
11. The method of clause 9 or 10, wherein the canonical Wnt potentiator includes Wnt3a recombinant protein.
12. The method of any one of clauses 9 to 11, wherein the ROCK inhibitor includes y-27632.
13. The method of any one of clauses 1 to 12, wherein the hepatocyte culture medium includes hepatocyte growth factor (HGF).
14. The method of any one of clauses 1 to 13, wherein the hepatocyte culture medium includes at least one of insulin, norepinephrine, glucagon, dexamethasone, a bile acid, a bile salt, oncostatin M (OSM), TGF-α, insulin-like growth factor (IGF), interleukin (IL)-6, vascular endothelial growth factor (VEGF), IGF-I, IGF-II, prostaglandin E (PGE)-2, IL1-β, a Notch ligand, Ang-2, Activins, a β-catenin pathway signaling potentiator, CHIR99021, fibroblast growth factor (FGF)1, FGF2, FGF7, or FGF10.
15. The method of any one of clauses 1 to 14, wherein the hepatocyte culture medium includes a molecule that specifically stimulates or inhibits at least one of an EGF pathway, an HGF pathway, an FGF pathway, a TNF-α pathway, or a TGF-β pathway.
16. The method of any one of clauses 1 to 15, wherein the hepatocyte culture medium includes 0.1 to 50.0 nM of TNF-α.
17. The method of any one of clauses 1 to 16, wherein the organoid expresses one or more metabolic genes.
18. The method of clause 17, wherein the one or more metabolic genes includes at least one of GAMT, MGST1, HPD, APOA2, APOC1, FABP1, CYP3A4, CYP2E1, or CES1.
19. The method of any one of clauses 1 to 18, wherein the organoid expresses CYP3A4 in response to being exposed to rifampin and/or dexamethasone.
20. The method of any one of clauses 1 to 19, wherein less than a threshold number of cells in the organoid do not express one or more fetal genes.
21. The method of clause 20, wherein the one or more fetal genes include at least one of AFP, CYP3A7, H19, GPC3, DLK1, or SPINK1.
22. The method of any one of clauses 1 to 21, wherein the organoid expresses one or more plasma protein genes.
23. The method of clause 22, wherein the one or more plasma protein genes include at least one of ALB, TTR, or RBP4.
24. The method of any one of clauses 1 to 23, wherein the organoid secretes albumin and/or alpha-1 antitrypsin (A1AT).
25. The method of any one of clauses 1 to 24, wherein the organoid includes cells expressing at least one genetic disease including at least one of nonalcoholic fatty liver disease (NAFLD), hepatocellular carcinoma, A1AT deficiency, tyrosinemia, or hemophilia.
26. The method of any one of clauses 1 to 25, wherein further including culturing fibroblasts and/or endothelial cells with the hepatocytes in the hepatocyte growth medium.
27. The method of any one of clauses 1 to 26, wherein further including culturing one or more of macrophages, natural killer (NK) cells, stellate cells, Kupffer cells, neurons, or adipocytes with the hepatocytes in the hepatocyte culture medium.
28. The method of any one of clauses 1 to 27, further including: implanting the organoid into a subject.
29. The method of any one of clauses 1 to 28, further including: growing an engineered liver or liver tissue using the organoid.
30. The method of any one of clauses 1 to 29, further including: exposing the organoid to a candidate therapeutic; and observing a reaction of the organoid to the candidate therapeutic.
31. The method of clause 30, wherein observing the reaction includes capturing one or more images of the organoid and/or measuring a metabolite of the organoid.
32. A hepatocyte culture medium for culturing adult hepatocytes, the hepatocyte culture medium including: a basal medium; an EGF; a non-canonical Wnt signaling potentiator; a canonical Wnt potentiator; a ROCK inhibitor; and 0.1-20 μM of a TGF-β inhibitor.
33. The hepatocyte culture medium of clause 32, wherein the basal medium includes Dulbecco's Modified Eagle Medium (DMEM) and/or F-12.
34. The hepatocyte culture medium of clause 32 or 33, wherein the basal medium includes at least one of glucose, HEPES, penicillin, streptomycin, nicotinamide, N-acetyl cysteine, gastrin, or a cell culture supplement.
35. The hepatocyte culture medium of any one of clauses 32 to 34, wherein the basal medium includes 1× GlutaMAX, 1-5% B27 by volume, 5-10 mM nicotinamide, 1-1.5 mM N-acetyl cysteine, and 5-15 nM gastrin.
36. The hepatocyte culture medium of any one of clauses 32 to 34, including 30-50% of the basal medium by weight.
37. The hepatocyte culture medium of any one of clauses 32 to 34, wherein the hepatocyte culture medium includes 25-75 ng/mL EGF.
38. The hepatocyte culture medium of any one of clauses 32 to 37, wherein 5-15% of the hepatocyte culture medium by weight includes the non-canonical Wnt signaling potentiator.
39. The hepatocyte culture medium of any one of clauses 32 to 38, wherein the non-canonical Wnt signaling potentiator includes Rspondin1 recombinant protein.
40. The hepatocyte culture medium of any one of clauses 32 to 39, wherein 40-60% of the hepatocyte culture medium by weight includes the canonical Wnt signaling potentiator.
41. The hepatocyte culture medium of any one of clauses 32 to 40, wherein the canonical Wnt signaling potentiator includes Wnt3a recombinant protein.
42. The hepatocyte culture medium of any one of clauses 32 to 41, including 5-10 NM of the ROCK inhibitor, the ROCK inhibitor including y-27632.
43. The hepatocyte culture medium of any one of clauses 32 to 42, wherein the TGF-β inhibitor includes A83-01.
44. The hepatocyte culture medium of any one of clauses 32 to 43, including 0.1-20 uM of the TGF-β inhibitor.
45. The hepatocyte culture medium of any one of clauses 32 to 44, including 5 uM of the TGF-β inhibitor.
46. The hepatocyte culture medium of any one of clauses 32 to 45, further including at least one of insulin, norepinephrine, glucagon, dexamethasone, a bile acid, a bile salt, TGF-α, IGF, IL-6, VEGF, IGF-I, IGF-II, PGE-2, IL1-β, a Notch ligand, Ang-2, Activins, a β-catenin pathway signaling potentiator, CHIR99021, FGF, TNF-α, oncostatin-M, insulin, norepinephrine, glucagon, or dexamethasone.
47. The hepatocyte culture medium of any one of clauses 32 to 46, including 1-100 nM of FGF.
48. The hepatocyte culture medium of any one of clauses 32 to 47, including 10-100 nM TNF-α.
49. The hepatocyte culture medium of any one of clauses 32 to 48, further including a molecule that specifically stimulates or inhibits at least one of an EGF pathway, an HGF pathway, an FGF pathway, a TNF-α pathway, or a TGF-β pathway.
50. A cell culture system including: a culture chamber containing the hepatocyte culture medium of any one of clauses 32 to 49; one or more sensors configured to detect one or more parameters of the culture chamber; one or more actuators; and a processor communicatively coupled to the one or more sensors and the one or more actuators, the processor being configured to control the one or more actuators based on the one or more parameters detected by the one or more sensors.
51. The cell culture system of clause 50, wherein the culture chamber further contains a scaffold material.

The features disclosed in the foregoing description, or the following claims, or the accompanying drawings, expressed in their specific forms or in terms of a means for performing the disclosed function, or a method or process for attaining the disclosed result, as appropriate, may, separately, or in any combination of such features, be used for realizing implementations of the disclosure in diverse forms thereof.

As will be understood by one of ordinary skill in the art, each implementation disclosed herein can comprise, consist essentially of or consist of its particular stated element, step, or component. Thus, the terms “include” or “including” should be interpreted to recite: “comprise, consist of, or consist essentially of.” The transition term “comprise” or “comprises” means has, but is not limited to, and allows for the inclusion of unspecified elements, steps, ingredients, or components, even in major amounts. The transitional phrase “consisting of” excludes any element, step, ingredient or component not specified. The transition phrase “consisting essentially of” limits the scope of the implementation to the specified elements, steps, ingredients or components and to those that do not materially affect the implementation. As used herein, the term “based on” is equivalent to “based at least partly on,” unless otherwise specified.

Unless otherwise indicated, all numbers expressing quantities, properties, conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present disclosure. At the very least, and not as an attempt to limit the application of the doctrine of equivalents to the scope of the claims, each numerical parameter should at least be construed in light of the number of reported significant digits and by applying ordinary rounding techniques. When further clarity is required, the term “about” has the meaning reasonably ascribed to it by a person skilled in the art when used in conjunction with a stated numerical value or range, i.e. denoting somewhat more or somewhat less than the stated value or range, to within a range of ±20% of the stated value; ±19% of the stated value; ±18% of the stated value; ±17% of the stated value; ±16% of the stated value; ±15% of the stated value; ±14% of the stated value; ±13% of the stated value; ±12% of the stated value; ±11% of the stated value; ±10% of the stated value; ±9% of the stated value; ±8% of the stated value; ±7% of the stated value; ±6% of the stated value; ±5% of the stated value; ±4% of the stated value; ±3% of the stated value; ±2% of the stated value; or ±1% of the stated value.

Notwithstanding that the numerical ranges and parameters setting forth the broad scope of the disclosure are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing implementations (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate implementations of the disclosure and does not pose a limitation on the scope of the disclosure. No language in the specification should be construed as indicating any non-claimed element essential to the practice of implementations of the disclosure.

Groupings of alternative elements or implementations disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Variants of the sequences disclosed and referenced herein are also included. Guidance in determining which amino acid residues can be substituted, inserted, or deleted without abolishing biological activity can be found using computer programs well known in the art, such as DNASTAR™ (Madison, Wisconsin) software. Preferably, amino acid changes in the protein variants disclosed herein are conservative amino acid changes, i.e., substitutions of similarly charged or uncharged amino acids. A conservative amino acid change involves substitution of one of a family of amino acids which are related in their side chains.

In a peptide or protein, suitable conservative substitutions of amino acids are known to those of skill in this art and generally can be made without altering a biological activity of a resulting molecule. Those of skill in this art recognize that, in general, single amino acid substitutions in non-essential regions of a polypeptide do not substantially alter biological activity (see, e.g., Watson et al. Molecular Biology of the Gene, 4th Edition, 1987, The Benjamin/Cummings Pub. Co., p. 224). Naturally occurring amino acids are generally divided into conservative substitution families as follows: Group 1: Alanine (Ala), Glycine (Gly), Serine (Ser), and Threonine (Thr); Group 2: (acidic): Aspartic acid (Asp), and Glutamic acid (Glu); Group 3: (acidic; also classified as polar, negatively charged residues and their amides): Asparagine (Asn), Glutamine (Gln), Asp, and Glu; Group 4: Gln and Asn; Group 5: (basic; also classified as polar, positively charged residues): Arginine (Arg), Lysine (Lys), and Histidine (His); Group 6 (large aliphatic, nonpolar residues): Isoleucine (Ile), Leucine (Leu), Methionine (Met), Valine (Val) and Cysteine (Cys); Group 7 (uncharged polar): Tyrosine (Tyr), Gly, Asn, Gln, Cys, Ser, and Thr; Group 8 (large aromatic residues): Phenylalanine (Phe), Tryptophan (Trp), and Tyr; Group 9 (non-polar): Proline (Pro), Ala, Val, Leu, lie, Phe, Met, and Trp; Group 11 (aliphatic): Gly, Ala, Val, Leu, and lie; Group 10 (small aliphatic, nonpolar or slightly polar residues): Ala, Ser, Thr, Pro, and Gly; and Group 12 (sulfur-containing): Met and Cys. Additional information can be found in Creighton (1984) Proteins, W.H. Freeman and Company.

In making such changes, the hydropathic index of amino acids may be considered. The importance of the hydropathic amino acid index in conferring interactive biologic function on a protein is generally understood in the art (Kyte and Doolittle, 1982, J. Mol. Biol. 157(1), 105-32). Each amino acid has been assigned a hydropathic index on the basis of its hydrophobicity and charge characteristics (Kyte and Doolittle, 1982). These values are: Ile (+4.5); Val (+4.2); Leu (+3.8); Phe (+2.8); Cys (+2.5); Met (+1.9); Ala (+1.8); Gly (−0.4); Thr (−0.7); Ser (−0.8); Trp (−0.9); Tyr (−1.3); Pro (−1.6); His (−3.2); Glutamate (−3.5); Gln (−3.5); aspartate (−3.5); Asn (−3.5); Lys (−3.9); and Arg (−4.5).

It is known in the art that certain amino acids may be substituted by other amino acids having a similar hydropathic index or score and still result in a protein with similar biological activity, i.e., still obtain a biological functionally equivalent protein. In making such changes, the substitution of amino acids whose hydropathic indices are within ±2 is preferred, those within ±1 are particularly preferred, and those within ±0.5 are even more particularly preferred. It is also understood in the art that the substitution of like amino acids can be made effectively on the basis of hydrophilicity.

As detailed in U.S. Pat. No. 4,554,101, the following hydrophilicity values have been assigned to amino acid residues: Arg (+3.0); Lys (+3.0); aspartate (+3.0±1); glutamate (+3.0±1); Ser (+0.3); Asn (+0.2); Gln (+0.2); Gly (0); Thr (−0.4); Pro (−0.5±1); Ala (−0.5); His (−0.5); Cys (−1.0); Met (−1.3); Val (−1.5); Leu (−1.8); Ile (−1.8); Tyr (−2.3); Phe (−2.5); Trp (−3.4). It is understood that an amino acid can be substituted for another having a similar hydrophilicity value and still obtain a biologically equivalent, and in particular, an immunologically equivalent protein. In such changes, the substitution of amino acids whose hydrophilicity values are within ±2 is preferred, those within ±1 are particularly preferred, and those within ±0.5 are even more particularly preferred.

As outlined above, amino acid substitutions may be based on the relative similarity of the amino acid side-chain substituents, for example, their hydrophobicity, hydrophilicity, charge, size, and the like. As indicated elsewhere, variants of gene sequences can include codon optimized variants, sequence polymorphisms, splice variants, and/or mutations that do not affect the function of an encoded product to a statistically-significant degree.

Variants of the nucleic acid sequences disclosed herein include sequences with at least 70% sequence identity, 80% sequence identity, 85% sequence, 90% sequence identity, 95% sequence identity, 96% sequence identity, 97% sequence identity, 98% sequence identity, or 99% sequence identity to the protein, nucleic acid, or gene sequences disclosed herein.

“% sequence identity” refers to a relationship between two or more sequences, as determined by comparing the sequences. In the art, “identity” also means the degree of sequence relatedness between protein, nucleic acid, or gene sequences as determined by the match between strings of such sequences. “Identity” (often referred to as “similarity”) can be readily calculated by known methods, including those described in: Computational Molecular Biology (Lesk, A. M., ed.) Oxford University Press, NY (1988); Biocomputing: Informatics and Genome Projects (Smith, D. W., ed.) Academic Press, NY (1994); Computer Analysis of Sequence Data, Part I (Griffin, A. M., and Griffin, H. G., eds.) Humana Press, NJ (1994); Sequence Analysis in Molecular Biology (Von Heijne, G., ed.) Academic Press (1987); and Sequence Analysis Primer (Gribskov, M. and Devereux, J., eds.) Oxford University Press, NY (1992). Preferred methods to determine identity are designed to give the best match between the sequences tested. Methods to determine identity and similarity are codified in publicly available computer programs. Sequence alignments and percent identity calculations may be performed using the Megalign program of the LASERGENE bioinformatics computing suite (DNASTAR, Inc., Madison, Wisconsin). Multiple alignment of the sequences can also be performed using the Clustal method of alignment (Higgins and Sharp CABIOS, 5, 151-153 (1989) with default parameters (GAP PENALTY=10, GAP LENGTH PENALTY=10). Relevant programs also include the GCG suite of programs (Wisconsin Package Version 9.0, Genetics Computer Group (GCG), Madison, Wisconsin); BLASTP, BLASTN, BLASTX (Altschul, et al., J. Mol. Biol. 215:403-410 (1990); DNASTAR (DNASTAR, Inc., Madison, Wisconsin); and the FASTA program incorporating the Smith-Waterman algorithm (Pearson, Comput. Methods Genome Res., [Proc. Int. Symp.] (1994), Meeting Date 1992, 111-20. Editor(s): Suhai, Sandor. Publisher: Plenum, New York, N.Y. Within the context of this disclosure it will be understood that where sequence analysis software is used for analysis, the results of the analysis are based on the “default values” of the program referenced. As used herein “default values” will mean any set of values or parameters, which originally load with the software when first initialized.

Variants also include nucleic acid molecules that hybridizes under stringent hybridization conditions to a sequence disclosed herein and provide the same function as the reference sequence. Exemplary stringent hybridization conditions include an overnight incubation at 42° C. in a solution including 50% formamide, 5×SSC (750 mM NaCl, 75 mM trisodium citrate), 50 mM sodium phosphate (pH 7.6), 5×Denhardt's solution, 10% dextran sulfate, and 20 μg/ml denatured, sheared salmon sperm DNA, followed by washing the filters in 0.1×SSC at 50° C. Changes in the stringency of hybridization and signal detection are primarily accomplished through the manipulation of formamide concentration (lower percentages of formamide result in lowered stringency); salt conditions, or temperature. For example, moderately high stringency conditions include an overnight incubation at 37° C. in a solution including 6×SSPE (20×SSPE=3M NaCl; 0.2M NaH2PO4; 0.02M EDTA, pH 7.4), 0.5% SDS, 30% formamide, 100 μg/ml salmon sperm blocking DNA; followed by washes at 50° C. with 1×SSPE, 0.1% SDS. In addition, to achieve even lower stringency, washes performed following stringent hybridization can be done at higher salt concentrations (e.g. 5×SSC). Variations in the above conditions may be accomplished through the inclusion and/or substitution of alternate blocking reagents used to suppress background in hybridization experiments. Typical blocking reagents include Denhardt's reagent, BLOTTO, heparin, denatured salmon sperm DNA, and commercially available proprietary formulations. The inclusion of specific blocking reagents may require modification of the hybridization conditions described above, due to problems with compatibility.

Unless otherwise indicated, the practice of the present disclosure can employ conventional techniques of immunology, molecular biology, microbiology, cell biology and recombinant DNA. These methods are described in the following publications. See, e.g., Sambrook, et al. Molecular Cloning: A Laboratory Manual, 2nd Edition (1989); F. M. Ausubel, et al. eds., Current Protocols in Molecular Biology, (1987); the series Methods IN Enzymology (Academic Press, Inc.); M. MacPherson, et al., PCR: A Practical Approach, IRL Press at Oxford University Press (1991); MacPherson et al., eds. PCR 2: Practical Approach, (1995); Harlow and Lane, eds. Antibodies, A Laboratory Manual, (1988); and R. I. Freshney, ed. Animal Cell Culture (1987).

Certain implementations are described herein, including the best mode known to the inventors for carrying out implementations of the disclosure. Of course, variations on these described implementations will become apparent to those of ordinary skill in the art upon reading the foregoing description. The inventor expects skilled artisans to employ such variations as appropriate, and the inventors intend for implementations to be practiced otherwise than specifically described herein. Accordingly, the scope of this disclosure includes all modifications and equivalents of the subject matter recited in the claims appended hereto as permitted by applicable law. Moreover, any combination of the above-described elements in all possible variations thereof is encompassed by implementations of the disclosure unless otherwise indicated herein or otherwise clearly contradicted by context.

Furthermore, numerous references have been made to patents, printed publications, journal articles and other written text throughout this specification (referenced materials herein). Each of the referenced materials are individually incorporated herein by reference in their entirety for their referenced teaching.

What is claimed is:

1. A method comprising:
culturing hepatocytes obtained from an adult donor in an environment comprising a hepatocyte culture medium, wherein the culturing results in generating an organoid in vitro.

2. The method of claim 1, further comprising thawing the hepatocytes from a cryopreserved state before the culturing.

3. The method of claim 1, further comprising:
cryopreserving the hepatocytes from the adult donor; and/or
cryopreserving the organoid.

4. The method of claim 1, further comprising obtaining a biopsy of a liver of the adult donor.

5. The method of claim 1, wherein the environment further comprises a scaffold material.

6. The method of claim 5, wherein the scaffold material comprises at least one of a solubilized basement membrane, collagen, fibrin, vitronectin, gelatin methacryloyl (GelMA), polyethylene glycol (PEG), PEG diacrylate (PEGDA), gelatin methacrylate, or a synthetic extracellular matrix.

7. The method of claim 1, wherein the hepatocyte culture medium comprises 0.1-20 μM of a transforming growth factor-beta (TGF-β) inhibitor.

8. The method of claim 7, wherein the TGF-β inhibitor comprises A83-01.

9. The method of claim 1, wherein the hepatocyte culture medium comprises an epidermal growth factor (EGF), a non-canonical Wnt signaling potentiator, a canonical Wnt potentiator, and a Rho-associated kinase (ROCK) inhibitor.

10. The method of claim 9, wherein the non-canonical Wnt signaling potentiator comprises Rspondin1 recombinant protein.

11. The method of claim 9, wherein the canonical Wnt potentiator comprises Wnt3a recombinant protein.

12. The method of claim 9, wherein the ROCK inhibitor comprises y-27632.

13. The method of claim 1, wherein the hepatocyte culture medium comprises hepatocyte growth factor (HGF).

14. The method of claim 1, wherein the hepatocyte culture medium comprises at least one of insulin, norepinephrine, glucagon, dexamethasone, a bile acid, a bile salt, oncostatin M (OSM), TGF-α, insulin-like growth factor (IGF), interleukin (IL)-6, vascular endothelial growth factor (VEGF), IGF-I, IGF-II, prostaglandin E (PGE)-2, IL1-β, a Notch ligand, Ang-2, Activins, a β-catenin pathway signaling potentiator, CHIR99021, fibroblast growth factor (FGF)1, FGF2, FGF7, or FGF10.

15. The method of claim 1, wherein the hepatocyte culture medium comprises a molecule that specifically stimulates or inhibits at least one of an EGF pathway, an HGF pathway, an FGF pathway, a TNF-α pathway, or a TGF-β pathway.

16. The method of claim 1, wherein the hepatocyte culture medium comprises 0.1 to 50.0 nM of TNF-α.

17. The method of claim 1, wherein the organoid expresses one or more metabolic genes.

18. The method of claim 17, wherein the one or more metabolic genes comprises at least one of GAMT, MGST1, HPD, APOA2, APOC1, FABP1, CYP3A4, CYP2E1, or CES1.

19. The method of claim 1, wherein the organoid expresses CYP3A4 in response to being exposed to rifampin and/or dexamethasone.

20. The method of claim 1, wherein less than a threshold number of cells in the organoid do not express one or more fetal genes.

21. The method of claim 20, wherein the one or more fetal genes comprise at least one of AFP, CYP3A7, H19, GPC3, DLK1, or SPINK1.

22. The method of claim 1, wherein the organoid expresses one or more plasma protein genes.

23. The method of claim 22, wherein the one or more plasma protein genes comprise at least one of ALB, TTR, or RBP4.

24. The method of claim 1, wherein the organoid secretes albumin and/or alpha-1 antitrypsin (A1AT).

25. The method of claim 1, wherein the organoid comprises cells expressing at least one genetic disease comprising at least one of nonalcoholic fatty liver disease (NAFLD), hepatocellular carcinoma, A1AT deficiency, tyrosinemia, or hemophilia.

26. The method of claim 1, wherein further comprising culturing fibroblasts and/or endothelial cells with the hepatocytes in the hepatocyte growth medium.

27. The method of claim 1, wherein further comprising culturing one or more of macrophages, natural killer (NK) cells, stellate cells, Kupffer cells, neurons, or adipocytes with the hepatocytes in the hepatocyte culture medium.

28. The method of claim 1, further comprising:
implanting the organoid into a subject.

29. The method of claim 1, further comprising:
growing an engineered liver or liver tissue using the organoid.

30. The method of claim 1, further comprising:
exposing the organoid to a candidate therapeutic; and
observing a reaction of the organoid to the candidate therapeutic.

31. The method of claim 30, wherein observing the reaction comprises capturing one or more images of the organoid and/or measuring a metabolite of the organoid.

32. A hepatocyte culture medium for culturing adult hepatocytes, the hepatocyte culture medium comprising:
a basal medium;
an EGF;
a non-canonical Wnt signaling potentiator;
a canonical Wnt potentiator;
a ROCK inhibitor; and
0.1-20 μM of a TGF-β inhibitor.

33. The hepatocyte culture medium of claim 32, wherein the basal medium comprises Dulbecco's Modified Eagle Medium (DMEM) and/or F-12.

34. The hepatocyte culture medium of claim 32, wherein the basal medium comprises at least one of glucose, HEPES, penicillin, streptomycin, nicotinamide, N-acetyl cysteine, gastrin, or a cell culture supplement.

35. The hepatocyte culture medium of claim 32, wherein the basal medium includes 1×GlutaMAX, 1-5% B27 by volume, 5-10 mM nicotinamide, 1-1.5 mM N-acetyl cysteine, and 5-15 nM gastrin.

36. The hepatocyte culture medium of claim 32, comprising 30-50% of the basal medium by weight.

37. The hepatocyte culture medium of claim 32, wherein the hepatocyte culture medium comprises 25-75 ng/mL EGF.

38. The hepatocyte culture medium of claim 32, wherein 5-15% of the hepatocyte culture medium by weight comprises the non-canonical Wnt signaling potentiator.

39. The hepatocyte culture medium of claim 32, wherein the non-canonical Wnt signaling potentiator comprises Rspondin1 recombinant protein.

40. The hepatocyte culture medium of claim 32, wherein 40-60% of the hepatocyte culture medium by weight comprises the canonical Wnt signaling potentiator.

41. The hepatocyte culture medium of claim 32, wherein the canonical Wnt signaling potentiator comprises Wnt3a recombinant protein.

42. The hepatocyte culture medium of claim 32, comprising 5-10 μM of the ROCK inhibitor, the ROCK inhibitor comprising y-27632.

43. The hepatocyte culture medium of claim 32, wherein the TGF-β inhibitor comprises A83-01.

44. The hepatocyte culture medium of claim 32, comprising 0.1-20 uM of the TGF-β inhibitor.

45. The hepatocyte culture medium of claim 32, comprising 5 uM of the TGF-β inhibitor.

46. The hepatocyte culture medium of claim 32, further comprising at least one of insulin, norepinephrine, glucagon, dexamethasone, a bile acid, a bile salt, TGF-α, IGF, IL-6, VEGF, IGF-I, IGF-II, PGE-2, IL1-β, a Notch ligand, Ang-2, Activins, a β-catenin pathway signaling potentiator, CHIR99021, FGF, TNF-α, oncostatin-M, insulin, norepinephrine, glucagon, or dexamethasone.

47. The hepatocyte culture medium of claim 32, comprising 1-100 nM of FGF.

48. The hepatocyte culture medium of claim 32, comprising 10-100 nM TNF-α.

49. The hepatocyte culture medium of claim 32, further comprising a molecule that specifically stimulates or inhibits at least one of an EGF pathway, an HGF pathway, an FGF pathway, a TNF-α pathway, or a TGF-β pathway.

50. A cell culture system comprising:
a culture chamber containing the hepatocyte culture medium of claim 32;
one or more sensors configured to detect one or more parameters of the culture chamber;
one or more actuators; and
a processor communicatively coupled to the one or more sensors and the one or more actuators, the processor being configured to control the one or more actuators based on the one or more parameters detected by the one or more sensors.

51. The cell culture system of claim 50, wherein the culture chamber further contains a scaffold material.


SEQUENCE LISTING (16 sequences)

SEQ ID NO: 1 - DNA, 23 bp, synthetic construct
  source 1..23: mol_type = other DNA
gatcaacgattacgtggagaagg

SEQ ID NO: 2 - DNA, 22 bp, synthetic construct
  source 1..22: mol_type = other DNA
cctaaacgcttcatcataggca

SEQ ID NO: 3 - DNA, 22 bp, synthetic construct
  source 1..22: mol_type = other DNA
agtgaggacaaactattggcct

SEQ ID NO: 4 - DNA, 21 bp, synthetic construct
  source 1..21: mol_type = other DNA
acaccagggtttactggagtc

SEQ ID NO: 5 - DNA, 22 bp, synthetic construct
  source 1..22: mol_type = other DNA
gagaccagaggttgatgtgatg

SEQ ID NO: 6 - DNA, 22 bp, synthetic construct
  source 1..22: mol_type = other DNA
agttccggggcataaaagtaag

SEQ ID NO: 7 - DNA, 20 bp, synthetic construct
  source 1..20: mol_type = other DNA
gccaagtccagaaccatagg

SEQ ID NO: 8 - DNA, 20 bp, synthetic construct
  source 1..20: mol_type = other DNA
agcagaccagcctttctcaa

SEQ ID NO: 9 - DNA, 20 bp, synthetic construct
  source 1..20: mol_type = other DNA
tccgaaccaagtttgagacg

SEQ ID NO: 10 - DNA, 21 bp, synthetic construct
  source 1..21: mol_type = other DNA
ccctcagcgtactgatttcct

SEQ ID NO: 11 - DNA, 20 bp, synthetic construct
  source 1..20: mol_type = other DNA
cagcctggtgctcctctatc

SEQ ID NO: 12 - DNA, 20 bp, synthetic construct
  source 1..20: mol_type = other DNA
accatcataaaagccccaca

SEQ ID NO: 13 - DNA, 20 bp, synthetic construct
  source 1..20: mol_type = other DNA
accttgtgaccaagcctgag

SEQ ID NO: 14 - DNA, 20 bp, synthetic construct
  source 1..20: mol_type = other DNA
aaggaggagtgtcggaaggt

SEQ ID NO: 15 - DNA, 20 bp, synthetic construct
  source 1..20: mol_type = other DNA
tgattgcatcagggccattg

SEQ ID NO: 16 - DNA, 22 bp, synthetic construct
  source 1..22: mol_type = other DNA
gccaggtaagcatcatacacca